(19) United States
(12) Patent Application Publication
(10) Pub. No.: US 2025/0243549 A1
(43) Pub. Date: Jul. 31, 2025

(54) Reproducible Double Unique Dual Indexing Library Construction Method for Next Generation Sequencing of microRNA Isoform and Use Thereof

(71) Applicant: Jiansheng Medical Technology Co., Ltd., Beijing (CN)

(72) Inventors: Jiawang WANG, Beijing (CN); Bin ZHANG, Beijing (CN)

(21) Appl. No.: 18/425,986

(22) Filed: Jan. 29, 2024

(51) Int. Cl. C12Q 1/6886; C12Q 1/6806; C12Q 1/6851; C12Q 1/6874; G16B 30/00; G16B 40/20

(52) U.S. Cl. CPC C12Q 1/6886; C12Q 1/6851; C12Q 1/6874; G16B 30/00; G16B 40/20; C12Q 1/6806; C12Q 2600/16; C12Q 2600/178

(57) ABSTRACT

The present disclosure provides a reproducible double unique dual indexing library construction method for next generation sequencing of a microRNA isoform (isomiR) and a use thereof, and belongs to the technical field of gene sequencing. The present disclosure discloses a primer set for amplification of an isomiR, including a universal sequence and a 5′-terminus amplification primer linked sequentially to a partial sequence of a 5′ terminus of a miRNA. The primer set can allow the amplification of different isoforms of a microRNA (miRNA), with characteristics such as high sensitivity, high relative sequencing depth, and high specificity. The present disclosure also discloses a double unique dual indexing technology for multiplex next-generation sequencing (NGS) to solve the problems of NGS of high-throughput samples, and the double unique dual indexing technology has characteristics such as excellent repeatability, high detection accuracy, and low detection cost and can allow the artificial intelligent diagnosis of a tumor based on NGS.

### TECHNICAL FIELD

The present disclosure belongs to the technical field of gene sequencing, and specifically relates to a reproducible double unique dual indexing library construction method for next generation sequencing of a microRNA isoform (isomiR) and a use thereof.

### REFERENCE TO SEQUENCE LISTING

A computer readable XML file entitled “GWP20240100524_seqlist”, that was created on Mar. 28, 2024, with a file size of about 1,812,085 bytes, contains the sequence listing for this application, has been filed with this application, and is hereby incorporated by reference in its entirety.

### BACKGROUND

microRNAs (miRNAs) are ideal biomarkers for cancers, miRNAs are a class of small non-coding RNAs each with a length of 18 to 25 nucleotides, miRNAs directly and indirectly regulate the expression of most genes, and participate in a series of life activities, including cell proliferation, apoptosis, organogenesis, hematopoiesis, and development, miRNAs are closely related to the occurrence and progression of tumors. Increasing studies have shown that miRNAs play an important regulatory role in the occurrence and progression of tumors. Malignant tumors are results of an interaction between genetic factors and environmental factors, where environmental factors play a greater role than genetic factors. The genetic diagnosis of cancers has great limitations, and can only discover susceptibility genes, which cannot be used as biomarkers for the diagnosis of malignant tumors. In addition, environmental factors are not monitorable and can only serve as risk factors for malignant tumors, miRNAs are a large class of regulatory factors between changing environments and unchanging genetic materials, and are major bridges connecting environmental factors and genetic factors. Therefore, miRNAs may be desirable biomarkers for malignant tumors, miRNAs are very stable in blood, and plasma miRNAs are relatively stable under harsh conditions such as freezing and thawing, high-temperature (up to 37° C.) storage, acidic conditions, and ribonuclease digestion. Compared with protein markers and mRNA expression profiles, miRNA expression abnormalities appear earlier, can be used to more accurately distinguish tumor types, are more beneficial for early diagnosis, and are more suitable as markers for tumor diagnosis. Due to these characteristics, miRNAs are very attractive as non-invasive biomarkers, and are suitable as biomarkers for diseases. Recent evidences have shown that circulating miRNAs in blood can be used as biomarkers for the etiology, diagnosis, progression, recurrence, and treatment outcomes of tumors.

Based on the principle of base pairing, miRNAs can bind to messenger RNAs (mRNAs) to specifically inhibit the translation of the mRNAs. About more than 8,000 miRNAs have been discovered in humans. Each miRNA can regulate the expression of hundreds or even thousands of genes. Moreover, miRNAs, like hormones, can be secreted by cells into a blood circulation flow and delivered to other adjacent or distant cells to play a role. Therefore, miRNAs directly or indirectly regulate almost all genes and regulate various functions of cells. Indeed, miRNAs can reverse cancer cells into normal cells and turn differentiated cells into stem cells, and the knockout of miRNAs in mice is embryonically lethal.

Due to an important role of miRNAs in gene regulation, the abnormal expression of miRNAs is closely related to various diseases such as cancers. It has been found that the dysregulation of miRNAs is associated with more than 400 diseases. When a body is endangered by pathogenic microorganisms or cancer cells, an immune response requires the rapid and highly-coordinated systemic regulation of many genes to establish an effective defense to identify and eliminate pathogenic factors. The miRNA-mediated gene regulation is faster than other epigenetic mechanisms (such as methylation) that require transcription. Only a miRNA regulatory network can meet the need of such rapid gene regulation.

Compared with other molecular assays, miRNA assays undoubtedly have tremendous advantages, miRNAs are very stable in blood, and plasma miRNAs are stable under harsh conditions such as freezing and thawing, high-temperature storage, acidic conditions, and ribonuclease digestion. As a result, miRNAs are very attractive as biomarkers, and are very suitable as biomarkers for diseases. The clinical applied research of miRNAs has become one of the hot spots. However, the research on miRNAs as biomarkers for gastric cancer inside and outside China is still at a laboratory research stage, and miRNAs have not been successfully used in the clinical diagnosis of gastric cancer.

Through detailed and accurate analysis of miRNA sequences by a high-throughput sequencing technology, isomiRs are discovered (Gómez-Martín C, Aparicio-Puerta E, van Eijndhoven M A, et al.). Accordingly, the early belief that each miRNA gene produces only one mature miRNA sequence is overturned. A miRNA is not a single sequence, but consists of a series of isomiRs with different lengths/sequences and expressions. These isomiRs are different from cach other merely in one or a few bases. These isomiRs are diverse in expressions and sequences, and even introduce a variety of 5′ termini and seed regions. Specific miRNA loci can have abnormal expression patterns in diseased tissues. Some isomiRs have been proved to have important biological functions. Mechanisms for producing isomiRs mainly include: inaccurate or selective cleavage of Darsha and Dicer enzymes during miRNA processing and maturation; addition of a nucleotide at a 3′ terminus; and RNA editing and single nucleotide polymorphism (SNP). Major manifestations include: 5′-terminus trimming, 3′-terminus trimming, 3′-terminus nucleotide addition, and base substitution. The 5′-terminus trimming and base substitution can occur within a seed region, resulting in seed shifting. The expression of different isoforms of a same miRNA varies greatly and is tissue-specific. In particular, the expression specificity of an isoform in a pathological tissue can be used as a biomarker for diagnosis of a disease. IsomiR is a functional and independent molecule that can regulate the expression of a gene like a corresponding precursor of the isomiR, and the expression of isomiR is accurately regulated in different tissues under different pathological conditions. Each miRNA seems to have a large number of isoforms. Therefore, the research and application of miRNAs should go deep into an isomiR level to obtain accurate results. Comparatively, there are many isomiRs at a 3′ terminus.

Because miRNAs each include only about 20 bases and are at a low level in blood, it is difficult to detect miRNAs, and there is a lack of techniques to accurately detect miRNAs. Quantitative polymerase chain reaction (qPCR), microarrays, and small RNA sequencing (RNA-seq) are commonly used in the research on expression of miRNAs in tissues. However, these techniques all have defects to varying degrees. The microarray technique mainly has problems such as low sensitivity and relatively-long turnaround time, and the qPCR technique is not easy to detect a large number of miRNAs. In numerous studies, study results of circulating miRNAs have extremely-low reproducibility. Detection results of different laboratories are not comparable to each other, and may even be opposite to each other. Summarized results of 11 studies show that 31 miRNAs associated with heart failure are identified in one study, and only five of the miRNAs can be reproduced in another study, but none of the miRNAs can be replicated in more than two studies, which fully indicates that the existing qPCR technique for detecting miRNAs has serious shortcomings. These shortcomings greatly limit the application of the qPCR technique in clinical quantitative detection of extracellular miRNAs. For example, miRNAs have not been successfully used in the cancer diagnosis.

In addition, next-generation sequencing (NGS) (small RNA-seq), as a rising star, has received extremely-extensive attention due to its advantages such as high versatility and accuracy to a single base, and can be used for the detection of gene expression. The detection of gene expression should in fact be the largest application market for NGS. Unfortunately, the first half of steps of NGS to detect the gene expression are the same as the first half of steps of qPCR to detect the gene expression, and thus the problems of NGS to detect the gene expression are also faced by qPCR. The qPCR technique mentioned above has serious shortcomings, and thus the qPCR technique must be subversively innovated to allow the successful application of the qPCR technique in clinical practice, which is also applicable to the NGS to detect the gene expression.

While the NGS small RNA-seq works excellently for the discovery of new miRNAs, the NGS small RNA-seq is not suitable for applications requiring high-throughput samples or fast turnarounds. In order to improve the efficiency, a capacity of a sequencing chip should be as high as possible. NGS can produce a large amount of data, for example, 6,000 Gb (6 Tb) of data can be acquired at a time when Illumina sequencing is conducted with an S4 flow cell. In addition, a quantity of sequencing data of a sample is often relatively small. As a result, a plurality of samples often needs to be pooled for sequencing. To allow this objective, each sample needs to be labeled specifically. Although the current unique dual indexing technology can theoretically label thousands of samples, the labeling of high-throughput samples is not possible due to various difficulties in practice. About 100 samples are adopted at most for the miRNA sequencing in the literature. In view of the huge data quantity that can be produced by the technique, such a small sample quantity is far from sufficient. Ideally, in clinical applications, tens of thousands of patient samples should be treated in a single run.

### SUMMARY

In view of this, a first objective of the present disclosure is to provide a primer set for amplification of an isomiR, including a universal sequence and a primer linked to a partial sequence of a 5′ terminus of a miRNA. The primer set allows the amplification of different isoforms of a same miRNA.

A second objective of the present disclosure is to provide a double unique dual indexing amplification primer set for construction of a high-throughput sample library for NGS, including an inner dual index and an outer dual index. When the primer set including a combination of an outer unique dual index (OUDI) and an inner unique dual index (IUDI) is used to amplify samples and then amplification products are pooled, detection requirements of high-throughput samples can be met.

The present disclosure provides a primer set for amplification of an isomiR, including a universal sequence and a 5′-terminus amplification primer linked sequentially to a partial sequence of a 5′ terminus of a miRNA.

Preferably, the miRNA includes at least one selected from the group consisting of the following: hsa-miR-21-5p, hsa-miR-223-3p, hsa-miR-223-5p, hsa-miR-186-5p, hsa-miR-18a-5p, hsa-miR-146b-5p, hsa-miR-624-5p, hsa-miR-106b-5p, hsa-miR-340-5p, hsa-miR-20a-5p, hsa-miR-451a, hsa-miR-7976, hsa-miR-2355-3p, hsa-miR-301a-3p, hsa-miR-144-5p, hsa-miR-151a-3p, hsa-miR-3200-5p, hsa-miR-1537-3p, hsa-miR-500a-5p, hsa-miR-127-3p, hsa-miR-570-3p, hsa-miR-130b-5p, hsa-miR-503-5p, hsa-miR-551a, hsa-miR-409-3p, hsa-miR-330-3p, hsa-miR-889-3p, hsa-miR-625-5p, hsa-miR-542-3p, hsa-miR-582-3p, hsa-miR-381-3p, hsa-miR-495-3p, hsa-miR-103a-1-5p, hsa-miR-450b-5p, hsa-miR-429, hsa-miR-576-5p, hsa-miR-148b-3p, hsa-miR-320c, hsa-miR-4286, hsa-miR-126-3p, hsa-miR-152-3p, hsa-miR-144-3p, hsa-miR-195-5p, hsa-let-7a-5p, hsa-miR-378f, hsa-miR-126-5p, hsa-miR-26a-5p, hsa-miR-29a-3p, hsa-miR-181a-5p, hsa-miR-32-5p, hsa-miR-142-3p, hsa-miR-29c-3p, hsa-miR-424-5p, hsa-miR-143-3p, hsa-miR-30c-5p, hsa-miR-192-5p, hsa-miR-146a-5p, hsa-miR-101-3p, hsa-miR-19b-3p, hsa-miR-33b-5p, hsa-miR-378a-3p, hsa-miR-22-3p, hsa-miR-107, hsa-miR-497-5p, hsa-miR-15a-3p, hsa-miR-188-5p, hsa-let-7d-3p, hsa-miR-132-3p, hsa-miR-151a-5p, hsa-miR-194-5p, hsa-miR-99a-5p, hsa-miR-125b-5p, hsa-miR-25-3p, hsa-miR-103a-3p, hsa-miR-1285-3p, hsa-miR-7977, hsa-miR-30b-5p, hsa-miR-363-3p, hsa-miR-93-5p, hsa-miR-375-3p, hsa-miR-99b-5p, hsa-miR-193b-3p, hsa-miR-324-3p, hsa-miR-193a-3p, hsa-miR-342-3p, hsa-miR-484, hsa-miR-532-3p, hsa-miR-210-3p, hsa-miR-2110, hsa-miR-296-5p, hsa-miR-1307-5p, hsa-miR-19a-3p, hsa-miR-139-5p, hsa-miR-3665, hsa-miR-RG-84, hsa-miR-4454, and hsa-let-7b-5p; and
nucleotide sequences of corresponding 5′-terminus amplification primers are shown in SEQ ID NO: 1 to SEQ ID NO: 97, respectively.

Preferably, the primer set for amplification of an isomiR further includes a second polymerase chain reaction (PCR) preamplification primer pair and/or a third PCR preamplification primer pair;
the second PCR preamplification primer pair includes a transition primer and a reverse primer for amplifying the isomiR;
a nucleotide sequence of the transition primer is shown in SEQ ID NO: 99;
a nucleotide sequence of the reverse primer for amplifying the isomiR is shown in SEQ ID NO: 100;
the third PCR preamplification primer pair includes a 5′ universal primer and a 3′ universal primer;
a nucleotide sequence of the 5′ universal primer is shown in SEQ ID NO: 101; and
a nucleotide sequence of the 3′ universal primer is shown in SEQ ID NO: 102.

The present disclosure provides a method for amplifying an isomiR, including the following steps:
extracting total RNA from each of a gastric cancer sample and a non-gastric cancer sample, and reverse-transcribing the total RNA into cDNA;
with the cDNA as a template, conducting a first PCR preamplification using the primer set described above to obtain a first preamplification product;
with the first preamplification product as a template, conducting a second PCR preamplification using the transition primer and the reverse primer for amplifying the isomiR in the primer set described above to obtain a second preamplification product; and
with the second preamplification product as a template, conducting a third PCR preamplification using the 5′ universal primer and the 3′ universal primer in the primer set described above to obtain a third preamplification product, which is the isomiR.

The present disclosure provides a double unique dual indexing amplification primer set for construction of a high-throughput sample library for NGS, including primers for adding an inner double unique dual index (DUDI) and primers for adding an outer DUDI and a sequencing adapter,
where a forward primer among the primers for adding an inner DUDI is obtained by linking a DNA fragment shown in SEQ ID NO: 2055, an I5 Index sequence, and a DNA fragment shown in SEQ ID NO: 2056 sequentially;
a reverse primer among the primers for adding an inner DUDI is obtained by linking a DNA fragment shown in SEQ ID NO: 2057, an I7 Index sequence, and a DNA fragment shown in SEQ ID NO: 2058 sequentially;
a forward primer among the primers for adding an outer DUDI and a sequencing adapter is obtained by linking a DNA fragment shown in SEQ ID NO: 2059, the I5 Index sequence, and a DNA fragment shown in SEQ ID NO: 2060 sequentially;
a reverse primer among the primers for adding an outer DUDI and a sequencing adapter is obtained by linking a DNA fragment shown in SEQ ID NO: 2061, the I7 Index sequence, and a DNA fragment shown in SEQ ID NO: 2062 sequentially;
a nucleotide sequence of the I5 Index sequence is one selected from the group consisting of SEQ ID NO: 103 to SEQ ID NO: 1078; and
a nucleotide sequence of the I7 Index sequence is one selected from the group consisting of SEQ ID NO: 1079 to SEQ ID NO: 2054.

The present disclosure provides a kit for construction of a high-throughput sample library for NGS, including the primer set for amplification of an isomiR described above, the double unique dual indexing amplification primer set described above, and 2× boost mix,
where the 2× Boost mix includes the following components: water as a solvent, Tris-HCl: 70 mmol/L to 80 mmol/L, (NH₄)₂SO₄: 15 mmol/L to 25 mmol/L, Triton-100: 0.08% to 0.12% in a volume concentration, MgCl₂: 2 mmol/L to 3 mmol/L, dNTPs: 150 μmol/L to 250 μmol/L, trehalose: 190 mmol/L to 210 mmol/L, and hot-start Taq DNA polymerase: 45,000 U/L to 55,000 U/L; a pH of the Tris-HCl is 8.5 to 9.0; and
the dNTPs refers to a dNTP mixed solution that includes UDG and does not include dUTP.

The present disclosure provides a method for construction of a high-throughput sample library for NGS, including the following steps:
with the third preamplification product obtained by the method described above as a template, conducting a first PCR amplification using the primers for adding an inner DUDI in the double unique dual indexing amplification primer set described above to obtain an IUDI-containing PCR product;
with the IUDI-containing PCR product as a template, conducting a second PCR amplification using the primers for adding an outer DUDI and a sequencing adapter in the double unique dual indexing amplification primer set described above to obtain a DUDI-containing PCR product; and pooling to obtain a sequencing library.

The present disclosure provides a method for construction of a high-throughput sample library for NGS, including the following step:
with the third preamplification product obtained by the method described above as a template, conducting a PCR amplification using the double unique dual indexing amplification primer set described above to obtain a DUDI-containing PCR product.

Preferably, the method for construction of a high-throughput sample library for NGS further includes: precipitating a pooled DUDI-containing PCR product, and removing PCR primers from a product precipitate with an ExoI enzyme to obtain the sequencing library.

The present disclosure provides a use of the primer set for amplification of an isomiR described above, an isomiR amplified by the method described above, or the double unique dual indexing amplification primer set described above in construction of a prediction model for artificial intelligent diagnosis of a tumor based on NGS.

Preferably, the tumor includes gastric cancer.

The primer set for amplification of an isomiR provided in present disclosure includes a universal sequence and a 5′-terminus amplification primer linked sequentially to a partial sequence of a 5′ terminus of an isomiR. In the present disclosure, the universal sequence is added to a 3′ terminus, that is, sequences of 3′ termini of cDNAs of all miRNAs are the same. In the general traditional NGS, a 5′ terminus is miRNA-specific, that is, amplification primers for different miRNAs are different, but in order to amplify isoforms of a same miRNA, amplification primers for each miRNA are universal to isoforms of the miRNA, that is, amplification primers for a miRNA can be used to amplify all isoforms of the miRNA. For this reason, in the present disclosure, amplification primers for each miRNA are designed according to a universal sequence and a primer linked sequentially to a partial sequence of a 5′ terminus of an isomiR, and isomiRs can be successfully amplified with the amplification primers. As a most obvious advantage, the primers provided by the present disclosure can be selected according to a corresponding miRNA to be amplified. Thus, the primers have the following advantages: 1. High sensitivity: The primers provided in the present disclosure can be used to detect miRNAs that cannot be detected by the conventional methods. Because all miRNAs are detected in the conventional methods and concentrations of miRNAs may vary by a factor of several thousands, only miRNAs with relatively-high expression levels may be detected at a specified sequencing depth. However, the early diagnosis of tumors requires the detection of miRNAs at relatively-low concentrations, which obviously cannot be allowed by the current miRNA second-generation detection technologies. The method provided by the present disclosure can effectively solve this problem. 2. High relative sequencing depth: For amplification of the same low-expression miRNAs, due to significant amplification and avoidance of high-expression miRNAs, a relative sequencing depth of the technology with the primers of the present disclosure can be much higher than a relative sequencing depth of a conventional technology. 3. High specificity: Because an adapter is indiscriminately added to each of two termini of cDNA in the conventional technology, NGS data of the conventional technology include a large number of useless sequences in addition to miRNA sequences, such as tRNA and other small RNA sequences, resulting in a low efficiency. The primer set provided by the present disclosure can meet the requirements of specific amplification, and includes few useless sequences, resulting in a high efficiency.

The present disclosure provides a double unique dual indexing amplification primer set for construction of a high-throughput sample library for NGS, including primers for adding an inner DUDI and primers for adding an outer DUDI and a sequencing adapter, where a forward primer among the primers for adding an inner DUDI is obtained by linking a DNA fragment shown in SEQ ID NO: 2055, an I5 Index sequence, and a DNA fragment shown in SEQ ID NO: 2056 sequentially; a reverse primer among the primers for adding an inner DUDI is obtained by linking a DNA fragment shown in SEQ ID NO: 2057, an I7 Index sequence, and a DNA fragment shown in SEQ ID NO: 2058 sequentially; a forward primer among the primers for adding an outer DUDI and a sequencing adapter is obtained by linking a DNA fragment shown in SEQ ID NO: 2059, the I5 Index sequence, and a DNA fragment shown in SEQ ID NO: 2060 sequentially; a reverse primer among the primers for adding an outer DUDI and a sequencing adapter is obtained by linking a DNA fragment shown in SEQ ID NO: 2061, the I7 Index sequence, and a DNA fragment shown in SEQ ID NO: 2062 sequentially; a nucleotide sequence of the I5 Index sequence is one selected from the group consisting of SEQ ID NO: 103 to SEQ ID NO: 1078; and a nucleotide sequence of the I7 Index sequence is one selected from the group consisting of SEQ ID NO: 1079 to SEQ ID NO: 2054. The primer set includes an inner dual index and an outer dual index, which facilitates the subsequent addition of the indexes to a DNA fragment to be sequenced of an amplified sample through PCR amplification. Each primer includes a pair of specific base sequences (I5 Index or I7 Index), which is obtained as follows: 10 base-unique short sequences are randomly produced, complementary sequences are removed, and then a DUDI is screened out according to the following criteria: a same base should not be repeated three or more times; a sequence is not seriously complementary to other sequences; a sequence is at least 3 bases different from other sequences; after two sequences of a same DUDI bind to surrounding sequences, the specific amplification of the primer is not affected, that is, the possibility of producing a primer dimer is not increased; and a score of pairing between two sequences is calculated, and a pair with a minimum score (namely, maximum specificity) is selected as a barcode index added in a same sample. According to the above criteria, 976 pairs of DUDIs are screened out for high-throughput samples of NGS. In addition, because sequences of different DUDIs are at least 3 bases different from each other, the primers developed based on the double unique dual indexing technology still maintain their uniqueness and will not have other unique dual indexes even if there is a sequencing error and one base mismatch is allowed. Thus, the indexing has a very high accuracy. In terms of this advantage, the method of the present disclosure is also superior to the conventional method. Because a large number of sequences need to be indexed in the conventional method, for example, 2,000 sequences need to be indexed for 1,000 samples, a probability of false indexing of the conventional method is at least 5 times a probability of false indexing of the method of the present disclosure. In the present disclosure, a large number of samples are analyzed, and more than 400 G of data are acquired, but there is no mismatched NGS read.

In addition, when PCR amplification is conducted with the primers, a sequencing adapter and a barcode index sequence are added simultaneously. After a library is constructed in this way, each DNA molecule includes an OUDI and an IUDI. Through the combination of OUDIs and IUDIs in different quantities, a corresponding number of samples can be pooled. For example, if 1,000 samples need to be pooled for sequencing, the samples are first indexed with 200 pairs of IUDIs, where the 200 pairs of IUDIs can index the 1,000 samples in 5 groups, and then different OUDIs can be added to the 5 groups of samples each with the same unique dual indexes during library construction, such that the samples can be distinguished. With this simple double unique dual indexing technology, tens of thousands of samples can be specifically indexed and then pooled together for sequencing, which allows the pooled sequencing of any number of samples. The provision of the primers can greatly reduce the sequencing and primer costs. The double unique dual indexing technology adopts a multiply operation, while the traditional dual indexing technology adopts an addition operation. For example, if 205 pairs of primers are synthesized, 1,000 samples can be indexed by the double unique dual indexing technology, but only 205 samples can be indexed by the traditional dual indexing technology, and it is necessary to consider that one or two of the 205 pairs of indexes cannot be the same as indexes of other samples before loading for sequencing in the traditional dual indexing technology, which is troublesome and sometimes cannot be satisfied. With the primers developed based on the double unique dual indexing technology in the present disclosure, it is merely necessary to consider that one or two of the 5 pairs of outer indexes cannot be the same as indexes of other samples, which greatly reduces the possibility of an index conflict and allows the simultaneous sequencing of 1,000 samples. In addition, compared with the existing technologies, the double unique dual indexing technology can greatly reduce a cost of primer synthesis.

NGS-associated primers are relatively long and usually have a length of larger than 50 bp, and require NGS-grade purification, resulting in a high cost. The primers developed by the present disclosure can reduce a cost by at least 5 times, and can also simplify the split and increase the operability. In the method of the present disclosure, a plurality of samples are divided into several groups, for example, 1,000 samples are divided into 5 groups, and thus only 200 samples need to be split in each group. The method of the present disclosure, which involves only one large group but 1,000 samples, makes the computer splitting easier than the conventional method.

The present disclosure provides a use of the primer set for amplification of an isomiR described above, an isomiR amplified by the method described above, or the double unique dual indexing amplification primer set described above in construction of a prediction model for artificial intelligent diagnosis of a tumor based on NGS. In the present disclosure, NGS is conducted with a high-throughput sample isomiR library conducted based on the double unique dual indexing amplification primer set, and then a machine learning model is constructed based on NGS results. The machine learning model constructed has excellent prediction performance due to the excellent repeatability of the NGS results of the present disclosure. Experimental results show that two confusion matrices for mutual authentication both have an accuracy of 95% and a sensitivity and specificity of 90% or more, indicating that NGS data of the two times are highly similar, that is, the biological repeatability is high. NGS data of a third batch (a third confusion matrix) predicted by a second model also have an accuracy of 95% and a sensitivity and specificity of 90% or more, indicating that multiple times of NGS of a same sample have high biological repeatability. What is more important is whether the machine learning model constructed is universal, that is, whether the machine learning model can be used to predict NGS data of different samples. NGS data of another batch of completely different samples are successfully predicted by the second model above (a fourth confusion matrix). NGS data of a second batch of completely different samples are also successfully predicted by the same model (a fifth confusion matrix). While a confusion matrix has lower accuracy, sensitivity, and specificity than the prediction of NGS data of the same samples from different batches, it is expected, and given that a large amount of data is required for modeling by machine learning (because gastric cancer has high genetic heterogeneity), 200 samples are insufficient. However, importantly, P values of confusion matrices are low, indicating that prediction results are very statistically significant and cannot be coincidental.

These experimental results fully show that the prediction model for artificial intelligent diagnosis of a tumor based on NGS constructed in the present disclosure can effectively distinguish between gastric cancer and non-gastric cancer diseases (gastritis, gastric ulcer, gastric erosion, and other gastric discomforts), and has excellent biological repeatability (when different samples are adopted). The sensitivity and specificity of prediction by the prediction model both can reach 90% or more. It indicates that the double unique dual indexing technology for multiplex NGS and corresponding primers developed in the present disclosure have both high technical repeatability and high biological repeatability, and can detect a natural variation of a biological sample, that is, specific detection results. If a detection is not specific, a non-specific signal masks a specific signal, and thus it is impossible to obtain such a specific detection result. The above-mentioned NGS results prove from the technical repeatability and the biological repeatability that the NGS library construction technology for isomiRs developed in the present disclosure has high repeatability and can be used for artificial intelligent diagnosis of a tumor.

### BRIEF DESCRIPTION OF THE DRAWINGS

FIG. 1 is a schematic diagram of a principle of a DUDI technology for high-throughput samples of NGS;

FIG. 2 is a scatter plot of principal component analysis (PCA) for NGS results of three replicated batches;

FIG. 3 is a histogram of Silhouette scores of PCA for NGS results of three replicated batches; and

FIGS. 4A-4E show the comparison of confusion matrices for machine learning.

### DETAILED DESCRIPTION OF THE EMBODIMENTS

The present disclosure provides a primer set for amplification of an isomiR, including a universal sequence and a 5′-terminus amplification primer linked sequentially to a partial sequence of a 5′ terminus of a miRNA.

In the present disclosure, in order to allow the amplification of isomiRs, a universal sequence is sequentially linked to a partial sequence of a 5′ terminus of a target miRNA, which ensures both the specific amplification of a miRNA and the amplification of all different isoforms of a specific miRNA and also allows the flexibility of a test object. In an embodiment of the present disclosure, the universal sequence is ATAGACTCCTCGCATAGCCTCATGAGTC (SEQ ID NO: 2057). A length of the partial sequence of the 5′ terminus of the miRNA is preferably 12 nt to 14 nt and more preferably 13 nt.

In an embodiment of the present disclosure, in order to prove that the primer set provided in the present disclosure can allow the amplification of isomiRs, a miRNA associated with gastric cancer is illustrated as an example. The miRNA preferably includes at least one selected from the group consisting of the following: hsa-miR-21-5p, hsa-miR-223-3p, hsa-miR-223-5p, hsa-miR-186-5p, hsa-miR-18a-5p, hsa-miR-146b-5p, hsa-miR-624-5p, hsa-miR-106b-5p, hsa-miR-340-5p, hsa-miR-20a-5p, hsa-miR-451a, hsa-miR-7976, hsa-miR-2355-3p, hsa-miR-301a-3p, hsa-miR-144-5p, hsa-miR-151a-3p, hsa-miR-3200-5p, hsa-miR-1537-3p, hsa-miR-500a-5p, hsa-miR-127-3p, hsa-miR-570-3p, hsa-miR-130b-5p, hsa-miR-503-5p, hsa-miR-551a, hsa-miR-409-3p, hsa-miR-330-3p, hsa-miR-889-3p, hsa-miR-625-5p, hsa-miR-542-3p, hsa-miR-582-3p, hsa-miR-381-3p, hsa-miR-495-3p, hsa-miR-103a-1-5p, hsa-miR-450b-5p, hsa-miR-429, hsa-miR-576-5p, hsa-miR-148b-3p, hsa-miR-320c, hsa-miR-4286, hsa-miR-126-3p, hsa-miR-152-3p, hsa-miR-144-3p, hsa-miR-195-5p, hsa-let-7a-5p, hsa-miR-378f, hsa-miR-126-5p, hsa-miR-26a-5p, hsa-miR-29a-3p, hsa-miR-181a-5p, hsa-miR-32-5p, hsa-miR-142-3p, hsa-miR-29c-3p, hsa-miR-424-5p, hsa-miR-192-5p, hsa-miR-143-3p, hsa-miR-30c-5p, hsa-miR-146a-5p, hsa-miR-101-3p, hsa-miR-19b-3p, hsa-miR-33b-5p, hsa-miR-378a-3p, hsa-miR-22-3p, hsa-miR-107, hsa-miR-497-5p, hsa-miR-15a-3p, hsa-miR-188-5p, hsa-let-7d-3p, hsa-miR-132-3p, hsa-miR-151a-5p, hsa-miR-194-5p, hsa-miR-99a-5p, hsa-miR-125b-5p, hsa-miR-25-3p, hsa-miR-103a-3p, hsa-miR-1285-3p, hsa-miR-7977, hsa-miR-30b-5p, hsa-miR-363-3p, hsa-miR-93-5p, hsa-miR-375-3p, hsa-miR-99b-5p, hsa-miR-193b-3p, hsa-miR-324-3p, hsa-miR-193a-3p, hsa-miR-342-3p, hsa-miR-484, hsa-miR-532-3p, hsa-miR-210-3p, hsa-miR-2110, hsa-miR-296-5p, hsa-miR-1307-5p, hsa-miR-19a-3p, hsa-miR-139-5p, hsa-miR-3665, hsa-miR-RG-84, hsa-miR-4454, and hsa-let-7b-5p; and according to the order of the miRNAs, nucleotide sequences of 5′-terminus amplification primers designed correspondingly are shown in SEQ ID NO: 1 to SEQ ID NO: 97, respectively.

In the present disclosure, the primer set preferably further includes a transition primer and a reverse primer for amplifying the isomiR; a nucleotide sequence of the transition primer is shown in SEQ ID NO: 99 (TCTACAGATCCTGGCCTCTGACTCCAGGATCTGTAGAC

CTCCATCCGAGACACACGAT); and a nucleotide sequence of the reverse primer for amplifying the isomiR is shown in SEQ ID NO: 100 (GTTTGTTGCTACGCTCAGAATCCTAAGCGTAGCAACAAACATAGACTCCTCGCATAGCC TCATGAGTC).

In the present disclosure, the primer set preferably further includes a 5′ universal primer and a 3′ universal primer. A nucleotide sequence of the 5′ universal primer is shown in SEQ ID NO: 101 (CAGAATCCTAAGCGTAGCAACAAAC); and a nucleotide sequence of the 3′ universal primer is shown in SEQ ID NO: 102 (GCCTCTGACTCCAGGATCTGTAGAC).

The present disclosure has no special restrictions on sources of the primers, and the primers can be synthesized by a gene synthesis method well known in the art.

The present disclosure provides a method for amplifying an isomiR, including the following steps:
total RNA is extracted from each of a gastric cancer sample and a non-gastric cancer sample, and reverse-transcribed into cDNA;
with the cDNA as a template, a first PCR preamplification is conducted using the primer set described above to obtain a first preamplification product;
with the first preamplification product as a template, a second PCR preamplification is conducted using the transition primer and the reverse primer for amplifying the isomiR in the primer set described above to obtain a second preamplification product; and
with the second preamplification product as a template, a third PCR preamplification is conducted using the 5′ universal primer and the 3′ universal primer in the primer set described above to obtain a third preamplification product, which is the isomiR.

In the present disclosure, total RNA is extracted from each of a gastric cancer sample and a non-gastric cancer sample, and reverse-transcribed into cDNA.

The present disclosure has no special restrictions on a method for extracting the total RNA, and a method for extracting total RNA well known in the art may be adopted. For example, a commercial kit method can be used to extract the total RNA.

In the present disclosure, the reverse-transcription includes a PolyA reaction, a denaturation reaction, and a reverse-transcription reaction. A system for the PolyA reaction is preferably of 20 μL, and includes the following reagents: 5× reverse-transcription buffer: 4 μL, 10 mM ATP: 2 μL, 5,000 U/μl PolyA enzyme: 1 μL, 40,000 U/μl RNA Inhibitor: 0.5 μL, and RNA sample: 12.5 μL. The PolyA reaction is preferably conducted at 37° C. for 30 min and then at 65° C. for 20 min. A system for the denaturation reaction is preferably of 20 μL, and includes the following reagents: 10 mM dNTPs: 1.5 μL, 10 μM reverse-transcription primer (USRTPn): 1.5 μL, and Poly A reaction product: 17 μL. The reverse-transcription primer is preferably USRTPn with a corresponding nucleotide sequence shown in SEQ ID NO: 2063 (CCTCCATCCGAGACACACGATTGATGGTTTTTTTTTTTTTTTTTTVN). The denaturation reaction is preferably conducted as follows: the system for the denaturation reaction is heated at 65° C. for 5 min, then taken out 1 s before the end of the heating, and then immediately incubated in an ice bath for 1 min. A system for the reverse-transcription reaction is preferably of 30 μL, and includes the following reagents: 5× reverse-transcription buffer: 2 μL, 1.6 M trehalose: 4.5 μL, 1 mg/μL Actinomycin D: 1.2 μL, T4gp32/RecA/ATP mixed solution: 1.5 μL, 40,000 U/μL RNA Inhibitor: 0.3 μL, 50 U/μL Maxima H reverse transcriptase: 1.5 μL, and denaturation reaction product: 19 μL. Based on one sample, the T4gp32/RecA/ATP mixed solution is preferably prepared from the following reagents: 10 μg/μL T4gp32: 0.6 μL, 2 μg/μL Tth RecA: 0.2 μL, 100 mM ATP: 0.24 μL, and 1× reverse-transcription buffer: 1.96 μL. The reverse-transcription reaction is preferably conducted at 42° C. for 15 min, 50° C. for 30 min. 55° C. for 30 min, 60° C. for 30 min, 65° C. for 30 min, and then 85° C. for 5 min.

In the present disclosure, after the cDNA is obtained, with the cDNA as a template, a first PCR preamplification is conducted using the primer set described above to obtain a first preamplification product.

In the present disclosure, a reaction system for the first PCR preamplification is preferably of 20 μL, and includes the following reagents: 2× Boost mix: 10 μL, 0.2 μg/μl Tth RecA: 1 μL, 1 μM primer set: 1.5 μL, and cDNA: 7.5 μL. A composition and a preparation method of 2× Boost mix can specifically refer to a specific quantitative PCR reaction mixed solution in Example 1 recorded in the patent ZL 201910219827.4 “Specific Quantitative PCR Mixed Solution, miRNA Quantitative Detection Kit, and Detection Method”, but the 2× Boost mix (including UDG) is prepared with a dNTP mixed solution without dUTP. A reaction procedure for the first PCR preamplification is preferably as follows: (1) 25° C. for 10 min; (2) 95° C. for 10 min; (3) 3 cycles of (95° C. for 10 s and 55° C. for 10 min); (4) 3 cycles of (95° C. for 10 s and 50° C. for 10 min); (5) 2 cycles of (95° C. for 10 s and 45° C. for 10 min); (6) 2 cycles of (95° C. for 10 s and 40° C. for 10 min); (7) 2 cycles of (95° C. for 10 s and 37° C. for 10 min); (8) 1 cycle of (95° C. for 10 s, 60° C. for 2 min, and 72° C. for 10 min); and (9) a PCR tube is incubated at 72° C. for 5 min, and then taken out and immediately incubated in an ice bath. The first PCR preamplification facilitates the amplification of a large number of isomiRs from reverse-transcription products. The first preamplification product is purified and then treated with an EXO I enzyme to remove PCR primers from the system.

In the present disclosure, after the first preamplification product is obtained, with the first preamplification product as a template, a second PCR preamplification is conducted using the transition primer and the reverse primer for amplifying the isomiR in the primer set described above to obtain a second preamplification product.

In the present disclosure, a reaction system for the second PCR preamplification is preferably of 20 μL, and includes the following reagents: 2× Boost mix: 10 μL, 10 μm transition primer (USEXPnb): 1 μL, 10 μm isomiR primer: 1 μL, 0.2 μg/μL Tth RecA: 1 μL, and first preamplification product: 7 μL. A reaction procedure for the second PCR preamplification is preferably as follows: (1) 25° C. for 10 min; (2) 95° C. for 10 min; (3) 3 cycles of (95° C. for 10 s and 65° C. for 10 min); (4) 3 cycles of (95° C. for 10 s and 62° C. for 10 min); (5) 2 cycles of (95° C. for 10 s and 58° C. for 2 min); (6) 2 cycles of (95° C. for 10 s and 60° C. for 2 min); (7) 1 cycle of (95° C. for 10 s, 60° C. for 2 min, and 72° C. for 10 min); and (8) a PCR tube is incubated at 72° C. for 5 min, and then taken out and incubated in an ice bath. The second preamplification product is preferably purified with magnetic beads. The second PCR pre-amplification is conducted with the transition primer and the reverse primer for amplifying the isomiR, and is intended to introduce binding sites for the 3′ universal primer and the 5′ universal primer.

With the second preamplification product as a template, a third PCR preamplification is conducted using the 5′ universal primer and the 3′ universal primer in the primer set described above to obtain a third preamplification product, which is the isomiR.

In the present disclosure, a reaction system for the third PCR preamplification is preferably of 20 μL, and includes the following reagents: 2× Boost mix: 10 μL, 10 μm URP: 1 μL, 10 μm UFP: 1 μL, 0.2 μg/μL Tth RecA: 1 μL, and second preamplification product: 7 μL. A reaction procedure for the third PCR preamplification is preferably as follows: (1) 95° C. for 10 min; (2) 12 cycles of (95° C. for 10 s and 65° C. for 1 min); (4) 72° C. for 10 min; and (5) 72° C. for 5 min and then incubation in an ice bath. The third preamplification product is purified and then treated with an EXO I enzyme to remove PCR primers.

In the present disclosure, after the third preamplification product is obtained, qPCR amplification is conducted preferably with the third preamplification product as a template to obtain an expression level of the isomiR as a part of quality control. A forward primer for the qPCR amplification is preferably a 5′ universal primer. A reverse primer for the qPCR amplification is preferably a 3′ universal primer. A probe for the qPCR amplification is preferably an LNAFAM probe, and a corresponding nucleotide sequence of the probe is shown in SEQ ID NO: 2064 (ACC+AT+CA+AT+CG+TG+TG, where + represents a locked nucleic acid (LNA)). A reaction system for the qPCR amplification is preferably of 10 μL, and preferably includes the following reagents: fold-diluted third preamplification product: 0.08 μL, 2× DNA polymerase mixture: 5 μL, forward primer with a final concentration of 0.2 μM, reverse primer with a final concentration of 0.2 μM, probe with a final concentration of 0.2 μM, and ddH₂O: making up to 10 μL. A reaction procedure for the qPCR amplification is preferably as follows: 95° C. for 10 min, 95° C. for 30 s, and 65° C. for 1 min, with 40 cycles.

The present disclosure provides a double unique dual indexing amplification primer set for construction of a high-throughput sample library for NGS, including primers for adding an inner DUDI and primers for adding an outer DUDI and a sequencing adapter, where a forward primer among the primers for adding an inner DUDI is obtained by linking a DNA fragment shown in SEQ ID NO: 2055 (CACGACGCTCTTCCGATCT), an I5 Index sequence, and a DNA fragment shown in SEQ ID NO: 2056 (CAAACATAGACTCCTCGCATAGCCT) sequentially; a reverse primer among the primers for adding an inner DUDI is obtained by linking a DNA fragment shown in SEQ ID NO: 2057 (CTCGGAGATGTGTATAAGAGACAG), an I7 Index sequence, and a DNA fragment shown in SEQ ID NO: 2058 (ACCTCCATCCGAGACACACG) sequentially; a forward primer among the primers for adding an outer DUDI and a sequencing adapter is obtained by linking a DNA fragment shown in SEQ ID NO: 2059 (AATGATACGGCGACCACCGAGATCTACAC), the I5 Index sequence, and a DNA fragment shown in SEQ ID NO: 2060 (ACACTCTTTCCCTACACGACGCTCTTCCGATCT) sequentially; a reverse primer among the primers for adding an outer DUDI and a sequencing adapter is obtained by linking a DNA fragment shown in SEQ ID NO: 2061 (CTGTCTCTTATACACATCTCCGAGCCCACGAGAC), the I7 Index sequence, and a DNA fragment shown in SEQ ID NO: 2062 (CTCGGAGATGTGTATAAGAGACAG) sequentially; a nucleotide sequence of the I5 Index sequence is one selected from the group consisting of SEQ ID NO: 103 to SEQ ID NO: 1078; and a nucleotide sequence of the I7 Index sequence is one selected from the group consisting of SEQ ID NO: 1079 to SEQ ID NO: 2054.

In the present disclosure, the I5 Index sequence and the I7 Index sequence are combined into a set. The I5 Index sequence and the I7 Index sequence are preferably screened out as follows: 10 base-unique short sequences are randomly produced, complementary sequences are removed, and then DUDI is screened out preferably according to the following criteria: a same base should not be repeated three or more times; a sequence is not seriously complementary to other sequences; a sequence is at least 3 bases different from other sequences; after two sequences of a same DUDI bind to surrounding sequences, the specific amplification of the primer is not affected, that is, the possibility of producing a primer dimer is not increased; and a score of pairing between two sequences is calculated, and a pair with a minimum score (namely, maximum specificity) is selected as a set indexes for indexing forward and reverse primers. A total of 976 pairs of DUDIs are screened out for indexing high-throughput samples of NGS. Because sequences of different DUDIs are at least 3 bases different from each other, these indexes can still maintain their uniqueness and will not become other unique dual indexes even if there is a sequencing error and one base mismatch is allowed. Thus, the indexing has a very high accuracy. In terms of this advantage, the method of the present disclosure is also very superior to the conventional method. Because a large number of sequences need to be indexed in the conventional method, for example, 2,000 sequences need to be indexed for 1,000 samples, a probability of false indexing of the conventional method is at least 5 times a probability of false indexing of the method of the present disclosure. In the present disclosure, a large number of samples are analyzed through experiments, and more than 400 G of data are acquired, but there is no mismatched NGS read.

The present disclosure provides a kit for construction of a high-throughput sample library for NGS, including the primer set for amplification of an isomiR described above, the double unique dual indexing amplification primer set described above, and 2× boost mix, where the 2× Boost mix includes the following components: water as a solvent, Tris-HCl: 70 mmol/L to 80 mmol/L, (NH₄)₂SO₄: 15 mmol/L to 25 mmol/L, Triton-100: 0.08% to 0.12% in a volume concentration, MgCl₂: 2 mmol/L to 3 mmol/L, dNTPs: 150 μmol/L to 250 μmol/L, trehalose: 190 mmol/L to 210 mmol/L, and hot-start Taq DNA polymerase: 45,000 U/L to 55,000 U/L; a pH of the Tris-HCl is 8.5 to 9.0; and the dNTPs refers to a dNTP mixed solution that includes UDG and does not include dUTP.

The present disclosure provides a method for construction of a high-throughput sample library for NGS, including the following step:
with the third preamplification product obtained by the method described above as a template, a first PCR amplification is conducted using the primers for adding an inner DUDI in the double unique dual indexing amplification primer set described above to obtain an IUDI-containing PCR product;
with the IUDI-containing PCR product as a template, a second PCR amplification is conducted using the primers for adding an outer DUDI and a sequencing adapter in the double unique dual indexing amplification primer set described above to obtain a DUDI-containing PCR product; and pooling is conducted to obtain a sequencing library.

In the present disclosure, a reaction system for the first PCR amplification is preferably of 30 μL, and includes the following reagents: 2× PCR enzyme (including UDG and UTP): 15 μL, 10 μM each of forward and reverse primers for adding an inner DUDI: 0.5 μL, third preamplification product: 2 μL, and water: the balance. A reaction procedure for the first PCR amplification is preferably as follows: (1) 95° C. for 10 min: (2) 3 cycles of (95° C. for 15 s, 62° C. for 30 s, and 72° C. for 1 min); (3) 2 cycles of (95° C. for 15 s, 64° C. for 30 s, and 72° C. for 1 min); (4) 11 cycles of (95° C. for 15 s, 68° C. for 30 s, and 72° C. for 1 min); (5) 1 cycle of (95° C. for 15 s and 72° C. for 20 min); and (6) incubation at 72° C. for 18 min and then in an ice bath.

In the present disclosure, a reaction system for the second PCR amplification is preferably of 30 μL, and includes the following reagents: 2× PCR enzyme (including UDG and UTP): 15 μL, 10 μM each of forward and reverse primers for adding an inner DUDI: 0.5 μL, third preamplification product: 2 μL, and water: the balance. A reaction procedure for the second PCR amplification is preferably as follows: (1) 95° C. for 10 min; (2) 3 cycles of (95° C. for 15 s, 62° C. for 30 s, and 72° C. for 1 min); (3) 2 cycles of (95° C. for 15 s, 64° C. for 30 s, and 72° C. for 1 min); (4) 11 cycles of (95° C. for 15 s, 68° C. for 30 s, and 72° C. for 1 min); (5) 1 cycle of (95° C. for 15 s and 72° C. for 20 min); and (6) incubation at 72° C. for 18 min and then in an ice bath.

In the present disclosure, the method for construction of a high-throughput sample library for NGS preferably further includes: a pooled DUDI-containing PCR product is precipitated and treated with an ExoI enzyme to remove PCR primers to obtain the sequencing library. The removal of PCR primers refers to the removal of double unique dual indexing amplification primers including forward and reverse primers that do not react during the above PCR processes. The removal of PCR primers is intended to prevent downstream sequencing reactions of the PCR primers.

In the present disclosure, the DUDI-containing PCR product is obtained based on the double unique dual indexing technology for multiplex NGS developed in the present disclosure. In the double unique dual indexing technology for multiplex NGS, with cDNA as a template, an IUDI is added to each of two termini of the cDNA through PCR, and then an OUDI and a sequencing adapter are added to each of two termini of a PCR product obtained previously through PCR amplification to obtain a DUDI-carried PCR product; and DUDI-carried PCR products are pooled and subjected to PCR primer removal to obtain an amplification library for NGS analysis. After NGS is completed, original NGS data are split into a number of samples corresponding to the sample pooling according to DUDI sequences, and isomiRs are identified and quantified by removing irrelevant sequences.

The present disclosure provides a use of the primer set for amplification of an isomiR described above, an isomiR amplified by the method described above, or the double unique dual indexing amplification primer set described above in construction of a prediction model for artificial intelligent diagnosis of a tumor based on NGS.

In the present disclosure, the tumor preferably includes gastric cancer. In the present disclosure, it is determined by optimizing a classifier that a machine learning model for auxiliary diagnosis of gastric cancer is established with a support vector machine (SVM) algorithm. Preferably, parameters of the SVM algorithm are optimized through grid search, and numerical ranges of the parameters are as follows: gamma=2⁽⁻⁸⁻¹⁾ and cost=2⁽⁰⁻⁴⁾. A prediction model is validated through 10-fold cross-validation. Once a prediction model is obtained, the prediction model is further preferably evaluated. Criteria for the evaluation include accuracy and Kappa. Accuracy, Kappa, and other evaluation indexes are preferably described by a confusion matrix and a receiver operating characteristic (ROC) curve.

In an embodiment of the present disclosure, a prediction model for artificial intelligent diagnosis of a tumor based on NGS is constructed based on an optimized SVM algorithm and optimized parameters thereof with sequencing results obtained through comprehensive amplification of gastric cancer-associated isomiRs, library construction, and NGS as data. In the experiments of the present disclosure, the sequencing of a same sample is repeated three or more times, and sequencing data of different batches are used to build a model for predicting sequencing data of other batches. Sequencing results of the three or more times show high repeatability, indicating that NGS data based on isomiRs of high-throughput samples can be used in construction of a machine learning prediction model to allow the artificial intelligent auxiliary diagnosis of a tumor.

The reproducible double unique dual indexing library construction method for NGS of an isomiR and the use thereof provided by the present disclosure are described in detail below with reference to examples, but these examples may not be understood as a limitation to the protection scope of the present disclosure.

EXAMPLE 1

### An NGS Library Construction Method for isomiRs Derived from Gastric Cancer Samples

### I. Extraction of Sample RNA and Reverse Transcription and Preamplification of IsomiRs

### 1. Extraction of Sample RNA

Sample source description: Gastric cancer samples (300) and non-gastric cancer clinical samples (300) were collected from the Cancer Hospital Chinese Academy of Medical Sciences, the Beijing Cancer Hospital, the Second People's Hospital of Dongying, and the PKUCare Luzhong Hospital.

An RNA extraction kit (purchased from Thermo Fisher) was used to extract RNA from each of the gastric cancer samples and non-gastric cancer clinical samples, and specific operations were completed according to instructions of the RNA extraction kit. After RNA of each sample was extracted, total RNA with a qualified concentration and quality determined by a nucleic acid quantification detector was stored at −20° C. for later use.

### 2. Reverse-Transcription and Preamplification of isomiRs in Gastric Cancer Samples

### A. Poly A Reaction

For one sample, a 20 μL reaction system was prepared specifically from the following reagents: 5× reverse-transcription buffer: 4 μL, ATP (10 mM): 2 μL, PolyA enzyme (5,000 U/μL): 1 μL, RNA Inhibitor (40,000 U/μL): 0.5 μL, and total RNA: 12.5 μL.

The prepared reaction system was subjected to the PolyA reaction at 37° C. for 30 min and then at 65° C. for 20 min. A resulting reaction system was sealed with a film and then stored at −5° C. (for thermal inactivation).

Notes: a. When there are a plurality of samples, a total system is prepared first and then dispensed into each PCR tube, and then an RNA sample is added to each PCR tube. b. Each tube is labeled first, and then an RNA sample is added according to a label of a tube, where the label should be checked to determine whether the label is consistent with the sample.

### B. Reverse-Transcription Reaction

For a sample, a 20 μL reaction system was prepared specifically from the following reagents: 10 mM dNTPs: 1.5 μL, 10 μM reverse-transcription primer (USRTPn, CCTCCATCCGAGACACACGATTGATGGTTTTTTTTTTTTTTTTTTVN, SEQ ID NO: 2063): 1.5 μL, and Poly A template: 17 μL.

The prepared reaction system was heated at 65° C. for 5 min to allow a denaturation reaction, then taken out 1 s before the end of the heating and immediately incubated in an ice bath for 1 min, and then centrifuged.

*Notes: 1. The dNTPs here do not include dUTP, otherwise a reverse-transcription product of cDNA will be degraded.

2. A Master Mix method is always used to avoid a sampling quantity of less than or equal to 1 μL, the same below.

3. The USEXPnb and the IsomiRupb primer below need to be purified with magnetic beads.

For a sample, a 30 μL reaction system for the reverse-transcription reaction was prepared specifically from the following reagents: 5× reverse-transcription buffer: 2 μL, 1.6 M trehalose: 4.5 μL, Actinomycin D (1 mg/μL): 1.2 μL, T4gp32/RecA/ATP mixed solution: 1.5 μL, RNA Inhibitor (40,000 U/μL): 0.3 μL, Maxima H reverse transcriptase (50 U/μL): 1.5 μL, and denaturation reaction product: 19 μL.

For a sample, the T4gp32/RecA/ATP mixed solution was prepared from the following reagents: T4gp32 (10 μg/μL): 0.6 μL, Tth RecA (2 μg/μL): 0.2 μL, ATP (100 mM): 0.24 μL, and 1× reverse-transcription buffer: 1.96 μL.

The prepared reaction system was subjected to the reverse-transcription reaction at 42° C. for 15 min, 50° C. for 30 min, 55° C. for 30 min, 60° C. for 30 min, 65° C. for 30 min, and 85° C. for 5 min.

### C. Preamplification

### First PCR Preamplification

1. For a sample, a 20 μL reaction system for the first PCR preamplification was prepared from the following reagents: 2× Boost mix*: 10 μL, Tth RecA (0.2 μg/μL)**: 1 μL, Pre-IsomiR mix* (1 μM): 1.5 μL, and reverse-transcription product: 7.5 μL.

The 2× Boost mix* (including UDG) was prepared with a dNTP mixed solution without dUTP. The 2× Boost mix could specifically refer to a specific quantitative PCR reaction mixed solution in Example 1 of the patent ZL 201910219827.4 “Specific Quantitative PCR Mixed Solution, miRNA Quantitative Detection Kit, and Detection Method”.

Pre-IsomiR mix* (1 μM): 10 μL of each of 97 primers (a concentration of a primer stock solution was 100 μM, and specific sequences could be seen in Table 1) was taken, and then 20 μL of H₂O (Nuclease-Free) was added to prepare a primer mix with a final concentration of 1 μm (1,000 μL).

TABLE 1
5′-terminus amplification primers for isomiRs

| | | |
|---|---|---|
| hsa-miR-21-5p | ATAGACTCCTCGCATAGCCTCATGAGTCTAGCTTATCAGAC | SEQ ID NO: 1 |
| hsa-miR-223-3p | ATAGACTCCTCGCATAGCCTCATGAGTCTGTCAGTTTGTC | SEQ ID NO: 2 |
| hsa-miR-223-5p | ATAGACTCCTCGCATAGCCTCATGAGTCCGTGTATTTGAC | SEQ ID NO: 3 |
| hsa-miR-186-5p | ATAGACTCCTCGCATAGCCTCATGAGTCCAAAGAATTCTCC | SEQ ID NO: 4 |
| hsa-miR-18a-5p | ATAGACTCCTCGCATAGCCTCATGAGTCTAAGGTGCATCT | SEQ ID NO: 5 |
| hsa-miR-146b-5p | ATAGACTCCTCGCATAGCCTCATGAGTCTGAGAACTGAATTC | SEQ ID NO: 6 |
| hsa-miR-624-5p | ATAGACTCCTCGCATAGCCTCATGAGTCTAGTACCAGTACC | SEQ ID NO: 7 |
| hsa-miR-106b-5p | ATAGACTCCTCGCATAGCCTCATGAGTCTAAAGTGCTGAC | SEQ ID NO: 8 |
| hsa-miR-340-5p | ATAGACTCCTCGCATAGCCTCATGAGTCTTATAAAGCAATGAG | SEQ ID NO: 9 |
| hsa-miR-20a-5p | ATAGACTCCTCGCATAGCCTCATGAGTCTAAAGTGCTTATAG | SEQ ID NO: 10 |
| hsa-miR-45la | ATAGACTCCTCGCATAGCCTCATGAGTCTGCCCTGAGAC | SEQ ID NO: 11 |
| hsa-miR-7976 | ATAGACTCCTCGCATAGCCTCATGAGTCATTGTCCTTGC | SEQ ID NO: 12 |
| hsa-miR-2355-3p | ATAGACTCCTCGCATAGCCTCATGAGTCCAGTGCAATAGT | SEQ ID NO: 13 |
| hsa-miR-301a-3p | ATAGACTCCTCGCATAGCCTCATGAGTCGGATATCATCATATAC | SEQ ID NO: 14 |
| hsa-miR-144-5p | ATAGACTCCTCGCATAGCCTCATGAGTCCTAGACTGAAGC | SEQ ID NO: 15 |
| hsa-miR-151a-3p | ATAGACTCCTCGCATAGCCTCATGAGTCAATCTGAGAAGGC | SEQ ID NO: 16 |
| hsa-miR-3200-5p | ATAGACTCCTCGCATAGCCTCATGAGTCAAAACCGTCTAGT | SEQ ID NO: 17 |
| hsa-miR-1537-3p | ATAGACTCCTCGCATAGCCTCATGAGTCTAATCCTTGCTAC | SEQ ID NO: 18 |
| hsa-miR-500a-5p | ATAGACTCCTCGCATAGCCTCATGAGTCTCGGATCCGT | SEQ ID NO: 19 |
| hsa-miR-127-3p | ATAGACTCCTCGCATAGCCTCATGAGTCCGAAAACAGCAAT | SEQ ID NO: 20 |
| hsa-miR-570-3p | ATAGACTCCTCGCATAGCCTCATGAGTCACTCTTTCCCTG | SEQ ID NO: 21 |
| hsa-miR-130b-5p | ATAGACTCCTCGCATAGCCTCATGAGTCTAGCAGCGGG | SEQ ID NO: 22 |
| hsa-miR-503-5p | ATAGACTCCTCGCATAGCCTCATGAGTCGCGACCCAC | SEQ ID NO: 23 |
| hsa-miR-55la | ATAGACTCCTCGCATAGCCTCATGAGTCGAATGTTGCTCG | SEQ ID NO: 24 |
| hsa-miR-409-3p | ATAGACTCCTCGCATAGCCTCATGAGTCGCAAAGCACAC | SEQ ID NO: 25 |
| hsa-miR-330-3p | ATAGACTCCTCGCATAGCCTCATGAGTCTTAATATCGGACAAC | SEQ ID NO: 26 |
| hsa-miR-889-3p | ATAGACTCCTCGCATAGCCTCATGAGTCAGGGGGAAAGT | SEQ ID NO: 27 |
| hsa-miR-625-5p | ATAGACTCCTCGCATAGCCTCATGAGTCTGTGACAGATTG | SEQ ID NO: 28 |
| hsa-miR-542-3p | ATAGACTCCTCGCATAGCCTCATGAGTCTAACTGGTTGAACAAC | SEQ ID NO: 29 |
| hsa-miR-582-3p | ATAGACTCCTCGCATAGCCTCATGAGTCTATACAAGGGCAAG | SEQ ID NO: 30 |
| hsa-miR-381-3p | ATAGACTCCTCGCATAGCCTCATGAGTCAAACAAACATGG | SEQ ID NO: 31 |
| hsa-miR-495-3p | ATAGACTCCTCGCATAGCCTCATGAGTCGGCTTCTTTACAG | SEQ ID NO: 32 |
| hsa-miR-103a-1-5p | ATAGACTCCTCGCATAGCCTCATGAGTCTTTTGCAATATGT | SEQ ID NO: 33 |
| hsa-miR-450b-5p | ATAGACTCCTCGCATAGCCTCATGAGTCTAATACTGTCTGG | SEQ ID NO: 34 |
| hsa-miR-429 | ATAGACTCCTCGCATAGCCTCATGAGTCATTCTAATTTCTCC | SEQ ID NO: 35 |
| hsa-miR-576-5p | ATAGACTCCTCGCATAGCCTCATGAGTCTCAGTGCATCAC | SEQ ID NO: 36 |
| hsa-miR-148b-3p | ATAGACTCCTCGCATAGCCTCATGAGTCAAAAGCTGGGT | SEQ ID NO: 37 |
| hsa-miR-320c | ATAGACTCCTCGCATAGCCTCATGAGTCACCCCACTCC | SEQ ID NO: 38 |
| hsa-miR-4286 | ATAGACTCCTCGCATAGCCTCATGAGTCTCGTACCGTG | SEQ ID NO: 39 |
| hsa-miR-126-3p | ATAGACTCCTCGCATAGCCTCATGAGTCTCAGTGCATGAC | SEQ ID NO: 40 |
| hsa-miR-152-3p | ATAGACTCCTCGCATAGCCTCATGAGTCTACAGTATAGATGAT | SEQ ID NO: 41 |
| hsa-miR-144-3p | ATAGACTCCTCGCATAGCCTCATGAGTCTAGCAGCACAG | SEQ ID NO: 42 |
| hsa-miR-195-5p | ATAGACTCCTCGCATAGCCTCATGAGTCTGAGGTAGTAGG | SEQ ID NO: 43 |
| hsa-let-7a-5p | ATAGACTCCTCGCATAGCCTCATGAGTCACTGGACTTGG | SEQ ID NO: 44 |
| hsa-miR-378f | ATAGACTCCTCGCATAGCCTCATGAGTCCATTATTACTTTTGG | SEQ ID NO: 45 |
| hsa-miR-126-5p | ATAGACTCCTCGCATAGCCTCATGAGTCTTCAAGTAATCCAG | SEQ ID NO: 46 |
| hsa-miR-26a-5p | ATAGACTCCTCGCATAGCCTCATGAGTCTAGCACCATCTG | SEQ ID NO: 47 |
| hsa-miR-29a-3p | ATAGACTCCTCGCATAGCCTCATGAGTCAACATTCAACGC | SEQ ID NO: 48 |
| hsa-miR-181a-5p | ATAGACTCCTCGCATAGCCTCATGAGTCTATTGCACATTAC | SEQ ID NO: 49 |
| hsa-miR-32-5p | ATAGACTCCTCGCATAGCCTCATGAGTCTGTAGTGTTTCC | SEQ ID NO: 50 |
| hsa-miR-142-3p | ATAGACTCCTCGCATAGCCTCATGAGTCTAGCACCATTTG | SEQ ID NO: 51 |
| hsa-miR-29c-3p | ATAGACTCCTCGCATAGCCTCATGAGTCCAGCAGCAATTC | SEQ ID NO: 52 |
| hsa-miR-424-5p | ATAGACTCCTCGCATAGCCTCATGAGTCCTGACCTATGAAT | SEQ ID NO: 53 |
| hsa-miR-192-5p | ATAGACTCCTCGCATAGCCTCATGAGTCTGAGATGAAGCAC | SEQ ID NO: 54 |
| hsa-miR-143-3p | ATAGACTCCTCGCATAGCCTCATGAGTCTGTAAACATCCTA | SEQ ID NO: 55 |
| hsa-miR-30c-5p | ATAGACTCCTCGCATAGCCTCATGAGTCTGAGAACTGAATTC | SEQ ID NO: 56 |
| hsa-miR-146a-5p | ATAGACTCCTCGCATAGCCTCATGAGTCTACAGTACTGTGAT | SEQ ID NO: 57 |
| hsa-miR-101-3p | ATAGACTCCTCGCATAGCCTCATGAGTCTGTGCAAATCC | SEQ ID NO: 58 |
| hsa-miR-19b-3p | ATAGACTCCTCGCATAGCCTCATGAGTCGTGCATTGCTG | SEQ ID NO: 59 |
| hsa-miR-33b-5p | ATAGACTCCTCGCATAGCCTCATGAGTCACTGGACTTGG | SEQ ID NO: 60 |
| hsa-miR-378a-3p | ATAGACTCCTCGCATAGCCTCATGAGTCAAGCTGCCAGT | SEQ ID NO: 61 |
| hsa-miR-22-3p | ATAGACTCCTCGCATAGCCTCATGAGTCAGCAGCATTGT | SEQ ID NO: 62 |
| hsa-miR-107 | ATAGACTCCTCGCATAGCCTCATGAGTCCAGCAGCAC | SEQ ID NO: 63 |
| hsa-miR-497-5p | ATAGACTCCTCGCATAGCCTCATGAGTCCAGGCCATATTG | SEQ ID NO: 64 |
| hsa-miR-15a-3p | ATAGACTCCTCGCATAGCCTCATGAGTCCATCCCTTGCAT | SEQ ID NO: 65 |
| hsa-miR-188-5p | ATAGACTCCTCGCATAGCCTCATGAGTCCTATACGACCTG | SEQ ID NO: 66 |
| hsa-let-7d-3p | ATAGACTCCTCGCATAGCCTCATGAGTCTAACAGTCTACAG | SEQ ID NO: 67 |
| hsa-miR-132-3p | ATAGACTCCTCGCATAGCCTCATGAGTCTCGAGGAGCTC | SEQ ID NO: 68 |
| hsa-miR-151a-5p | ATAGACTCCTCGCATAGCCTCATGAGTCTGTAACAGCAAC | SEQ ID NO: 69 |
| hsa-miR-194-5p | ATAGACTCCTCGCATAGCCTCATGAGTCAACCCGTAGATCC | SEQ ID NO: 70 |
| hsa-miR-99a-5p | ATAGACTCCTCGCATAGCCTCATGAGTCTCCCTGAGACC | SEQ ID NO: 71 |
| hsa-miR-125b-5p | ATAGACTCCTCGCATAGCCTCATGAGTCCATTGCACTTGT | SEQ ID NO: 72 |
| hsa-miR-25-3p | ATAGACTCCTCGCATAGCCTCATGAGTCAGCAGCATTGT | SEQ ID NO: 73 |
| hsa-miR-103a-3p | ATAGACTCCTCGCATAGCCTCATGAGTCTCTGGGCAAC | SEQ ID NO: 74 |
| hsa-miR-1285-3p | ATAGACTCCTCGCATAGCCTCATGAGTCTTCCCAGCC | SEQ ID NO: 75 |
| hsa-miR-7977 | ATAGACTCCTCGCATAGCCTCATGAGTCTGTAAACATCCTA | SEQ ID NO: 76 |
| hsa-miR-30b-5p | ATAGACTCCTCGCATAGCCTCATGAGTCAATTGCACGGT | SEQ ID NO: 77 |
| hsa-miR-363-3p | ATAGACTCCTCGCATAGCCTCATGAGTCCAAAGTGCTGT | SEQ ID NO: 78 |
| hsa-miR-93-5p | ATAGACTCCTCGCATAGCCTCATGAGTCTTTGTTCGTTCG | SEQ ID NO: 79 |
| hsa-miR-375-3p | ATAGACTCCTCGCATAGCCTCATGAGTCCACCCGTAGAA | SEQ ID NO: 80 |
| hsa-miR-99b-5p | ATAGACTCCTCGCATAGCCTCATGAGTCAACTGGCCCT | SEQ ID NO: 81 |
| hsa-miR-193b-3p | ATAGACTCCTCGCATAGCCTCATGAGTCACTGCCCCA | SEQ ID NO: 82 |
| hsa-miR-324-3p | ATAGACTCCTCGCATAGCCTCATGAGTCAACTGGCCTAC | SEQ ID NO: 83 |
| hsa-miR-193a-3p | ATAGACTCCTCGCATAGCCTCATGAGTCTCTCACACAG | SEQ ID NO: 84 |
| hsa-miR-342-3p | ATAGACTCCTCGCATAGCCTCATGAGTCTCAGGCTCAGT | SEQ ID NO: 85 |
| hsa-miR-484 | ATAGACTCCTCGCATAGCCTCATGAGTCCCTCCCACAC | SEQ ID NO: 86 |
| hsa-miR-532-3p | ATAGACTCCTCGCATAGCCTCATGAGTCCTGTGCGTGT | SEQ ID NO: 87 |
| hsa-miR-210-3p | ATAGACTCCTCGCATAGCCTCATGAGTCTTGGGGAAACG | SEQ ID NO: 88 |
| hsa-miR-2110 | ATAGACTCCTCGCATAGCCTCATGAGTCAGGGCCCCC | SEQ ID NO: 89 |
| hsa-miR-296-5p | ATAGACTCCTCGCATAGCCTCATGAGTCTCGACCGGAC | SEQ ID NO: 90 |
| hsa-miR-1307-5p | ATAGACTCCTCGCATAGCCTCATGAGTCTGTGCAAATCTA | SEQ ID NO: 91 |
| hsa-miR-19a-3p | ATAGACTCCTCGCATAGCCTCATGAGTCTCTACAGTGCAC | SEQ ID NO: 92 |
| hsa-miR-139-5p | ATAGACTCCTCGCATAGCCTCATGAGTCAGCAGGTGCG | SEQ ID NO: 93 |
| hsa-miR-3665 | ATAGACTCCTCGCATAGCCTCATGAGTCTACCACAGGGTA | SEQ ID NO: 94 |
| hsa-miR-RG-84 | ATAGACTCCTCGCATAGCCTCATGAGTCGGATCCGAGTC | SEQ ID NO: 95 |
| hsa-miR-4454 | ATAGACTCCTCGCATAGCCTCATGAGTCTGAGGTAGTAGG | SEQ ID NO: 96 |
| hsa-let-7b-5p | ATAGACTCCTCGCATAGCCTCATGAGTCAAAAGTGCTTACAG | SEQ ID NO: 97 |

2. A reaction procedure of a PCR instrument was set as follows:

(1) 25° C. for 10 min; (2) 95° C. for 10 min; (3) 3 cycles of (95° C. for 10 s and 55° C. for 10 min); (4) 3 cycles of (95° C. for 10 s and 50° C. for 10 min); (5) 2 cycles of (95° C. for 10 s and 45° C. for 10 min); (6) 2 cycles of (95° C. for 10 s and 40° C. for 10 min); (7) 2 cycles of (95° C. for 10 s and 37° C. for 10 min); (8) 1 cycle of (95° C. for 10 s, 60° C. for 2 min, and 72° C. for 10 min); and (9) a first PCR tube was incubated at 72° C. for 5 min, and then taken out and immediately incubated in an ice box to terminate an activity of the Taq DNA polymerase.

3. After a liquid in the first PCR tube was frozen (about 3 min later), the first PCR tube was placed on a 96-well heat-preservation module (which was frozen to −40° C. in advance).

4. 20 μL of chloroform was added, and then the heat-preservation module was immediately vortexed by a vortex until ice cubes melted (about 1 min later).

5. A resulting system was centrifuged at 12,000 rpm and 4° C. for 15 min, and a part (typically 18 μL) of a resulting supernatant was pipetted by a pipette and added to a labeled second PCR tube.

6. The second PCR tube was carefully centrifuged in a mini centrifuge until the whole sample was precipitated to a bottom of the second PCR tube, then a cap of the second PCR tube was removed, and the second PCR tube with the cap removed was placed in a PCR instrument at 50° C. for 10 min to allow the chloroform completely volatilized.

7. 2.5 μL of an EXO I enzyme was added to the second PCR tube, the second PCR tube was inverted up and down for thorough mixing, then carefully centrifuged, and placed in a PCR instrument with a PCR procedure of 37° C. for 4 min, and 5 s before the end of the procedure, the PCR instrument was paused.

8. The following PCR procedure was set: 37° C. for 4 min and 80° C. for 1 min, and then the PCR instrument was started.

9. The second PCR tube was carefully centrifuged until the whole sample was precipitated to a bottom of the second PCR tube.

### Second PCR Preamplification

1. In a 0.2 mL PCR tube, a first PCR preamplification product solution was inverted up and down several times for thorough mixing and then carefully centrifuged in a mini centrifuge until the whole sample was precipitated to a bottom of the PCR tube.

For a sample, a 20 μL reaction system was prepared specifically from the following reagents: 2× Boost mix: 10 μL, 10 μm magnetic bead-purified transition primer (USEXPnb, TCTACAGATCCTGGCCTCTGACTCCAGGATCTGTAGACCTCCATCCGAGACACACGAT, SEQ ID NO: 99): 1 μL, 10 μm isomiR primer (IsomiRupb, GTTTGTTGCTACGCTCAGAATCCTAAGCGTAGCAACAAACATAGACTCCTCGCATAGCCT CATGAGTC, SEQ ID NO: 100): 1 μL, Tth RecA (0.2 μg/μL): 1 μL, and first PCR preamplification product: 7 μL.

The 2× Boost mix* (including UDG) was prepared with a dNTP mixed solution without dUTP.

2. A Touch Down PCR procedure of the PCR instrument was set as follows:

(1) 25° C. for 10 min; (2) 95° C. for 10 min; (3) 3 cycles of (95° C. for 10 s and 65° C. for 10 min): (4) 3 cycles of (95° C. for 10 s and 62° C. for 10 min): (5) 2 cycles of (95° C. for 10 s and 58° C. for 2 min); (6) 2 cycles of (95° C. for 10 s and 60° C. for 2 min); (7) 1 cycle of (95° C. for 10 s, 60° C. for 2 min, and 72° C. for 10 min); and (8) a first PCR tube was incubated at 72° C. for 5 min, and then taken out and immediately incubated in an ice bath to stop a Taq activity.

3. After a liquid in the first PCR tube was frozen (about 3 min later), the first PCR tube was placed on a 96-well heat-preservation module (which was frozen to −40° C. in advance).

4. 20 μL of chloroform was added, and then the heat-preservation module was immediately vortexed by a vortex until ice cubes melted (about 1 min later).

5. A resulting system was centrifuged at 12,000 rpm and 4° C. for 15 min, and a part (typically 18 μL) of a resulting supernatant was pipetted by a pipette and added to a labeled second PCR tube.

6. The second PCR tube was carefully centrifuged in a mini centrifuge until the whole sample was precipitated to a bottom of the second PCR tube, then a cap of the second PCR tube was removed, and the second PCR tube with the cap removed was placed in a PCR instrument at 50° C. for 10 min to allow the chloroform completely volatilized.

7. 4 μL of washed streptavidin magnetic beads was added to every 20 μL of a reaction solution obtained above (the streptavidin magnetic beads were thoroughly mixed by a vortex and then used immediately).

8. The second PCR tube was shaken on a shaker at a rotational speed of 500 rpm and room temperature for 30 min.

9. The second PCR tube was vortexed by a vortex to make the magnetic beads fully suspended, and then incubated in a PCR instrument at 50° C. for 3 min.

10. The second PCR tube was placed on a magnetic separator for about 1 min to adsorb the magnetic beads, and a resulting solution was pipetted by a pipette (the magnetic beads should not be pipetted as much as possible) and added to a labeled third PCR tube.

### Third PCR Preamplification

1. In a 0.2 mL PCR tube, a second PCR preamplification product solution was inverted up and down several times for thorough mixing and then carefully centrifuged in a mini centrifuge until the whole sample was precipitated to a bottom of the PCR tube.

For a sample, a 20 μL reaction system was prepared specifically from the following reagents: 2× Boost mix*: 10 μL, 10 μm UFP (CAGAATCCTAAGCGTAGCAACAAAC, SEQ ID NO: 101): 1 μL, 10 μm URP (GCCTCTGACTCCAGGATCTGTAGAC, SEQ ID NO: 102): 1 μL, Tth RecA (0.2 μg/μL): 1 μL, and second PCR preamplification product: 7 μL.

The 2× Boost mix* (including UDG) was prepared with a dNTP mixed solution without dUTP.

2. A PCR procedure of a PCR instrument was set as follows:

(1) 95° C. for 10 min; (2) 12 cycles of (95° C. for 10 s and 65° C. for 1 min); (4) 72° C. for 10min; and (5) 72° C. for 5 min, and then a first PCR tube was taken out and immediately immersed in an isopropanol-filled programmed cooling box cryopreserved at −80° C. to terminate an activity of the Tay DNA polymerase (which could avoid non-specific amplification caused by a temperature reduction).

3. After a liquid in the first PCR tube was frozen (about 3 min later), the first PCR tube was placed on a 96-well heat-preservation module (which was frozen to −40° C. in advance).

4. 20 μL of chloroform was added, and then the heat-preservation module was immediately vortexed by a vortex until ice cubes melted (about 1 min later).

5. A resulting system was centrifuged at 12,000 rpm and 4° C. for 15 min, and a part (typically 18 μL) of a resulting supernatant was pipetted by a pipette and added to a labeled second PCR tube.

6. The second PCR tube was carefully centrifuged in a mini centrifuge until the whole sample was precipitated to a bottom of the second PCR tube, then a cap of the second PCR tube was removed, and the second PCR tube with the cap removed was placed in a PCR instrument at 50° C. for 10 min to allow the chloroform completely volatilized.

7. 2.5 μL of an EXO I (Thermolabile) mixed solution was added per reaction (20 μL).

8. The following PCR procedure was set: 37° C. for 4 min and 80° C. for 1 min, and then the PCR instrument was started.

9. 5 μL of a resulting reaction system was taken and 10-fold diluted with 0.1× TE, and then used as a PCR template for subsequent detection.

### II. qPCR Amplification Detection

According to instructions of a manufacturer, a PCR mixture was prepared from the following reagents: 2× DNA polymerase mixture, 0.2 μM (final concentration) each of a forward primer (UFP: CAGAATCCTAAGCGTAGCAACAAAC, SEQ ID NO: 101) and a universal reverse primer (URP: GCCTCTGACTCCAGGATCTGTAGAC, SEQ ID NO: 102), and 0.2 μM (final concentration) LNAFAM probe (ACC+AT+CA+AT+CG+TG+TG (SEQ ID NO: 2064), where + represents an LNA). An amount of a PCR template in a 10 μL PCR system was as follows: 0.08 μL of a third PCR preamplification product 10-fold diluted.

PCR cycling parameters were as follows: 95° C. for 10 min (USQ-miR DNA polymerase mixture) or 1 min (other 2× DNA polymerase mixture), then 95° C. for 30 s, and 65° C. for 1 min, with 40 cycles.

### III. PCR Amplification for Adding a Barcode and an Adapter (a Sequencing Adapter) to a Preamplification Product

### A. PCR Amplification for Adding a Barcode and an Adapter to a Preamplification Product

### 1. Design of Primers for Adding a Barcode and an Adapter

A forward primer was designed as follows:
a sequence overlapping with the forward primer for adding the adapter+IUDI (I5 Index)+a sequence partially overlapping with a 5′-terminus universal sequence of a reverse-transcription product of cDNA.

A reverse primer was designed as follows:

a sequence overlapping with a reverse primer for adding the adapter+IUDI (I7 Index)+a sequence partially overlapping with a 3′-terminus sequence of an isomiR primer.

The I5 Index and the I7 Index are combined in sets, and specific combination modes and specific sequences are shown in Table 2.

TABLE 2
DUDIs for high-throughput samples of NGS

| No. | I5 Index | Sequence No. | I7 Index | Sequence No. |
|---|---|---|---|---|
| 1 | TCAGTATCCT | SEQ ID NO: 103 | CACGCCAACG | SEQ ID NO: 1079 |
| 2 | GCCGAATAGC | SEQ ID NO: 104 | TAAGTAACGA | SEQ ID NO: 1080 |
| 3 | TTACCAGACT | SEQ ID NO: 105 | ACAAGAATCC | SEQ ID NO: 1081 |
| 4 | TTCGCAGCTT | SEQ ID NO: 106 | TAGTTCACCA | SEQ ID NO: 1082 |
| 5 | TCGCAATCTT | SEQ ID NO: 107 | ACACCGACCT | SEQ ID NO: 1083 |
| 6 | TGCCTGATAG | SEQ ID NO: 108 | ACCAATGTAA | SEQ ID NO: 1084 |
| 7 | TGACGACTCT | SEQ ID NO: 109 | ATTCAGTAAG | SEQ ID NO: 1085 |
| 8 | GCATAGACCG | SEQ ID NO: 110 | CCTCGCCTGA | SEQ ID NO: 1086 |
| 9 | TTCCGCGCTT | SEQ ID NO: 111 | ACGAGATAGA | SEQ ID NO: 1087 |
| 10 | GATTGCTGAC | SEQ ID NO: 112 | TGCTCGCCTA | SEQ ID NO: 1088 |
| 11 | GACATAGACG | SEQ ID NO: 113 | CCTATTCGGC | SEQ ID NO: 1089 |
| 12 | GAACCTAATC | SEQ ID NO: 114 | CCGCTGAACC | SEQ ID NO: 1090 |
| 13 | GTAGTAAGAC | SEQ ID NO: 115 | TAGCAGTATC | SEQ ID NO: 1091 |
| 14 | TGCAGTTCTT | SEQ ID NO: 116 | AGACATTACG | SEQ ID NO: 1092 |
| 15 | TTAACATTAC | SEQ ID NO: 117 | CCTTACCTCA | SEQ ID NO: 1093 |
| 16 | GAACTCACGC | SEQ ID NO: 118 | CAGTACGAAT | SEQ ID NO: 1094 |
| 17 | GACGCGCAGA | SEQ ID NO: 119 | TGATAACCTA | SEQ ID NO: 1095 |
| 18 | GGTTCCTTAG | SEQ ID NO: 120 | CCTGATTACG | SEQ ID NO: 1096 |
| 19 | TCCGGCACAC | SEQ ID NO: 121 | CACTGAAGCA | SEQ ID NO: 1097 |
| 20 | GCCTAACTTC | SEQ ID NO: 122 | TGTATTCCAT | SEQ ID NO: 1098 |
| 21 | CAGCACAAGA | SEQ ID NO: 123 | CCTCAAGCCA | SEQ ID NO: 1099 |
| 22 | TAGCAGCTCA | SEQ ID NO: 124 | TAGCAAGCCA | SEQ ID NO: 1100 |
| 23 | ACGCGCCAGA | SEQ ID NO: 125 | ACTTGCCACG | SEQ ID NO: 1101 |
| 24 | ACTCTTGGTT | SEQ ID NO: 126 | CAGACGCCGG | SEQ ID NO: 1102 |
| 25 | TTAATCTTCA | SEQ ID NO: 127 | CAACTAATCG | SEQ ID NO: 1103 |
| 26 | TCATTATTAT | SEQ ID NO: 128 | TGGACTCGCA | SEQ ID NO: 1104 |
| 27 | GCTCACGCAC | SEQ ID NO: 129 | CGCCGACAAC | SEQ ID NO: 1105 |
| 28 | TGTGACTGTG | SEQ ID NO: 130 | CCAGATAATG | SEQ ID NO: 1106 |
| 29 | TTAACTCTCG | SEQ ID NO: 131 | TGAGATAGTA | SEQ ID NO: 1107 |
| 30 | TTACGGCGCA | SEQ ID NO: 132 | AACTGACGAG | SEQ ID NO: 1108 |
| 31 | TTCTCGCCAC | SEQ ID NO: 133 | TAAGCCGATG | SEQ ID NO: 1109 |
| 32 | GGCTCCTACG | SEQ ID NO: 134 | CATTGACACT | SEQ ID NO: 1110 |
| 33 | GACTGCCGCG | SEQ ID NO: 135 | CCTTGATAAT | SEQ ID NO: 1111 |
| 34 | GACAGTTCTC | SEQ ID NO: 136 | TAGTATGACG | SEQ ID NO: 1112 |
| 35 | TGTCCATCAT | SEQ ID NO: 137 | AGAACTGCTC | SEQ ID NO: 1113 |
| 36 | GACCGCTAAG | SEQ ID NO: 138 | TACAATTCCA | SEQ ID NO: 1114 |
| 37 | GCTCGAATAA | SEQ ID NO: 139 | TGTACCTAGA | SEQ ID NO: 1115 |
| 38 | TGGTCAGTCG | SEQ ID NO: 140 | TAATCCATTC | SEQ ID NO: 1116 |
| 39 | GGTTACTCTG | SEQ ID NO: 141 | TGCCTCCATG | SEQ ID NO: 1117 |
| 40 | CAACAGTTCG | SEQ ID NO: 142 | ATACCACGGC | SEQ ID NO: 1118 |
| 41 | TGGCAGTGGT | SEQ ID NO: 143 | AGTTGTATTC | SEQ ID NO: 1119 |
| 42 | TGTTCTGACG | SEQ ID NO: 144 | TAGCTCCATT | SEQ ID NO: 1120 |
| 43 | CAACACGATC | SEQ ID NO: 145 | ATTGCAGTAA | SEQ ID NO: 1121 |
| 44 | CATCAATCAT | SEQ ID NO: 146 | TGTTCAATAG | SEQ ID NO: 1122 |
| 45 | GCACTCCTTA | SEQ ID NO: 147 | CCGGTGACGG | SEQ ID NO: 1123 |
| 46 | AGCATCCAGA | SEQ ID NO: 148 | CGGTATCATA | SEQ ID NO: 1124 |
| 47 | CACTGCATAC | SEQ ID NO: 149 | AACTACTACG | SEQ ID NO: 1125 |
| 48 | GGTGCAGACG | SEQ ID NO: 150 | CCAATTACTG | SEQ ID NO: 1126 |
| 49 | CGCAACGCCG | SEQ ID NO: 151 | CCGCACGCTA | SEQ ID NO: 1127 |
| 50 | AAGACTCTGA | SEQ ID NO: 152 | CCTTGGTATG | SEQ ID NO: 1128 |
| 51 | TGCCTCTAAT | SEQ ID NO: 153 | TGCAGCACGA | SEQ ID NO: 1129 |
| 52 | CGCAGTACGC | SEQ ID NO: 154 | ATAGCCAAGC | SEQ ID NO: 1130 |
| 53 | CATTGCTTGG | SEQ ID NO: 155 | TTAGTAGACC | SEQ ID NO: 1131 |
| 54 | GTAAGATATT | SEQ ID NO: 156 | TAAGAACTAA | SEQ ID NO: 1132 |
| 55 | GGAACAGACT | SEQ ID NO: 157 | CACGATTAAG | SEQ ID NO: 1133 |
| 56 | GTAAGACCGG | SEQ ID NO: 158 | CACAGTGTAG | SEQ ID NO: 1134 |
| 57 | TGCCTAAGTC | SEQ ID NO: 159 | ACACGAATTG | SEQ ID NO: 1135 |
| 58 | TAGACATATT | SEQ ID NO: 160 | TAGCACCGAC | SEQ ID NO: 1136 |
| 59 | GACTTATCCT | SEQ ID NO: 161 | CAAGAATAAC | SEQ ID NO: 1137 |
| 60 | TCGCATCGAA | SEQ ID NO: 162 | AAGCCGCACT | SEQ ID NO: 1138 |
| 61 | ACTTAGTTAC | SEQ ID NO: 163 | AGTTCAGATT | SEQ ID NO: 1139 |
| 62 | TCACAGTCAC | SEQ ID NO: 164 | TCACCACGAT | SEQ ID NO: 1140 |
| 63 | GGCCTCTTGG | SEQ ID NO: 165 | CAGCGATTGT | SEQ ID NO: 1141 |
| 64 | GTAGACCAAT | SEQ ID NO: 166 | TGCCAGCGCG | SEQ ID NO: 1142 |
| 65 | GTAATATCAG | SEQ ID NO: 167 | TGGCTCCTCA | SEQ ID NO: 1143 |
| 66 | AATTCGATGC | SEQ ID NO: 168 | TGACCTCGCC | SEQ ID NO: 1144 |
| 67 | GCTGCGCTAC | SEQ ID NO: 169 | TACGACTCAA | SEQ ID NO: 1145 |
| 68 | GATGTCCTTC | SEQ ID NO: 170 | TAATTGCCAA | SEQ ID NO: 1146 |
| 69 | AACTCTTGTG | SEQ ID NO: 171 | AACGGCGATA | SEQ ID NO: 1147 |
| 70 | GCGCCGCGCT | SEQ ID NO: 172 | CTCGATTCCA | SEQ ID NO: 1148 |
| 71 | TAGACTACTC | SEQ ID NO: 173 | ATACGCTTCG | SEQ ID NO: 1149 |
| 72 | TCCTGACACA | SEQ ID NO: 174 | TGATGATGAT | SEQ ID NO: 1150 |
| 73 | GAATACCAAG | SEQ ID NO: 175 | CTACCTGAAT | SEQ ID NO: 1151 |
| 74 | GCCTGCCGAC | SEQ ID NO: 176 | CTACACTCAA | SEQ ID NO: 1152 |
| 75 | TGGCCGATAC | SEQ ID NO: 177 | TTGTGATAGC | SEQ ID NO: 1153 |
| 76 | TCCGACGTAT | SEQ ID NO: 178 | CAATTCGCGC | SEQ ID NO: 1154 |
| 77 | ACAGTTACTA | SEQ ID NO: 179 | CATGGCATTG | SEQ ID NO: 1155 |
| 78 | GCACCTAGAC | SEQ ID NO: 180 | CTTCTGACTT | SEQ ID NO: 1156 |
| 79 | ACTACGTCCT | SEQ ID NO: 181 | AGAGAACCAA | SEQ ID NO: 1157 |
| 80 | CTCATTATTC | SEQ ID NO: 182 | ATTCACAAGA | SEQ ID NO: 1158 |
| 81 | TGACACAACT | SEQ ID NO: 183 | CACCAGCTAA | SEQ ID NO: 1159 |
| 82 | GAGAATAGCT | SEQ ID NO: 184 | CAAGTTAGCG | SEQ ID NO: 1160 |
| 83 | GATGCCTCAA | SEQ ID NO: 185 | TGCGCCTTCG | SEQ ID NO: 1161 |
| 84 | GAGACACTGC | SEQ ID NO: 186 | CCAACCACAT | SEQ ID NO: 1162 |
| 85 | ACACTGCTCT | SEQ ID NO: 187 | ACGCCATGTA | SEQ ID NO: 1163 |
| 86 | GAATGTTACC | SEQ ID NO: 188 | CAGTTAGACG | SEQ ID NO: 1164 |
| 87 | GCGCGAAGCC | SEQ ID NO: 189 | CCTCAATTAG | SEQ ID NO: 1165 |
| 88 | TGTGCGCCGA | SEQ ID NO: 190 | AACACTGGTA | SEQ ID NO: 1166 |
| 89 | AGCTGCACTG | SEQ ID NO: 191 | AATCCGCTAA | SEQ ID NO: 1167 |
| 90 | GACCTAATCT | SEQ ID NO: 192 | TGGAACCATA | SEQ ID NO: 1168 |
| 91 | TCTAGCTGCT | SEQ ID NO: 193 | TCGCTCAACA | SEQ ID NO: 1169 |
| 92 | TTGCCACGCG | SEQ ID NO: 194 | ACTACCAGTA | SEQ ID NO: 1170 |
| 93 | GGATTAGCGA | SEQ ID NO: 195 | CCTGAACCGA | SEQ ID NO: 1171 |
| 94 | GCGCTCTCAT | SEQ ID NO: 196 | TTGCCGACTC | SEQ ID NO: 1172 |
| 95 | GAGCTACTCC | SEQ ID NO: 197 | CCTAGACGCT | SEQ ID NO: 1173 |
| 96 | TCGCACTGGC | SEQ ID NO: 198 | ATACCAACTA | SEQ ID NO: 1174 |
| 97 | GAAGTTCTCT | SEQ ID NO: 199 | CAATACCACC | SEQ ID NO: 1175 |
| 98 | ACATTAAGTG | SEQ ID NO: 200 | ATGAGAGAAC | SEQ ID NO: 1176 |
| 99 | GCTCCTCAGA | SEQ ID NO: 201 | GGAACTAAGT | SEQ ID NO: 1177 |
| 100 | CAGATGTACG | SEQ ID NO: 202 | AGATAGAACC | SEQ ID NO: 1178 |
| 101 | ATCCTCAGCT | SEQ ID NO: 203 | AATTACTCCA | SEQ ID NO: 1179 |
| 102 | CTCTGCCAAC | SEQ ID NO: 204 | TGCTTCAATT | SEQ ID NO: 1180 |
| 103 | GTGGCAAGCC | SEQ ID NO: 205 | CAGTGGTACA | SEQ ID NO: 1181 |
| 104 | GAAGTTGACG | SEQ ID NO: 206 | GGCGGTTGTG | SEQ ID NO: 1182 |
| 105 | ACTCGTTCCG | SEQ ID NO: 207 | ACACGGTGCC | SEQ ID NO: 1183 |
| 106 | GGCTTGGTCG | SEQ ID NO: 208 | CATGTCACTA | SEQ ID NO: 1184 |
| 107 | AGCCTTCTAG | SEQ ID NO: 209 | CTACTGATGT | SEQ ID NO: 1185 |
| 108 | TCTACTGCTT | SEQ ID NO: 210 | ACAGCCTTAC | SEQ ID NO: 1186 |
| 109 | ACCTCAATAC | SEQ ID NO: 211 | AGTTACAGCG | SEQ ID NO: 1187 |
| 110 | GCTCTCAACT | SEQ ID NO: 212 | TATATAGATT | SEQ ID NO: 1188 |
| 111 | TCTCTTCAAG | SEQ ID NO: 213 | TACCGCAATC | SEQ ID NO: 1189 |
| 112 | TCGGACGGTG | SEQ ID NO: 214 | ACGATAGTGG | SEQ ID NO: 1190 |
| 113 | CGCTCTCCAA | SEQ ID NO: 215 | ATACGATAGC | SEQ ID NO: 1191 |
| 114 | GTAAGCGGTT | SEQ ID NO: 216 | CGGTTCCTCG | SEQ ID NO: 1192 |
| 115 | GCATTGAAGC | SEQ ID NO: 217 | TACGGAGTAA | SEQ ID NO: 1193 |
| 116 | ATATCAAGCA | SEQ ID NO: 218 | ACACGCATAA | SEQ ID NO: 1194 |
| 117 | ATCCTAGCGC | SEQ ID NO: 219 | CATAAGATTC | SEQ ID NO: 1195 |
| 118 | TAGTTGTTGT | SEQ ID NO: 220 | TACCAGACGC | SEQ ID NO: 1196 |
| 119 | TCGTCCTACG | SEQ ID NO: 221 | TCATTCCTAA | SEQ ID NO: 1197 |
| 120 | CGAACGATCT | SEQ ID NO: 222 | CATACGAATA | SEQ ID NO: 1198 |
| 121 | TTACAACACA | SEQ ID NO: 223 | CTTGAACACT | SEQ ID NO: 1199 |
| 122 | TCTGACGACA | SEQ ID NO: 224 | TGACGGCTAA | SEQ ID NO: 1200 |
| 123 | TCTGAATCTG | SEQ ID NO: 225 | TATGTAAGCT | SEQ ID NO: 1201 |
| 124 | TTATTGAATA | SEQ ID NO: 226 | ACGGACCAGC | SEQ ID NO: 1202 |
| 125 | AGGACCACGC | SEQ ID NO: 227 | GGCAGATGAG | SEQ ID NO: 1203 |
| 126 | CTCCACCGAT | SEQ ID NO: 228 | AGAAGTATAG | SEQ ID NO: 1204 |
| 127 | GATGGTGACC | SEQ ID NO: 229 | TAATAATCTG | SEQ ID NO: 1205 |
| 128 | TTAGTGTCAA | SEQ ID NO: 230 | AATCGCCTCG | SEQ ID NO: 1206 |
| 129 | GTTCTTCATG | SEQ ID NO: 231 | CCTTGTGGTG | SEQ ID NO: 1207 |
| 130 | TCAGGTGATC | SEQ ID NO: 232 | GGAATAGATA | SEQ ID NO: 1208 |
| 131 | CTCTCATTGA | SEQ ID NO: 233 | CAGAAGTTGG | SEQ ID NO: 1209 |
| 132 | GCAAGTGGTC | SEQ ID NO: 234 | TGGTAGAGTT | SEQ ID NO: 1210 |
| 133 | ACCAGTACTT | SEQ ID NO: 235 | ATTCACCAAT | SEQ ID NO: 1211 |
| 134 | GTGCTAATCG | SEQ ID NO: 236 | CCTGGTAACT | SEQ ID NO: 1212 |
| 135 | TCACGTACTC | SEQ ID NO: 237 | CATCGGTAGA | SEQ ID NO: 1213 |
| 136 | AGCCGCGCAC | SEQ ID NO: 238 | TTCTTGACTT | SEQ ID NO: 1214 |
| 137 | GCAACAATTA | SEQ ID NO: 239 | CTGACCACCA | SEQ ID NO: 1215 |
| 138 | GAATCGACGG | SEQ ID NO: 240 | TGAGCGGCGG | SEQ ID NO: 1216 |
| 139 | ATCTAGCTCT | SEQ ID NO: 241 | ACGGAGACAG | SEQ ID NO: 1217 |
| 140 | AACTGAACGT | SEQ ID NO: 242 | CATATAACAG | SEQ ID NO: 1218 |
| 141 | GGAGCAGCAC | SEQ ID NO: 243 | CACTCACACC | SEQ ID NO: 1219 |
| 142 | GCGGAACGCC | SEQ ID NO: 244 | CCTGCCTCAC | SEQ ID NO: 1220 |
| 143 | GTTACATGCC | SEQ ID NO: 245 | TGAAGTTGAG | SEQ ID NO: 1221 |
| 144 | GTTGGCAGAC | SEQ ID NO: 246 | CATAGCGACC | SEQ ID NO: 1222 |
| 145 | AGTTATTGTT | SEQ ID NO: 247 | ACAGCGACGC | SEQ ID NO: 1223 |
| 146 | TCGATGCTTA | SEQ ID NO: 248 | ACTGCTCGCT | SEQ ID NO: 1224 |
| 147 | GTTGCTCTAA | SEQ ID NO: 249 | TTCATTGGCG | SEQ ID NO: 1225 |
| 148 | GACAGAAGAC | SEQ ID NO: 250 | CATGTATAGT | SEQ ID NO: 1226 |
| 149 | TCTCTGCCAT | SEQ ID NO: 251 | CTACAATAAT | SEQ ID NO: 1227 |
| 150 | GATTCGTTCC | SEQ ID NO: 252 | CACGCATGTT | SEQ ID NO: 1228 |
| 151 | GTAATGAACT | SEQ ID NO: 253 | TGGAGCCACG | SEQ ID NO: 1229 |
| 152 | AGACATACCA | SEQ ID NO: 254 | AATGTGACGG | SEQ ID NO: 1230 |
| 153 | ATCAACTGAG | SEQ ID NO: 255 | AACTGGCACA | SEQ ID NO: 1231 |
| 154 | CTGGACTCGA | SEQ ID NO: 256 | TGAGACGCGC | SEQ ID NO: 1232 |
| 155 | GAACTAGAGC | SEQ ID NO: 257 | CAGTGAGCAT | SEQ ID NO: 1233 |
| 156 | GATCAACAGC | SEQ ID NO: 258 | CGCATATTCC | SEQ ID NO: 1234 |
| 157 | GCGTAGCCGA | SEQ ID NO: 259 | CTAGAGATAG | SEQ ID NO: 1235 |
| 158 | TGCACAATGG | SEQ ID NO: 260 | AACCTCTACC | SEQ ID NO: 1236 |
| 159 | GGTATCTTGC | SEQ ID NO: 261 | CTCATGTTAA | SEQ ID NO: 1237 |
| 160 | TCTAACTGTA | SEQ ID NO: 262 | ACGCAATTCA | SEQ ID NO: 1238 |
| 161 | CGCGCTACTT | SEQ ID NO: 263 | CACTCCATCA | SEQ ID NO: 1239 |
| 162 | GTTAATGAGC | SEQ ID NO: 264 | TACCGCTGAT | SEQ ID NO: 1240 |
| 163 | AACACAATGC | SEQ ID NO: 265 | AGTTGACCAT | SEQ ID NO: 1241 |
| 164 | GCCGGTCGCG | SEQ ID NO: 266 | CTTACTGCCA | SEQ ID NO: 1242 |
| 165 | TAGAAGTGCT | SEQ ID NO: 267 | ATATGGTAGA | SEQ ID NO: 1243 |
| 166 | AGTAGCGCGG | SEQ ID NO: 268 | AGATCACGAG | SEQ ID NO: 1244 |
| 167 | TGCACGTTCA | SEQ ID NO: 269 | TGTAGCGGCC | SEQ ID NO: 1245 |
| 168 | TAGCAACTAT | SEQ ID NO: 270 | CCGCCACTCT | SEQ ID NO: 1246 |
| 169 | GACCGCGTTC | SEQ ID NO: 271 | CGACCTTACC | SEQ ID NO: 1247 |
| 170 | GAGTGACGAT | SEQ ID NO: 272 | CATCGAGAGT | SEQ ID NO: 1248 |
| 171 | GCTACTACTG | SEQ ID NO: 273 | CCTGGTATGG | SEQ ID NO: 1249 |
| 172 | AAGCAAGGTC | SEQ ID NO: 274 | ACGGTCAGAA | SEQ ID NO: 1250 |
| 173 | TGTCTTCGGT | SEQ ID NO: 275 | CACTTGTATA | SEQ ID NO: 1251 |
| 174 | CGCGCTAACC | SEQ ID NO: 276 | CTTCGACCTC | SEQ ID NO: 1252 |
| 175 | CAGTTCTGAA | SEQ ID NO: 277 | TTAGTGCATT | SEQ ID NO: 1253 |
| 176 | ACGTTACTAG | SEQ ID NO: 278 | AGAGTTAAGC | SEQ ID NO: 1254 |
| 177 | GAGACGGAAT | SEQ ID NO: 279 | CAGCGGAGCA | SEQ ID NO: 1255 |
| 178 | TAGCTTGCGC | SEQ ID NO: 280 | CGATTACCTC | SEQ ID NO: 1256 |
| 179 | GCAAGTGACA | SEQ ID NO: 281 | CGACCATCCT | SEQ ID NO: 1257 |
| 180 | TCGCAGGTAT | SEQ ID NO: 282 | GACTATTAGA | SEQ ID NO: 1258 |
| 181 | CTTGCACGAA | SEQ ID NO: 283 | ATAACTGATA | SEQ ID NO: 1259 |
| 182 | AGTGGAACTA | SEQ ID NO: 284 | ATGAATCAGC | SEQ ID NO: 1260 |
| 183 | GGATAACTAT | SEQ ID NO: 285 | TACCTTGTTC | SEQ ID NO: 1261 |
| 184 | GCCTGGTGTG | SEQ ID NO: 286 | TTAGATGCTG | SEQ ID NO: 1262 |
| 185 | ATCGCTCCAA | SEQ ID NO: 287 | CATACCGCTT | SEQ ID NO: 1263 |
| 186 | GTTGCTGTGC | SEQ ID NO: 288 | TGTTGCGGTG | SEQ ID NO: 1264 |
| 187 | TTAAGTGCGC | SEQ ID NO: 289 | AGATCCTGAT | SEQ ID NO: 1265 |
| 188 | GTAGCTGGAC | SEQ ID NO: 290 | TTCCGCTAGA | SEQ ID NO: 1266 |
| 189 | GCTCCACGTT | SEQ ID NO: 291 | TTCAACACAC | SEQ ID NO: 1267 |
| 190 | GATGCTCATT | SEQ ID NO: 292 | TGTATGCACG | SEQ ID NO: 1268 |
| 191 | TCAGCGGCTA | SEQ ID NO: 293 | CTTCAGAACT | SEQ ID NO: 1269 |
| 192 | TTGCCTCGTC | SEQ ID NO: 294 | AAGACCACTG | SEQ ID NO: 1270 |
| 193 | ACCTCCGAAC | SEQ ID NO: 295 | TGCAGATTGT | SEQ ID NO: 1271 |
| 194 | CGATCCATAT | SEQ ID NO: 296 | AGAACACTGT | SEQ ID NO: 1272 |
| 195 | TCCTCGATCG | SEQ ID NO: 297 | CGCACACCAG | SEQ ID NO: 1273 |
| 196 | GGCGGACACA | SEQ ID NO: 298 | CGCATAGACT | SEQ ID NO: 1274 |
| 197 | GGCTCCGCTA | SEQ ID NO: 299 | CACTCTACTA | SEQ ID NO: 1275 |
| 198 | AGTGGTAGCG | SEQ ID NO: 300 | TAATCGGTGA | SEQ ID NO: 1276 |
| 199 | GGCTCACGTT | SEQ ID NO: 301 | CTAAGATGCT | SEQ ID NO: 1277 |
| 200 | GGATCTTGCT | SEQ ID NO: 302 | TTCTTGGCCG | SEQ ID NO: 1278 |
| 201 | AACACCTGGT | SEQ ID NO: 303 | CGGTCGAGAC | SEQ ID NO: 1279 |
| 202 | GAGCTGTAAG | SEQ ID NO: 304 | GGACCGAGTG | SEQ ID NO: 1280 |
| 203 | GTATGTGCAG | SEQ ID NO: 305 | CCGCCTCCAA | SEQ ID NO: 1281 |
| 204 | CATCGCTATT | SEQ ID NO: 306 | TGGTATTCAA | SEQ ID NO: 1282 |
| 205 | AGTACTTCAT | SEQ ID NO: 307 | AATAACACCT | SEQ ID NO: 1283 |
| 206 | ACTCGCGGAA | SEQ ID NO: 308 | CTCAGACCTG | SEQ ID NO: 1284 |
| 207 | GGCCGTATGA | SEQ ID NO: 309 | CTAACAGCAC | SEQ ID NO: 1285 |
| 208 | TCCGTCGCCT | SEQ ID NO: 310 | CCAGCAACGT | SEQ ID NO: 1286 |
| 209 | GCTCGGTACT | SEQ ID NO: 311 | CGGACATTGG | SEQ ID NO: 1287 |
| 210 | GCCTGTTATC | SEQ ID NO: 312 | TAAGCTATTG | SEQ ID NO: 1288 |
| 211 | ACTGTACTAC | SEQ ID NO: 313 | AGTGATTCTC | SEQ ID NO: 1289 |
| 212 | ATCTCAGAAT | SEQ ID NO: 314 | ACAGCCGATC | SEQ ID NO: 1290 |
| 213 | CTCCTACTAG | SEQ ID NO: 315 | AGCCAATGAG | SEQ ID NO: 1291 |
| 214 | GGAAGCAGCA | SEQ ID NO: 316 | TATTACCTGG | SEQ ID NO: 1292 |
| 215 | GGCATGTGGA | SEQ ID NO: 317 | TACAATGTGG | SEQ ID NO: 1293 |
| 216 | AGCGATCCGA | SEQ ID NO: 318 | CAACGGAATT | SEQ ID NO: 1294 |
| 217 | GCCACTACAA | SEQ ID NO: 319 | GATGAATGCC | SEQ ID NO: 1295 |
| 218 | AACCGTGCCT | SEQ ID NO: 320 | CCATCACCTA | SEQ ID NO: 1296 |
| 219 | CATCACGGAT | SEQ ID NO: 321 | CACAACTCAT | SEQ ID NO: 1297 |
| 220 | GTCGATTGGT | SEQ ID NO: 322 | CGCCTAACCT | SEQ ID NO: 1298 |
| 221 | GTCAATGTCC | SEQ ID NO: 323 | CACTGCGCTC | SEQ ID NO: 1299 |
| 222 | ATATCCGCCG | SEQ ID NO: 324 | ATCAGACTGG | SEQ ID NO: 1300 |
| 223 | TTAATACAAG | SEQ ID NO: 325 | TATGCAAGTG | SEQ ID NO: 1301 |
| 224 | CTCTGATCTT | SEQ ID NO: 326 | CATACTCTAA | SEQ ID NO: 1302 |
| 225 | AGCCTGGAAC | SEQ ID NO: 327 | AGTGCTTACA | SEQ ID NO: 1303 |
| 226 | GAAGCCTCGG | SEQ ID NO: 328 | CACATACTAA | SEQ ID NO: 1304 |
| 227 | TGGTCGCGCT | SEQ ID NO: 329 | CCACATGGTA | SEQ ID NO: 1305 |
| 228 | GTTAATTCTT | SEQ ID NO: 330 | CGGCTTGTGG | SEQ ID NO: 1306 |
| 229 | GATCTACGCG | SEQ ID NO: 331 | CGAGACTGCA | SEQ ID NO: 1307 |
| 230 | GCAACTGAAT | SEQ ID NO: 332 | GGTGTCCAAT | SEQ ID NO: 1308 |
| 231 | GCCAGCTTGA | SEQ ID NO: 333 | GGTACTCTTG | SEQ ID NO: 1309 |
| 232 | TGTGCATGCT | SEQ ID NO: 334 | AACCATTCAT | SEQ ID NO: 1310 |
| 233 | CAGGTGATCT | SEQ ID NO: 335 | GGAACGCAAG | SEQ ID NO: 1311 |
| 234 | ACGCCTCTTA | SEQ ID NO: 336 | ATGTACTTCC | SEQ ID NO: 1312 |
| 235 | AATCAGCTGC | SEQ ID NO: 337 | TACAACGATC | SEQ ID NO: 1313 |
| 236 | AGACACCTCT | SEQ ID NO: 338 | TACGCATGGC | SEQ ID NO: 1314 |
| 237 | GGTCCTGTCA | SEQ ID NO: 339 | TGGTCCGATA | SEQ ID NO: 1315 |
| 238 | GTAACTGCGA | SEQ ID NO: 340 | CTCGGCGACA | SEQ ID NO: 1316 |
| 239 | TCCGCGTTCT | SEQ ID NO: 341 | TCAATGCTCG | SEQ ID NO: 1317 |
| 240 | TCTCATGGCC | SEQ ID NO: 342 | GATTCAGAGT | SEQ ID NO: 1318 |
| 241 | TCGCGGCTGG | SEQ ID NO: 343 | TCATGGTTGA | SEQ ID NO: 1319 |
| 242 | AAGTTCATAC | SEQ ID NO: 344 | CCTCTCAAGG | SEQ ID NO: 1320 |
| 243 | TCCTAGTCGA | SEQ ID NO: 345 | AATGCAGCCA | SEQ ID NO: 1321 |
| 244 | AATATTGCCA | SEQ ID NO: 346 | TTGAGTGATA | SEQ ID NO: 1322 |
| 245 | CATGGCTGCA | SEQ ID NO: 347 | ACTACCGGCG | SEQ ID NO: 1323 |
| 246 | ATCCTGATTA | SEQ ID NO: 348 | ACATCCTGCC | SEQ ID NO: 1324 |
| 247 | GTGTAACCGG | SEQ ID NO: 349 | CTCTGCAACG | SEQ ID NO: 1325 |
| 248 | GCCTAGCGGT | SEQ ID NO: 350 | TTACAAGCTA | SEQ ID NO: 1326 |
| 249 | TGTGGATAAC | SEQ ID NO: 351 | ACTGCTCTTG | SEQ ID NO: 1327 |
| 250 | GTGACTATTC | SEQ ID NO: 352 | CACGCAGCTG | SEQ ID NO: 1328 |
| 251 | AGCACTCTCG | SEQ ID NO: 353 | AATTGGAGCC | SEQ ID NO: 1329 |
| 252 | AGCTGAACAC | SEQ ID NO: 354 | GCAGATAACA | SEQ ID NO: 1330 |
| 253 | TCTTACCAGA | SEQ ID NO: 355 | CTCATCGATA | SEQ ID NO: 1331 |
| 254 | TCTAATCCTG | SEQ ID NO: 356 | ATCATGACTG | SEQ ID NO: 1332 |
| 255 | GAAGTATTCC | SEQ ID NO: 357 | GAAGTATGAA | SEQ ID NO: 1333 |
| 256 | CAGCTACACT | SEQ ID NO: 358 | ATGTAAGAAG | SEQ ID NO: 1334 |
| 257 | CGTAAGCATT | SEQ ID NO: 359 | ACTAGACGTA | SEQ ID NO: 1335 |
| 258 | TCACTATACG | SEQ ID NO: 360 | AGCTGCCTAG | SEQ ID NO: 1336 |
| 259 | AAGGTATTCG | SEQ ID NO: 361 | GTGCAGCCTA | SEQ ID NO: 1337 |
| 260 | GTTGATACCT | SEQ ID NO: 362 | CTCTGTAAGT | SEQ ID NO: 1338 |
| 261 | ACTGTTCTGA | SEQ ID NO: 363 | CACGACTGGT | SEQ ID NO: 1339 |
| 262 | GTAGACATGC | SEQ ID NO: 364 | TCGAACATCA | SEQ ID NO: 1340 |
| 263 | TCGACCGTAG | SEQ ID NO: 365 | CAGCGTACAA | SEQ ID NO: 1341 |
| 264 | AGAGTAAGTC | SEQ ID NO: 366 | CATAATAATG | SEQ ID NO: 1342 |
| 265 | TTCAAGTCTC | SEQ ID NO: 367 | ACGATAATTC | SEQ ID NO: 1343 |
| 266 | AGACGCTGTG | SEQ ID NO: 368 | AGACTGTGAG | SEQ ID NO: 1344 |
| 267 | GCAGCACGAG | SEQ ID NO: 369 | TTGTTGACGG | SEQ ID NO: 1345 |
| 268 | CATTATGCCT | SEQ ID NO: 370 | ACTCATGTGG | SEQ ID NO: 1346 |
| 269 | GCCACATCAC | SEQ ID NO: 371 | CGGCCATTCA | SEQ ID NO: 1347 |
| 270 | AGTTCCGGAC | SEQ ID NO: 372 | ACCTCATTCT | SEQ ID NO: 1348 |
| 271 | CTCTGTAGTC | SEQ ID NO: 373 | TCCGCACAGC | SEQ ID NO: 1349 |
| 272 | GTCCTCCTAC | SEQ ID NO: 374 | TCGCCGGAGC | SEQ ID NO: 1350 |
| 273 | TCGACAGGTG | SEQ ID NO: 375 | GAGACAACCG | SEQ ID NO: 1351 |
| 274 | GCTTCAGCGC | SEQ ID NO: 376 | GGTACCTTCA | SEQ ID NO: 1352 |
| 275 | ATTGCGGCGG | SEQ ID NO: 377 | AACGCGATAA | SEQ ID NO: 1353 |
| 276 | TCACACTAGT | SEQ ID NO: 378 | CATTCAACAT | SEQ ID NO: 1354 |
| 277 | GCTGGATGCA | SEQ ID NO: 379 | TGAGTGTATA | SEQ ID NO: 1355 |
| 278 | TGTATGTGAG | SEQ ID NO: 380 | CGCAGTGAGA | SEQ ID NO: 1356 |
| 279 | CGTTCCAACC | SEQ ID NO: 381 | ATATGACGCG | SEQ ID NO: 1357 |
| 280 | GCGCTTAGAT | SEQ ID NO: 382 | CACTGCTACC | SEQ ID NO: 1358 |
| 281 | AATCGGTTGG | SEQ ID NO: 383 | AGCTTCAGAC | SEQ ID NO: 1359 |
| 282 | TTCCAAGGAT | SEQ ID NO: 384 | GGCTTAGCAG | SEQ ID NO: 1360 |
| 283 | AGCCGAAGCG | SEQ ID NO: 385 | CGATCAGAGT | SEQ ID NO: 1361 |
| 284 | GTGCATCACC | SEQ ID NO: 386 | CTATCGCTCC | SEQ ID NO: 1362 |
| 285 | CGTCCGGCCT | SEQ ID NO: 387 | ACTTCCGCAG | SEQ ID NO: 1363 |
| 286 | GATATCTAGT | SEQ ID NO: 388 | CAACGGTAAC | SEQ ID NO: 1364 |
| 287 | TGAGCGGACA | SEQ ID NO: 389 | ACCACTTACC | SEQ ID NO: 1365 |
| 288 | CTGTTAGCGA | SEQ ID NO: 390 | CACAGTATCC | SEQ ID NO: 1366 |
| 289 | CCAGACAGTC | SEQ ID NO: 391 | TAACGGTCGC | SEQ ID NO: 1367 |
| 290 | CGTAGATTCA | SEQ ID NO: 392 | TTGCTCACAG | SEQ ID NO: 1368 |
| 291 | AGCTAATCGA | SEQ ID NO: 393 | ACCTACGCGA | SEQ ID NO: 1369 |
| 292 | TTCTCGTCCT | SEQ ID NO: 394 | TCCAATTAGT | SEQ ID NO: 1370 |
| 293 | TGTACTAGTT | SEQ ID NO: 395 | AGAATTATTC | SEQ ID NO: 1371 |
| 294 | GACTTACTGT | SEQ ID NO: 396 | CGAACTGCCG | SEQ ID NO: 1372 |
| 295 | GATAGATATC | SEQ ID NO: 397 | TTGTTGCGCC | SEQ ID NO: 1373 |
| 296 | ACTGATATCC | SEQ ID NO: 398 | ATGCAACCTT | SEQ ID NO: 1374 |
| 297 | GCGAATCTAA | SEQ ID NO: 399 | CCTATCGGTA | SEQ ID NO: 1375 |
| 298 | CTCTCAAGTG | SEQ ID NO: 400 | AATAGTCGAT | SEQ ID NO: 1376 |
| 299 | GGCTCTTCGC | SEQ ID NO: 401 | GAGCAATGAT | SEQ ID NO: 1377 |
| 300 | ACGTCGATCC | SEQ ID NO: 402 | ACAGCTCAGA | SEQ ID NO: 1378 |
| 301 | AATTAAGAAT | SEQ ID NO: 403 | CCACGCTTGT | SEQ ID NO: 1379 |
| 302 | AGCGCTCGAT | SEQ ID NO: 404 | AAGCCTGGCA | SEQ ID NO: 1380 |
| 303 | GTTAGGAACC | SEQ ID NO: 405 | CAGGACAGTG | SEQ ID NO: 1381 |
| 304 | CATGTCGAAC | SEQ ID NO: 406 | TCAAGTTATT | SEQ ID NO: 1382 |
| 305 | GTTCATACAG | SEQ ID NO: 407 | CCACACAATT | SEQ ID NO: 1383 |
| 306 | AACGCCGGCA | SEQ ID NO: 408 | AGAAGATTGC | SEQ ID NO: 1384 |
| 307 | TGTAGAGTCG | SEQ ID NO: 409 | AAGACGGTCC | SEQ ID NO: 1385 |
| 308 | TGCGACCACG | SEQ ID NO: 410 | ACTGAGTGCT | SEQ ID NO: 1386 |
| 309 | TCCTCTCTAT | SEQ ID NO: 411 | AGACGCGAGA | SEQ ID NO: 1387 |
| 310 | GTAATCCGTA | SEQ ID NO: 412 | TAACTTGGAG | SEQ ID NO: 1388 |
| 311 | GCTGAGCGAA | SEQ ID NO: 413 | TCCGGTACGA | SEQ ID NO: 1389 |
| 312 | GTGTTCCAGC | SEQ ID NO: 414 | TTAGTAATCT | SEQ ID NO: 1390 |
| 313 | GAGAAGACGA | SEQ ID NO: 415 | CCGGCCAGTA | SEQ ID NO: 1391 |
| 314 | GCCGGACTGG | SEQ ID NO: 416 | GTCGCTAATC | SEQ ID NO: 1392 |
| 315 | TCGTTCCATC | SEQ ID NO: 417 | AGATATCGAC | SEQ ID NO: 1393 |
| 316 | GCACAGATGT | SEQ ID NO: 418 | CCTCAATAGT | SEQ ID NO: 1394 |
| 317 | CGCGATCAAT | SEQ ID NO: 419 | ACGGTTCACT | SEQ ID NO: 1395 |
| 318 | GTTGGCGCCG | SEQ ID NO: 420 | TATGACATTC | SEQ ID NO: 1396 |
| 319 | ATCTCATCAC | SEQ ID NO: 421 | ATCACAACCG | SEQ ID NO: 1397 |
| 320 | AGTATGATCT | SEQ ID NO: 422 | TAACTCGGCC | SEQ ID NO: 1398 |
| 321 | GTACCACCAT | SEQ ID NO: 423 | TAACGATCTT | SEQ ID NO: 1399 |
| 322 | CTATAACTGG | SEQ ID NO: 424 | ACGAGAACCT | SEQ ID NO: 1400 |
| 323 | TAATCTCATC | SEQ ID NO: 425 | CAGCCATCTA | SEQ ID NO: 1401 |
| 324 | TACTCCGGCG | SEQ ID NO: 426 | ATGGCAATTA | SEQ ID NO: 1402 |
| 325 | CGCTCGATTC | SEQ ID NO: 427 | AGCACCTCTC | SEQ ID NO: 1403 |
| 326 | GTTGCCAGCA | SEQ ID NO: 428 | TTCTATTCGG | SEQ ID NO: 1404 |
| 327 | GGTAGGCCAT | SEQ ID NO: 429 | GGCAAGCACG | SEQ ID NO: 1405 |
| 328 | ACGACGTCAG | SEQ ID NO: 430 | TGGTAACAGC | SEQ ID NO: 1406 |
| 329 | CGTCCACACG | SEQ ID NO: 431 | ATACTAATCA | SEQ ID NO: 1407 |
| 330 | AAGTGCTGGC | SEQ ID NO: 432 | AACGAATCTG | SEQ ID NO: 1408 |
| 331 | CAGCTAAGGA | SEQ ID NO: 433 | GGACAACGCT | SEQ ID NO: 1409 |
| 332 | GTTAACTCAG | SEQ ID NO: 434 | TATTACTATC | SEQ ID NO: 1410 |
| 333 | ACAAGTGTAC | SEQ ID NO: 435 | ACAGCAGGAT | SEQ ID NO: 1411 |
| 334 | GCACGCGATG | SEQ ID NO: 436 | TACTCATTCC | SEQ ID NO: 1412 |
| 335 | TCTCATCCGT | SEQ ID NO: 437 | TTAGTTGCGT | SEQ ID NO: 1413 |
| 336 | GCGGTGGTGG | SEQ ID NO: 438 | GGAAGTCATA | SEQ ID NO: 1414 |
| 337 | TTAGCTAGAG | SEQ ID NO: 439 | ATTCATTGGC | SEQ ID NO: 1415 |
| 338 | TAGTAAGGTG | SEQ ID NO: 440 | ACCAGGTAAG | SEQ ID NO: 1416 |
| 339 | TATCTTAGTG | SEQ ID NO: 441 | AGTAGACAAC | SEQ ID NO: 1417 |
| 340 | CGTAGCTCCG | SEQ ID NO: 442 | CATCCGGTTC | SEQ ID NO: 1418 |
| 341 | ATCGGTAGCC | SEQ ID NO: 443 | CTCCATATTA | SEQ ID NO: 1419 |
| 342 | GCGGCAGAAG | SEQ ID NO: 444 | TTAGAGAAGA | SEQ ID NO: 1420 |
| 343 | GGCGTTGAAG | SEQ ID NO: 445 | TTATCCGTAA | SEQ ID NO: 1421 |
| 344 | TTACAGCTAT | SEQ ID NO: 446 | ACGCTAATAT | SEQ ID NO: 1422 |
| 345 | TCGTTGGTCC | SEQ ID NO: 447 | AATACGTTGT | SEQ ID NO: 1423 |
| 346 | GAATGTTGAA | SEQ ID NO: 448 | TCGGCTGATG | SEQ ID NO: 1424 |
| 347 | CGCTACCACT | SEQ ID NO: 449 | TTGTACTAGG | SEQ ID NO: 1425 |
| 348 | TCGTCCAGCA | SEQ ID NO: 450 | AGTTAAGGTC | SEQ ID NO: 1426 |
| 349 | GAGTACAGCC | SEQ ID NO: 451 | CTTCCAGGCA | SEQ ID NO: 1427 |
| 350 | GAGTTAGAAT | SEQ ID NO: 452 | CCGAATAGGC | SEQ ID NO: 1428 |
| 351 | CAGTGTGAGA | SEQ ID NO: 453 | ACCTTGGTAA | SEQ ID NO: 1429 |
| 352 | AGAGTTCTGG | SEQ ID NO: 454 | TGATCCTACT | SEQ ID NO: 1430 |
| 353 | GCACCTATGG | SEQ ID NO: 455 | TGGAACGCTC | SEQ ID NO: 1431 |
| 354 | TTGCGTTCTC | SEQ ID NO: 456 | CCGTTCACCG | SEQ ID NO: 1432 |
| 355 | TGTACAGAAG | SEQ ID NO: 457 | ACAGTCATTG | SEQ ID NO: 1433 |
| 356 | GGCGTCATTC | SEQ ID NO: 458 | TCACCATTCT | SEQ ID NO: 1434 |
| 357 | CATATCAGGT | SEQ ID NO: 459 | GGTTCCACTT | SEQ ID NO: 1435 |
| 358 | GTATGTCCGC | SEQ ID NO: 460 | TCCTGTGCCG | SEQ ID NO: 1436 |
| 359 | TGCGGCTACC | SEQ ID NO: 461 | TGTTGTGCAT | SEQ ID NO: 1437 |
| 360 | GGCCTGCGAC | SEQ ID NO: 462 | TGAGCTATAA | SEQ ID NO: 1438 |
| 361 | AGCTCCTGCA | SEQ ID NO: 463 | AGTTGCCGGT | SEQ ID NO: 1439 |
| 362 | GCGGTACTGC | SEQ ID NO: 464 | GAGATCACGG | SEQ ID NO: 1440 |
| 363 | CGCGAATGCC | SEQ ID NO: 465 | ACGGCCATAG | SEQ ID NO: 1441 |
| 364 | CCTACAGCGG | SEQ ID NO: 466 | CTCCTCAGTA | SEQ ID NO: 1442 |
| 365 | TATCCTAATT | SEQ ID NO: 467 | CGCCGCAGAG | SEQ ID NO: 1443 |
| 366 | GACACTATTG | SEQ ID NO: 468 | TTGTAACATT | SEQ ID NO: 1444 |
| 367 | TCTATATGAC | SEQ ID NO: 469 | CTAGTGTACC | SEQ ID NO: 1445 |
| 368 | GTTGTGCAGT | SEQ ID NO: 470 | TTATCGCTAG | SEQ ID NO: 1446 |
| 369 | TTAGGCAACT | SEQ ID NO: 471 | GATCAGTATA | SEQ ID NO: 1447 |
| 370 | GCTTACGCGG | SEQ ID NO: 472 | TGGCCATACC | SEQ ID NO: 1448 |
| 371 | GCTAGTCTCA | SEQ ID NO: 473 | TATTCCTCAC | SEQ ID NO: 1449 |
| 372 | GTCGGTGATG | SEQ ID NO: 474 | TGAGATGTGA | SEQ ID NO: 1450 |
| 373 | GAGGAACCTT | SEQ ID NO: 475 | GGCGCAACAA | SEQ ID NO: 1451 |
| 374 | AGCGGAATAA | SEQ ID NO: 476 | CCATGATCGA | SEQ ID NO: 1452 |
| 375 | CTAATGATAC | SEQ ID NO: 477 | CTCTACCTGC | SEQ ID NO: 1453 |
| 376 | TAGCGGCGCT | SEQ ID NO: 478 | TCATCTGGCA | SEQ ID NO: 1454 |
| 377 | GCGGTCTTGA | SEQ ID NO: 479 | GTCGCTGCCT | SEQ ID NO: 1455 |
| 378 | CGCGCTGAGT | SEQ ID NO: 480 | TAACCACCGA | SEQ ID NO: 1456 |
| 379 | CACGGACAGG | SEQ ID NO: 481 | GGACCGCACA | SEQ ID NO: 1457 |
| 380 | GTGCGTACTA | SEQ ID NO: 482 | CCACGTAACA | SEQ ID NO: 1458 |
| 381 | TAGTGTGCGG | SEQ ID NO: 483 | AGTAAGAAGA | SEQ ID NO: 1459 |
| 382 | CGATCTTAGA | SEQ ID NO: 484 | AACAGCATGG | SEQ ID NO: 1460 |
| 383 | GACGGTCAGT | SEQ ID NO: 485 | TAAGCGAGCA | SEQ ID NO: 1461 |
| 384 | TGCCGGCCAT | SEQ ID NO: 486 | TAGCGAGAAC | SEQ ID NO: 1462 |
| 385 | GTTGTCAGTG | SEQ ID NO: 487 | GATCACCTAG | SEQ ID NO: 1463 |
| 386 | GTACCTTGAG | SEQ ID NO: 488 | TGCACACACC | SEQ ID NO: 1464 |
| 387 | GTATTGCTCT | SEQ ID NO: 489 | TGGATCCGAA | SEQ ID NO: 1465 |
| 388 | TAACGTTGCT | SEQ ID NO: 490 | AGAGACCTGC | SEQ ID NO: 1466 |
| 389 | CTCCGCATGA | SEQ ID NO: 491 | CATATTACGA | SEQ ID NO: 1467 |
| 390 | AATACTGCGT | SEQ ID NO: 492 | CCGACTACAG | SEQ ID NO: 1468 |
| 391 | TTGCTTATGC | SEQ ID NO: 493 | TTCGGCTGAG | SEQ ID NO: 1469 |
| 392 | CACCTCTCGG | SEQ ID NO: 494 | ACGGACAGCT | SEQ ID NO: 1470 |
| 393 | CTTGCTCAGT | SEQ ID NO: 495 | ACCTAGTCCT | SEQ ID NO: 1471 |
| 394 | ATCAGGTGAA | SEQ ID NO: 496 | GGAGCAGAGA | SEQ ID NO: 1472 |
| 395 | GTACTTACGT | SEQ ID NO: 497 | TGTCAATAGC | SEQ ID NO: 1473 |
| 396 | GTCGCCGGTG | SEQ ID NO: 498 | CGCAATGCTA | SEQ ID NO: 1474 |
| 397 | AATAGATTAT | SEQ ID NO: 499 | AGCCACTGGC | SEQ ID NO: 1475 |
| 398 | AAGAGTACCG | SEQ ID NO: 500 | AATTCCAATG | SEQ ID NO: 1476 |
| 399 | GAGATACCGT | SEQ ID NO: 501 | TCGCCGTCCA | SEQ ID NO: 1477 |
| 400 | CTGATGTAAC | SEQ ID NO: 502 | CGACATGAAG | SEQ ID NO: 1478 |
| 401 | CAGAGTTCGA | SEQ ID NO: 503 | AGTCATGCAG | SEQ ID NO: 1479 |
| 402 | GATGACATAT | SEQ ID NO: 504 | GGCAGCTGTA | SEQ ID NO: 1480 |
| 403 | TGTCCGTAGG | SEQ ID NO: 505 | ACCGCAGATG | SEQ ID NO: 1481 |
| 404 | CACGTCTAAT | SEQ ID NO: 506 | CAATCGCACA | SEQ ID NO: 1482 |
| 405 | AGCCGTGGTC | SEQ ID NO: 507 | TTGACGCTTC | SEQ ID NO: 1483 |
| 406 | TGTGGTCTCA | SEQ ID NO: 508 | ATTGGTGGTT | SEQ ID NO: 1484 |
| 407 | GAATCCGGAA | SEQ ID NO: 509 | CAACGAAGAT | SEQ ID NO: 1485 |
| 408 | TGTCGGACCA | SEQ ID NO: 510 | AGTATTGCTT | SEQ ID NO: 1486 |
| 409 | AGGTCTGCCG | SEQ ID NO: 511 | GTAAGTATGA | SEQ ID NO: 1487 |
| 410 | CTAGCGGTGG | SEQ ID NO: 512 | ATATGTATCA | SEQ ID NO: 1488 |
| 411 | ACGTTAGTCA | SEQ ID NO: 513 | CATCAAGTAC | SEQ ID NO: 1489 |
| 412 | GAATATTGGT | SEQ ID NO: 514 | TCGATAGCAT | SEQ ID NO: 1490 |
| 413 | GTAAGGCAAC | SEQ ID NO: 515 | GAATCATTGA | SEQ ID NO: 1491 |
| 414 | GACTGCGACA | SEQ ID NO: 516 | CTTGGTTGGA | SEQ ID NO: 1492 |
| 415 | TTATGAACAT | SEQ ID NO: 517 | ACGGTTATGG | SEQ ID NO: 1493 |
| 416 | AACGTCATAT | SEQ ID NO: 518 | CCGTCGCATA | SEQ ID NO: 1494 |
| 417 | GGCGTTCGCT | SEQ ID NO: 519 | GCACACGACC | SEQ ID NO: 1495 |
| 418 | TAGTGTACAT | SEQ ID NO: 520 | ACCTATTCAA | SEQ ID NO: 1496 |
| 419 | GGATCGGCAG | SEQ ID NO: 521 | TAGAGATGAG | SEQ ID NO: 1497 |
| 420 | GTCCGGCTTG | SEQ ID NO: 522 | CTCACGATAG | SEQ ID NO: 1498 |
| 421 | ACCGTGCGGC | SEQ ID NO: 523 | AGACGAGATT | SEQ ID NO: 1499 |
| 422 | TGACTGGCGT | SEQ ID NO: 524 | AACTCCACCG | SEQ ID NO: 1500 |
| 423 | TATCGCGCAC | SEQ ID NO: 525 | CGGCAGCCTC | SEQ ID NO: 1501 |
| 424 | TCGAACGAGT | SEQ ID NO: 526 | ACCACAGAGT | SEQ ID NO: 1502 |
| 425 | AAGGAGCAAT | SEQ ID NO: 527 | ACTAGGACGA | SEQ ID NO: 1503 |
| 426 | GATCGTTCTA | SEQ ID NO: 528 | CCTACGTTCC | SEQ ID NO: 1504 |
| 427 | ATACCTCTGG | SEQ ID NO: 529 | TATTCTTCCG | SEQ ID NO: 1505 |
| 428 | GTGCGCCGTA | SEQ ID NO: 530 | CCTCCTCTGG | SEQ ID NO: 1506 |
| 429 | CGTATTAGCC | SEQ ID NO: 531 | CGGACGTATG | SEQ ID NO: 1507 |
| 430 | TGCGCTCGTA | SEQ ID NO: 532 | GGAACGTAGA | SEQ ID NO: 1508 |
| 431 | ACTAGTTGAA | SEQ ID NO: 533 | ATTGGTATGT | SEQ ID NO: 1509 |
| 432 | GTGGCTCTGT | SEQ ID NO: 534 | TCCGCTTAAT | SEQ ID NO: 1510 |
| 433 | GCCAACGGAT | SEQ ID NO: 535 | TGCAATGCAT | SEQ ID NO: 1511 |
| 434 | GGCAACTTAT | SEQ ID NO: 536 | CTGGCAGCGC | SEQ ID NO: 1512 |
| 435 | CATTAATCTC | SEQ ID NO: 537 | TTCCGCATAG | SEQ ID NO: 1513 |
| 436 | CGCGACACTA | SEQ ID NO: 538 | AACTACAGCA | SEQ ID NO: 1514 |
| 437 | GAGGAATCGC | SEQ ID NO: 539 | GACCTGACCA | SEQ ID NO: 1515 |
| 438 | AGGTGTGATC | SEQ ID NO: 540 | GACAGATTAA | SEQ ID NO: 1516 |
| 439 | AACTCGGACG | SEQ ID NO: 541 | ATCCTCCTGA | SEQ ID NO: 1517 |
| 440 | TTCATGGCGT | SEQ ID NO: 542 | AATCCAATCT | SEQ ID NO: 1518 |
| 441 | TCACTCGTTG | SEQ ID NO: 543 | ACCGGCTACT | SEQ ID NO: 1519 |
| 442 | TGGACTCCGT | SEQ ID NO: 544 | ATTGGCTAGA | SEQ ID NO: 1520 |
| 443 | CTCGGTGCCG | SEQ ID NO: 545 | AATGAGATTG | SEQ ID NO: 1521 |
| 444 | CCTGCAGCAA | SEQ ID NO: 546 | ACTCCTGATG | SEQ ID NO: 1522 |
| 445 | AAGTCGTAAG | SEQ ID NO: 547 | ATCATAATGA | SEQ ID NO: 1523 |
| 446 | GATGCTAGAT | SEQ ID NO: 548 | CTCCTGTTCG | SEQ ID NO: 1524 |
| 447 | GTACTGAGTT | SEQ ID NO: 549 | CATGCCTGGC | SEQ ID NO: 1525 |
| 448 | CTCCGGTCCT | SEQ ID NO: 550 | ATAAGTTCAC | SEQ ID NO: 1526 |
| 449 | TTCACGGATG | SEQ ID NO: 551 | TTAAGACACC | SEQ ID NO: 1527 |
| 450 | CGTAGTGGAT | SEQ ID NO: 552 | CGGCACAGAC | SEQ ID NO: 1528 |
| 451 | GCGTCAGTAT | SEQ ID NO: 553 | TCATGCAACG | SEQ ID NO: 1529 |
| 452 | TGAGTGTTCT | SEQ ID NO: 554 | ATCCGATTAG | SEQ ID NO: 1530 |
| 453 | GTCGCTTCTA | SEQ ID NO: 555 | CAACATCCGA | SEQ ID NO: 1531 |
| 454 | TCTATTGATG | SEQ ID NO: 556 | ATCCTATTCT | SEQ ID NO: 1532 |
| 455 | GGTGTAGTTA | SEQ ID NO: 557 | TGTCGAAGTT | SEQ ID NO: 1533 |
| 456 | TAAGGACTGG | SEQ ID NO: 558 | ACCAAGACCG | SEQ ID NO: 1534 |
| 457 | GTCCACCGGA | SEQ ID NO: 559 | TCGGCACCTG | SEQ ID NO: 1535 |
| 458 | ACGCGTTACC | SEQ ID NO: 560 | CTCGAAGCCT | SEQ ID NO: 1536 |
| 459 | CAGGACGTAC | SEQ ID NO: 561 | CATAGTTAGG | SEQ ID NO: 1537 |
| 460 | AGGTGACCTG | SEQ ID NO: 562 | GGTTGTACTA | SEQ ID NO: 1538 |
| 461 | GTTACTCATA | SEQ ID NO: 563 | TTAGGAGCCG | SEQ ID NO: 1539 |
| 462 | CACGCGGTTA | SEQ ID NO: 564 | AATGGCATGC | SEQ ID NO: 1540 |
| 463 | GACTATGCTG | SEQ ID NO: 565 | TGATGATTCC | SEQ ID NO: 1541 |
| 464 | GAAGAGTGCT | SEQ ID NO: 566 | TATAGCCGCA | SEQ ID NO: 1542 |
| 465 | TGTCCGTCTA | SEQ ID NO: 567 | ATTAAGTACC | SEQ ID NO: 1543 |
| 466 | TGTTGATGGC | SEQ ID NO: 568 | CACTATTGAT | SEQ ID NO: 1544 |
| 467 | ACCATGGACG | SEQ ID NO: 569 | AGAGCCTTGA | SEQ ID NO: 1545 |
| 468 | GCACAGGCGA | SEQ ID NO: 570 | GGCGGCGGTT | SEQ ID NO: 1546 |
| 469 | TGATCAGGTT | SEQ ID NO: 571 | GTATCCTTCG | SEQ ID NO: 1547 |
| 470 | GAGAGGTCCG | SEQ ID NO: 572 | GTGCCGCTAA | SEQ ID NO: 1548 |
| 471 | AGGCCACGAT | SEQ ID NO: 573 | ATTACGAAGG | SEQ ID NO: 1549 |
| 472 | CCTTGGTGCA | SEQ ID NO: 574 | AACTGATTGA | SEQ ID NO: 1550 |
| 473 | CCTTATGATC | SEQ ID NO: 575 | ATAGCTTCCA | SEQ ID NO: 1551 |
| 474 | GCGTCTAACC | SEQ ID NO: 576 | TGTATCATCA | SEQ ID NO: 1552 |
| 475 | CTAGACGATG | SEQ ID NO: 577 | TAACCATTGG | SEQ ID NO: 1553 |
| 476 | CCTGCGCGGA | SEQ ID NO: 578 | TAATAGCTGC | SEQ ID NO: 1554 |
| 477 | AGGCGCTGAA | SEQ ID NO: 579 | GACAATGGCA | SEQ ID NO: 1555 |
| 478 | CGTTCCGTTA | SEQ ID NO: 580 | CTCATCCGTT | SEQ ID NO: 1556 |
| 479 | ATCGAAGTAT | SEQ ID NO: 581 | CTCTCAGCGG | SEQ ID NO: 1557 |
| 480 | ATACCAATAC | SEQ ID NO: 582 | ACATATCATG | SEQ ID NO: 1558 |
| 481 | GAGTGCATCG | SEQ ID NO: 583 | GCCAATCGAC | SEQ ID NO: 1559 |
| 482 | GCTGACTCCG | SEQ ID NO: 584 | GCCTACGGTG | SEQ ID NO: 1560 |
| 483 | GTTGCGTCTT | SEQ ID NO: 585 | CCGATCATAG | SEQ ID NO: 1561 |
| 484 | TATGGCCTCC | SEQ ID NO: 586 | ATTACTAGAC | SEQ ID NO: 1562 |
| 485 | GGTGTATGGC | SEQ ID NO: 587 | CGGAGAAGTG | SEQ ID NO: 1563 |
| 486 | GTCGTAGCAA | SEQ ID NO: 588 | TCGCTGAGTG | SEQ ID NO: 1564 |
| 487 | GAAGATCCTC | SEQ ID NO: 589 | GGAATACTCT | SEQ ID NO: 1565 |
| 488 | GTCCTCGCGG | SEQ ID NO: 590 | GCATTGGTCA | SEQ ID NO: 1566 |
| 489 | TTCGAACTCC | SEQ ID NO: 591 | ATCGCCTGAT | SEQ ID NO: 1567 |
| 490 | TATGGCAGCG | SEQ ID NO: 592 | ATTCAAGCAC | SEQ ID NO: 1568 |
| 491 | CTCACAAGGC | SEQ ID NO: 593 | GGCGGCAAGC | SEQ ID NO: 1569 |
| 492 | GGACGTGCGC | SEQ ID NO: 594 | TATAGAATGT | SEQ ID NO: 1570 |
| 493 | CACTCCGTTG | SEQ ID NO: 595 | CCACAGCGAC | SEQ ID NO: 1571 |
| 494 | GCCGTGATCT | SEQ ID NO: 596 | TACGAATGCA | SEQ ID NO: 1572 |
| 495 | AACTCCGGAT | SEQ ID NO: 597 | AGCCATCATA | SEQ ID NO: 1573 |
| 496 | TGCGGAGCGG | SEQ ID NO: 598 | AGCTGACTGC | SEQ ID NO: 1574 |
| 497 | GTCGGCTGCA | SEQ ID NO: 599 | GGTGGACCTG | SEQ ID NO: 1575 |
| 498 | CAATAGGAGA | SEQ ID NO: 600 | GGCTTAACCA | SEQ ID NO: 1576 |
| 499 | CTGTGACGGT | SEQ ID NO: 601 | GGAGCCTAAT | SEQ ID NO: 1577 |
| 500 | CCACGCGGCT | SEQ ID NO: 602 | ACAAGAGCAG | SEQ ID NO: 1578 |
| 501 | TCGGAGAGCC | SEQ ID NO: 603 | CGCCTATGAA | SEQ ID NO: 1579 |
| 502 | GAAGGCACGA | SEQ ID NO: 604 | GGTCGCTAAT | SEQ ID NO: 1580 |
| 503 | CTCTGCTCGG | SEQ ID NO: 605 | CTCTATCACG | SEQ ID NO: 1581 |
| 504 | GCTCCAGGCC | SEQ ID NO: 606 | GACATGACAC | SEQ ID NO: 1582 |
| 505 | GCTCGCGCCT | SEQ ID NO: 607 | TCTAAGTAAG | SEQ ID NO: 1583 |
| 506 | GTGGAGAGAT | SEQ ID NO: 608 | TTGTGCTTAG | SEQ ID NO: 1584 |
| 507 | CTGCGCGCCG | SEQ ID NO: 609 | TTCTAGTGCC | SEQ ID NO: 1585 |
| 508 | AGACATAGGT | SEQ ID NO: 610 | ACTTAGGACT | SEQ ID NO: 1586 |
| 509 | AATGAGTCAT | SEQ ID NO: 611 | TAAGCCATCT | SEQ ID NO: 1587 |
| 510 | TTGCTATCCG | SEQ ID NO: 612 | CTAGACTTCT | SEQ ID NO: 1588 |
| 511 | TCATGAGCTT | SEQ ID NO: 613 | AGTATCTATT | SEQ ID NO: 1589 |
| 512 | GCGCATGACT | SEQ ID NO: 614 | CTGGTTGTAA | SEQ ID NO: 1590 |
| 513 | TCCATATGTT | SEQ ID NO: 615 | CCGCAGACCT | SEQ ID NO: 1591 |
| 514 | CGAGTCCGAA | SEQ ID NO: 616 | CCGCACCAAC | SEQ ID NO: 1592 |
| 515 | CTCGAGCAGA | SEQ ID NO: 617 | AGTTGGCAGC | SEQ ID NO: 1593 |
| 516 | GCGTTAGTTG | SEQ ID NO: 618 | TTCGGCTCCT | SEQ ID NO: 1594 |
| 517 | ATAATCTAGA | SEQ ID NO: 619 | CAATGTATGG | SEQ ID NO: 1595 |
| 518 | ATCTGTCCTT | SEQ ID NO: 620 | CAACTATATA | SEQ ID NO: 1596 |
| 519 | GAACCGCGCG | SEQ ID NO: 621 | CCACTTGTGC | SEQ ID NO: 1597 |
| 520 | GTGATTCGGA | SEQ ID NO: 622 | CAAGGCGACT | SEQ ID NO: 1598 |
| 52 | GCTCAGAGTA | SEQ ID NO: 623 | GCAACCTGCA | SEQ ID NO: 1599 |
| 522 | GAAGACAGTT | SEQ ID NO: 624 | GCAGCCGCGC | SEQ ID NO: 1600 |
| 523 | TATGTATCGC | SEQ ID NO: 625 | ATTGTGCCTG | SEQ ID NO: 1601 |
| 524 | GTCTGTTGCC | SEQ ID NO: 626 | TCCATGAGAG | SEQ ID NO: 1602 |
| 525 | TCCGTAGAGG | SEQ ID NO: 627 | ATTGATTAGG | SEQ ID NO: 1603 |
| 526 | TGAGTACGTG | SEQ ID NO: 628 | CGCCATGATT | SEQ ID NO: 1604 |
| 527 | TGTCCTGTGT | SEQ ID NO: 629 | ACGGATTAAG | SEQ ID NO: 1605 |
| 528 | GCGACGGCCG | SEQ ID NO: 630 | GTAGAAGTTG | SEQ ID NO: 1606 |
| 529 | TGCCTGAGGT | SEQ ID NO: 631 | AAGGTTCCGC | SEQ ID NO: 1607 |
| 530 | TACATCCTAT | SEQ ID NO: 632 | CGCTCAGCCT | SEQ ID NO: 1608 |
| 531 | GCGCTGCCGT | SEQ ID NO: 633 | TCACATGTAA | SEQ ID NO: 1609 |
| 532 | GCTTGCGGCC | SEQ ID NO: 634 | GAGATCCTGA | SEQ ID NO: 1610 |
| 533 | GCTTCTTCAT | SEQ ID NO: 635 | GTTGTATTAT | SEQ ID NO: 1611 |
| 534 | GTTATTAAGG | SEQ ID NO: 636 | GGACCTATCC | SEQ ID NO: 1612 |
| 535 | TCGTGAGTGG | SEQ ID NO: 637 | AACCTCGTAA | SEQ ID NO: 1613 |
| 536 | CTGTAACGTA | SEQ ID NO: 638 | CGCAGCTACT | SEQ ID NO: 1614 |
| 537 | CACATCACCA | SEQ ID NO: 639 | CATCTTCATT | SEQ ID NO: 1615 |
| 538 | GCAGTCCTAG | SEQ ID NO: 640 | GTGGTCCTCG | SEQ ID NO: 1616 |
| 539 | CCTTGGCGAG | SEQ ID NO: 641 | AAGAATGTAG | SEQ ID NO: 1617 |
| 540 | CGCGGTCTTG | SEQ ID NO: 642 | ACGACTTGTT | SEQ ID NO: 1618 |
| 541 | CTGCGTCAAG | SEQ ID NO: 643 | TCAATAGCTC | SEQ ID NO: 1619 |
| 542 | AGGATACATA | SEQ ID NO: 644 | GTGGCATTCT | SEQ ID NO: 1620 |
| 543 | CTGAGTTGTC | SEQ ID NO: 645 | CAGCATCTGC | SEQ ID NO: 1621 |
| 544 | GCGGCGAGTT | SEQ ID NO: 646 | GGTAGAGGTC | SEQ ID NO: 1622 |
| 545 | GGTCTTACCT | SEQ ID NO: 647 | CGGACTAGCT | SEQ ID NO: 1623 |
| 546 | TACTCTCCTG | SEQ ID NO: 648 | ACTATCTCTA | SEQ ID NO: 1624 |
| 547 | CGCTCTATGA | SEQ ID NO: 649 | ATTCGCATTG | SEQ ID NO: 1625 |
| 548 | TTGAGGCATT | SEQ ID NO: 650 | GACTTCCAGG | SEQ ID NO: 1626 |
| 549 | GTAGGCGTTC | SEQ ID NO: 651 | GGCTTGTAAG | SEQ ID NO: 1627 |
| 550 | CTCGCTAGGT | SEQ ID NO: 652 | GTTCACGATT | SEQ ID NO: 1628 |
| 551 | GCAGGTTCTA | SEQ ID NO: 653 | GGTTGACATT | SEQ ID NO: 1629 |
| 552 | GGTCGTAGAA | SEQ ID NO: 654 | GAATCGTAGC | SEQ ID NO: 1630 |
| 553 | GGTTGTCTCC | SEQ ID NO: 655 | TGATGCCGCC | SEQ ID NO: 1631 |
| 554 | CACATGTCGC | SEQ ID NO: 656 | TACCAACTGC | SEQ ID NO: 1632 |
| 555 | GTCGTCCGGT | SEQ ID NO: 657 | CATAGCCGTC | SEQ ID NO: 1633 |
| 556 | GTGGAAGTAA | SEQ ID NO: 658 | GTGGCCTCGC | SEQ ID NO: 1634 |
| 557 | GCACGTACAT | SEQ ID NO: 659 | GTCATTGGAT | SEQ ID NO: 1635 |
| 558 | TCGAGTATGC | SEQ ID NO: 660 | TCAGAGGTAG | SEQ ID NO: 1636 |
| 559 | AGCTCGTAGT | SEQ ID NO: 661 | GTTACCGTCC | SEQ ID NO: 1637 |
| 560 | CTCCGTTATC | SEQ ID NO: 662 | CGGTAGACGC | SEQ ID NO: 1638 |
| 561 | CCTCTACTTG | SEQ ID NO: 663 | ATTCGGAGAC | SEQ ID NO: 1639 |
| 562 | GGTGGCGTCT | SEQ ID NO: 664 | TGGACAAGCG | SEQ ID NO: 1640 |
| 563 | CGCCGAGTCA | SEQ ID NO: 665 | ATAGCAATGG | SEQ ID NO: 1641 |
| 564 | GTCTGCCACT | SEQ ID NO: 666 | TCGCTGTTAG | SEQ ID NO: 1642 |
| 565 | GCGTTCGACG | SEQ ID NO: 667 | CTCTAGCCGT | SEQ ID NO: 1643 |
| 566 | CAGTCTTGTT | SEQ ID NO: 668 | GTCATCGCTT | SEQ ID NO: 1644 |
| 567 | GGTATCTCCT | SEQ ID NO: 669 | CCAAGTCTGC | SEQ ID NO: 1645 |
| 568 | CTGTACTCAC | SEQ ID NO: 670 | TCTCACCGCA | SEQ ID NO: 1646 |
| 569 | TTACGCGTGA | SEQ ID NO: 671 | ACCGATCCAT | SEQ ID NO: 1647 |
| 570 | AGGTTCTCGT | SEQ ID NO: 672 | GGCCTTCAGC | SEQ ID NO: 1648 |
| 571 | CTTGCGATCC | SEQ ID NO: 673 | TGTGAACGAT | SEQ ID NO: 1649 |
| 572 | TGAATCGTGG | SEQ ID NO: 674 | ATACCGTATG | SEQ ID NO: 1650 |
| 573 | TCGACGTGGA | SEQ ID NO: 675 | TGGAGTGGTG | SEQ ID NO: 1651 |
| 574 | GGCAAGGTAC | SEQ ID NO: 676 | GAACTATCAC | SEQ ID NO: 1652 |
| 575 | CTCAGCTGCC | SEQ ID NO: 677 | TACACTTGTC | SEQ ID NO: 1653 |
| 576 | GCCTGTCAGA | SEQ ID NO: 678 | TCATCTATCC | SEQ ID NO: 1654 |
| 577 | AGCGACATCA | SEQ ID NO: 679 | ATTAATATCT | SEQ ID NO: 1655 |
| 578 | GCGAGAATAT | SEQ ID NO: 680 | CAATGCTTAA | SEQ ID NO: 1656 |
| 579 | GGCTAGCTCA | SEQ ID NO: 681 | TCACATTCTA | SEQ ID NO: 1657 |
| 580 | TATTCGGTAC | SEQ ID NO: 682 | TTCCAGCAAC | SEQ ID NO: 1658 |
| 581 | TTGGTAGGAC | SEQ ID NO: 683 | GGACGGCATC | SEQ ID NO: 1659 |
| 582 | CAATCGTGGT | SEQ ID NO: 684 | AACGTAACTC | SEQ ID NO: 1660 |
| 583 | CGCTGGCGCG | SEQ ID NO: 685 | ATGGTCCATC | SEQ ID NO: 1661 |
| 584 | CTGGTGCGTT | SEQ ID NO: 686 | GACAATCCGT | SEQ ID NO: 1662 |
| 585 | GCGACGCTAG | SEQ ID NO: 687 | GGAATCCGAT | SEQ ID NO: 1663 |
| 586 | GCGCTGGTCT | SEQ ID NO: 688 | TCCTCGAGTC | SEQ ID NO: 1664 |
| 587 | TGTCTTCTAA | SEQ ID NO: 689 | TCGAAGAGTA | SEQ ID NO: 1665 |
| 588 | TCATACCGGT | SEQ ID NO: 690 | AGCGCGGCAA | SEQ ID NO: 1666 |
| 589 | GCTTCGTGGC | SEQ ID NO: 691 | TAACCGACCG | SEQ ID NO: 1667 |
| 590 | TGGAGCACAT | SEQ ID NO: 692 | CCATCCTGGA | SEQ ID NO: 1668 |
| 591 | GGCTATCAAC | SEQ ID NO: 693 | CTGCAACCAA | SEQ ID NO: 1669 |
| 592 | TTATTACGTA | SEQ ID NO: 694 | CCAGCTGCCT | SEQ ID NO: 1670 |
| 593 | AGGCAGCTAC | SEQ ID NO: 695 | GACGCACTAT | SEQ ID NO: 1671 |
| 594 | GCTGTCGGCG | SEQ ID NO: 696 | GTCCACGGCT | SEQ ID NO: 1672 |
| 595 | ATACTGTGGC | SEQ ID NO: 697 | CTCAGCACTA | SEQ ID NO: 1673 |
| 596 | ATGAAGACGG | SEQ ID NO: 698 | AGTAACGGTG | SEQ ID NO: 1674 |
| 597 | ATCGTCTTAA | SEQ ID NO: 699 | TCCAGCAATG | SEQ ID NO: 1675 |
| 598 | AATGTCTGTA | SEQ ID NO: 700 | CCAACCATGC | SEQ ID NO: 1676 |
| 599 | GGTCAGCGTG | SEQ ID NO: 701 | TTCGGTCAAT | SEQ ID NO: 1677 |
| 600 | TTAGGTCCTA | SEQ ID NO: 702 | AATCAGGTCT | SEQ ID NO: 1678 |
| 601 | GACCGTGAAT | SEQ ID NO: 703 | TACGTGGACG | SEQ ID NO: 1679 |
| 602 | ACTTCTGTCC | SEQ ID NO: 704 | CCTGTGTCGA | SEQ ID NO: 1680 |
| 603 | ATCGGCGAAC | SEQ ID NO: 705 | CTCGAGTGTA | SEQ ID NO: 1681 |
| 604 | GCAAGCTTAT | SEQ ID NO: 706 | TGGATCCTTC | SEQ ID NO: 1682 |
| 605 | TAGCTCAGGC | SEQ ID NO: 707 | TAGGTAGAGT | SEQ ID NO: 1683 |
| 606 | GCTGTTGCTG | SEQ ID NO: 708 | GACTTGTGTC | SEQ ID NO: 1684 |
| 607 | GTGAATGGAG | SEQ ID NO: 709 | CTTGAACTTA | SEQ ID NO: 1685 |
| 608 | GTCTAAGCAC | SEQ ID NO: 710 | TCAAGCCGAG | SEQ ID NO: 1686 |
| 609 | ATAGCGCGAT | SEQ ID NO: 711 | TATGGACCAG | SEQ ID NO: 1687 |
| 610 | GCTGAGGATA | SEQ ID NO: 712 | GACCTTACTT | SEQ ID NO: 1688 |
| 611 | ATCTCCTAAG | SEQ ID NO: 713 | CGGCTCGGCG | SEQ ID NO: 1689 |
| 612 | GTCCGAGCAG | SEQ ID NO: 714 | TCGCATGAAG | SEQ ID NO: 1690 |
| 613 | TCGAGGTGAT | SEQ ID NO: 715 | GGACGCATTA | SEQ ID NO: 1691 |
| 614 | GATACGTGCG | SEQ ID NO: 716 | TGAACAACTT | SEQ ID NO: 1692 |
| 615 | ATTGTATACT | SEQ ID NO: 717 | ACCACTGGCT | SEQ ID NO: 1693 |
| 616 | CGTTAACTGA | SEQ ID NO: 718 | AGTGAGCTGT | SEQ ID NO: 1694 |
| 617 | ACTCGTATGC | SEQ ID NO: 719 | TCCGTTCGTT | SEQ ID NO: 1695 |
| 618 | GTCCTGTCAA | SEQ ID NO: 720 | TCTCCACAAC | SEQ ID NO: 1696 |
| 619 | TAGATCGTCC | SEQ ID NO: 721 | ATAGTGAATC | SEQ ID NO: 1697 |
| 620 | CGTCCGTGGT | SEQ ID NO: 722 | CCTTGCTAGA | SEQ ID NO: 1698 |
| 621 | TACTGTCTGT | SEQ ID NO: 723 | CGATGCCACG | SEQ ID NO: 1699 |
| 622 | GTGGTACACA | SEQ ID NO: 724 | TGACTCCGGC | SEQ ID NO: 1700 |
| 623 | CGACCGACGT | SEQ ID NO: 725 | AACATTAGGA | SEQ ID NO: 1701 |
| 624 | TCGTGCCTAT | SEQ ID NO: 726 | CCATCGTCAA | SEQ ID NO: 1702 |
| 625 | GCATGGCTAG | SEQ ID NO: 727 | CTGACACTCC | SEQ ID NO: 1703 |
| 626 | ATCCGTAGGA | SEQ ID NO: 728 | GCCATCAACA | SEQ ID NO: 1704 |
| 627 | CTCTAAGAGA | SEQ ID NO: 729 | ATTCTAGTAG | SEQ ID NO: 1705 |
| 628 | CCTCCTTAAG | SEQ ID NO: 730 | ATATCGCACG | SEQ ID NO: 1706 |
| 629 | AATTACGTTA | SEQ ID NO: 731 | AAGATCCGAC | SEQ ID NO: 1707 |
| 630 | GCAGTCACGT | SEQ ID NO: 732 | CCGTATTCGA | SEQ ID NO: 1708 |
| 631 | AAGGCGCATC | SEQ ID NO: 733 | GCATACCTCG | SEQ ID NO: 1709 |
| 632 | CTGGATGGCG | SEQ ID NO: 734 | CGACGACCTG | SEQ ID NO: 1710 |
| 633 | CTAAGGTCGA | SEQ ID NO: 735 | GTCATAAGAA | SEQ ID NO: 1711 |
| 634 | AAGATGAGGT | SEQ ID NO: 736 | GTCAGACGCT | SEQ ID NO: 1712 |
| 635 | GAGTCGCAGT | SEQ ID NO: 737 | TCGAGCTAGC | SEQ ID NO: 1713 |
| 636 | CGGCGTTGTT | SEQ ID NO: 738 | CATACCAGCG | SEQ ID NO: 1714 |
| 637 | GGAGTGACTC | SEQ ID NO: 739 | CACGCACATA | SEQ ID NO: 1715 |
| 638 | CGTAGTGTTG | SEQ ID NO: 740 | CCTCGGTGAC | SEQ ID NO: 1716 |
| 639 | CGTCTGCATA | SEQ ID NO: 741 | CCGTTCGATT | SEQ ID NO: 1717 |
| 640 | CGATACAAGG | SEQ ID NO: 742 | AATTAGTAGG | SEQ ID NO: 1718 |
| 641 | CGCGCGTTGC | SEQ ID NO: 743 | ACACCTGCGT | SEQ ID NO: 1719 |
| 642 | TAGAGGCGGA | SEQ ID NO: 744 | CGCACCAAGG | SEQ ID NO: 1720 |
| 643 | ATTCTCCGTT | SEQ ID NO: 745 | CTTCGTACCA | SEQ ID NO: 1721 |
| 644 | CCAGCGTATC | SEQ ID NO: 746 | TTCCGACATC | SEQ ID NO: 1722 |
| 645 | AGAACTAGGC | SEQ ID NO: 747 | GATGACAACA | SEQ ID NO: 1723 |
| 646 | TGTGCGAGCC | SEQ ID NO: 748 | CCTGTCAGTT | SEQ ID NO: 1724 |
| 647 | CCAGATCTTC | SEQ ID NO: 749 | TAAGAGCATC | SEQ ID NO: 1725 |
| 648 | GGAAGGCGCC | SEQ ID NO: 750 | CAACGACAAG | SEQ ID NO: 1726 |
| 649 | TGTCTAGGAG | SEQ ID NO: 751 | GACCGCAGAA | SEQ ID NO: 1727 |
| 650 | GTGCCGAGGT | SEQ ID NO: 752 | GATCAACTCA | SEQ ID NO: 1728 |
| 651 | TAGGTCCGAG | SEQ ID NO: 753 | AAGGTCATTA | SEQ ID NO: 1729 |
| 652 | CTGATTAATG | SEQ ID NO: 754 | TTCCGGCGGT | SEQ ID NO: 1730 |
| 653 | GTTAGACGTG | SEQ ID NO: 755 | GTTCGTTAGG | SEQ ID NO: 1731 |
| 654 | CTTCGTCTCT | SEQ ID NO: 756 | ATTCCTGCTC | SEQ ID NO: 1732 |
| 653 | TTATAAGGCC | SEQ ID NO: 757 | GTGACGAACG | SEQ ID NO: 1733 |
| 656 | ATATCGTGAC | SEQ ID NO: 758 | CTAATGAGCA | SEQ ID NO: 1734 |
| 657 | ATCTTGGAGC | SEQ ID NO: 759 | ATGGTGAAGG | SEQ ID NO: 1735 |
| 658 | GAGGTAATTG | SEQ ID NO: 760 | GAACTCCTCG | SEQ ID NO: 1736 |
| 659 | TATTGTTGCA | SEQ ID NO: 761 | AGTTCATCTA | SEQ ID NO: 1737 |
| 660 | CCTATTGTCG | SEQ ID NO: 762 | TTGTCCAACT | SEQ ID NO: 1738 |
| 661 | ACATCTGCTA | SEQ ID NO: 763 | GCCGCTAACG | SEQ ID NO: 1739 |
| 662 | AAGTACCGTG | SEQ ID NO: 764 | TGACGTCCAG | SEQ ID NO: 1740 |
| 663 | AGGCGGTCAC | SEQ ID NO: 765 | GAGATCAGTC | SEQ ID NO: 1741 |
| 664 | AGGATGGTGC | SEQ ID NO: 766 | ACCGCCAGGA | SEQ ID NO: 1742 |
| 665 | GCAGGCCGTT | SEQ ID NO: 767 | GGTAGTTAGT | SEQ ID NO: 1743 |
| 666 | GTTCGTGGCG | SEQ ID NO: 768 | TGCGTTGATT | SEQ ID NO: 1744 |
| 667 | GCAATTGTTG | SEQ ID NO: 769 | GTGGTCGCCT | SEQ ID NO: 1745 |
| 668 | AAGTGGATGG | SEQ ID NO: 770 | AATGACTAGT | SEQ ID NO: 1746 |
| 669 | CTCCTCGTCT | SEQ ID NO: 771 | TCTTCGCACC | SEQ ID NO: 1747 |
| 670 | AATCCGAGTC | SEQ ID NO: 772 | AAGTCCATCT | SEQ ID NO: 1748 |
| 671 | ATCTTATGAA | SEQ ID NO: 773 | ATGAGCGACG | SEQ ID NO: 1749 |
| 672 | TACTGGAGCT | SEQ ID NO: 774 | CCGGTACCAC | SEQ ID NO: 1750 |
| 673 | AAGAGGACAC | SEQ ID NO: 775 | GCGCATAATG | SEQ ID NO: 1751 |
| 674 | CTTCACAGGT | SEQ ID NO: 776 | GCATTAGGTC | SEQ ID NO: 1752 |
| 675 | TCGGAATGCT | SEQ ID NO: 777 | TATCATCTTA | SEQ ID NO: 1753 |
| 676 | GACGTGGATT | SEQ ID NO: 778 | TTCGACGTTA | SEQ ID NO: 1754 |
| 677 | AGAGGTGGTG | SEQ ID NO: 779 | GAGACAGAGA | SEQ ID NO: 1755 |
| 678 | CGCTACACAC | SEQ ID NO: 780 | TCGCTACATA | SEQ ID NO: 1756 |
| 679 | GTTCTAGTCT | SEQ ID NO: 781 | CCAATGCTAT | SEQ ID NO: 1757 |
| 680 | ACAGGCTCTT | SEQ ID NO: 782 | GTAACGCTCA | SEQ ID NO: 1758 |
| 681 | CTCTCCTATA | SEQ ID NO: 783 | ATCCACACTC | SEQ ID NO: 1759 |
| 682 | AGGTATAGAT | SEQ ID NO: 784 | CTTAACCAGG | SEQ ID NO: 1760 |
| 683 | CTTCTCTGCG | SEQ ID NO: 785 | TACTAAGCTA | SEQ ID NO: 1761 |
| 684 | TCTGTCTTGC | SEQ ID NO: 786 | GTCCTCTAGT | SEQ ID NO: 1762 |
| 685 | GTGATGGTCG | SEQ ID NO: 787 | CGATATGTAT | SEQ ID NO: 1763 |
| 686 | CTGGATCTCA | SEQ ID NO: 788 | CATTAGCTAT | SEQ ID NO: 1764 |
| 687 | GCTATTCTAC | SEQ ID NO: 789 | GCCGTATGAT | SEQ ID NO: 1765 |
| 688 | TCCTCAGCTG | SEQ ID NO: 790 | AGCAAGGCCT | SEQ ID NO: 1766 |
| 689 | ATAAGGCAGG | SEQ ID NO: 791 | GACCATTGAA | SEQ ID NO: 1767 |
| 690 | ATAAGTCGTT | SEQ ID NO: 792 | GTCACGTAGC | SEQ ID NO: 1768 |
| 691 | TCGTTATACT | SEQ ID NO: 793 | CGTTATCACC | SEQ ID NO: 1769 |
| 692 | TTGGTCTTAT | SEQ ID NO: 794 | TCACTTGGCT | SEQ ID NO: 1770 |
| 693 | AAGGTCTGAT | SEQ ID NO: 795 | GTATTCTACT | SEQ ID NO: 1771 |
| 694 | GACATCTGCC | SEQ ID NO: 796 | GATGCATAAT | SEQ ID NO: 1772 |
| 695 | AGGCTCACTT | SEQ ID NO: 797 | GTGGCATCAG | SEQ ID NO: 1773 |
| 696 | CTATTCACAT | SEQ ID NO: 798 | ATGCGCCTCA | SEQ ID NO: 1774 |
| 697 | AGCACTATGT | SEQ ID NO: 799 | CGATGTCAAT | SEQ ID NO: 1775 |
| 698 | CGGCTACCGA | SEQ ID NO: 800 | ATAACATGGA | SEQ ID NO: 1776 |
| 699 | GCCGTGTAGT | SEQ ID NO: 801 | TGCATTAACG | SEQ ID NO: 1777 |
| 700 | GCGTCAAGAG | SEQ ID NO: 802 | TACCACTACA | SEQ ID NO: 1778 |
| 701 | GAGGAAGACC | SEQ ID NO: 803 | CAACATTAGG | SEQ ID NO: 1779 |
| 702 | ACGTCTGTTG | SEQ ID NO: 804 | GTCCTTGACT | SEQ ID NO: 1780 |
| 703 | AGGCGATAGG | SEQ ID NO: 805 | GTGCTACTGA | SEQ ID NO: 1781 |
| 704 | TGTTGTCGTA | SEQ ID NO: 806 | TCAGGCAGCC | SEQ ID NO: 1782 |
| 705 | ACCTAGGCAC | SEQ ID NO: 807 | CAGGCGATGA | SEQ ID NO: 1783 |
| 706 | CGTCTTCAGG | SEQ ID NO: 808 | TTAGTAGGTT | SEQ ID NO: 1784 |
| 707 | AGGCTTCAAT | SEQ ID NO: 809 | GAACGACGGC | SEQ ID NO: 1785 |
| 708 | ACTATGCTCC | SEQ ID NO: 810 | AACGCTCTAG | SEQ ID NO: 1786 |
| 709 | GTCATCTTAG | SEQ ID NO: 811 | CGCGCCATCT | SEQ ID NO: 1787 |
| 710 | CTCGATGTGT | SEQ ID NO: 812 | CAGTCCTACT | SEQ ID NO: 1788 |
| 711 | AGAGCGGCTT | SEQ ID NO: 813 | CGGAACGCAA | SEQ ID NO: 1789 |
| 712 | GCGGATGTGA | SEQ ID NO: 814 | GGAGTGATGT | SEQ ID NO: 1790 |
| 713 | CTATACGGAC | SEQ ID NO: 815 | TGCTAGGATC | SEQ ID NO: 1791 |
| 714 | CTGTCAGACT | SEQ ID NO: 816 | TACGCTAGCT | SEQ ID NO: 1792 |
| 715 | GAAGAGGTGC | SEQ ID NO: 817 | GGCGACGCTG | SEQ ID NO: 1793 |
| 716 | GACCTATGTA | SEQ ID NO: 818 | CCGCGCACTT | SEQ ID NO: 1794 |
| 717 | GAATAAGGCT | SEQ ID NO: 819 | CAGGATAGAT | SEQ ID NO: 1795 |
| 718 | GAGGCATGCA | SEQ ID NO: 820 | GTAGCTTAGA | SEQ ID NO: 1796 |
| 719 | CCATGAGGAC | SEQ ID NO: 821 | GGAGAGCCGA | SEQ ID NO: 1797 |
| 720 | GAGTAGTCTG | SEQ ID NO: 822 | GATAATGCGA | SEQ ID NO: 1798 |
| 721 | CTGTGAGAGG | SEQ ID NO: 823 | GACCAGTAAT | SEQ ID NO: 1799 |
| 722 | GTTGGATATA | SEQ ID NO: 824 | TGGCATCTGG | SEQ ID NO: 1800 |
| 723 | AGTGCGAGTA | SEQ ID NO: 825 | ATAATATTGG | SEQ ID NO: 1801 |
| 724 | CGTGGACAAT | SEQ ID NO: 826 | CTAGCAGACA | SEQ ID NO: 1802 |
| 725 | ATCCGTATAC | SEQ ID NO: 827 | ATTACAGTGC | SEQ ID NO: 1803 |
| 726 | TACTGCGTGA | SEQ ID NO: 828 | ACTCGGCGTG | SEQ ID NO: 1804 |
| 727 | CGTCATCGAC | SEQ ID NO: 829 | TACGTTAGGC | SEQ ID NO: 1805 |
| 728 | CTGTCTACCT | SEQ ID NO: 830 | TATAAGTCCG | SEQ ID NO: 1806 |
| 729 | GGAGGACTAG | SEQ ID NO: 831 | GGAATTACGG | SEQ ID NO: 1807 |
| 730 | CAAGGCCTCA | SEQ ID NO: 832 | GACGATACAT | SEQ ID NO: 1808 |
| 731 | GAGGTATGTT | SEQ ID NO: 833 | GATAGCCAAG | SEQ ID NO: 1809 |
| 732 | TGGTACATAC | SEQ ID NO: 834 | TGAGCTCTGC | SEQ ID NO: 1810 |
| 733 | CTTCGAACAT | SEQ ID NO: 835 | TGCGCAGAAT | SEQ ID NO: 1811 |
| 734 | TCTTGACTGT | SEQ ID NO: 836 | GCCTTCCTGC | SEQ ID NO: 1812 |
| 735 | AAGTGATGCG | SEQ ID NO: 837 | GAGCGACCTG | SEQ ID NO: 1813 |
| 736 | ACACACAGGC | SEQ ID NO: 838 | GCAGACGCCA | SEQ ID NO: 1814 |
| 737 | ACTTCGGAGG | SEQ ID NO: 839 | CGACTAGGTA | SEQ ID NO: 1815 |
| 738 | GATGGACGTT | SEQ ID NO: 840 | CAATCTGTGC | SEQ ID NO: 1816 |
| 739 | CGGTTGTCTT | SEQ ID NO: 841 | GTCCATTACG | SEQ ID NO: 1817 |
| 740 | TCTCCGATGG | SEQ ID NO: 842 | AGATTGAAGT | SEQ ID NO: 1818 |
| 741 | ACAAGGCTTA | SEQ ID NO: 843 | GTTCAACGAC | SEQ ID NO: 1819 |
| 742 | TGCATCTCGT | SEQ ID NO: 844 | CGTAATGTCC | SEQ ID NO: 1820 |
| 743 | CGAGTTGGAT | SEQ ID NO: 845 | GCCAGTACGG | SEQ ID NO: 1821 |
| 744 | TCTGGCTATT | SEQ ID NO: 846 | CATTGTCTTA | SEQ ID NO: 1822 |
| 745 | CTGTATTAAG | SEQ ID NO: 847 | CGTAGATCGC | SEQ ID NO: 1823 |
| 746 | CGTGCGCATC | SEQ ID NO: 848 | CTTAGCCTCC | SEQ ID NO: 1824 |
| 747 | TGAGGCTTAG | SEQ ID NO: 849 | GAACCACAGG | SEQ ID NO: 1825 |
| 748 | AGCAGGAGGC | SEQ ID NO: 850 | AAGGTATATC | SEQ ID NO: 1826 |
| 749 | GCGATATGTA | SEQ ID NO: 851 | GTATGCAATG | SEQ ID NO: 1827 |
| 750 | CGTGAAGTTC | SEQ ID NO: 852 | TGCAACCGTG | SEQ ID NO: 1828 |
| 751 | CAAGCGTCAG | SEQ ID NO: 853 | ACACCGTCGG | SEQ ID NO: 1829 |
| 752 | AGGCGGATGC | SEQ ID NO: 854 | GGCCAAGTGA | SEQ ID NO: 1830 |
| 753 | ATACAGCGTT | SEQ ID NO: 855 | CAAGACTCTC | SEQ ID NO: 1831 |
| 754 | CCATGGCTCA | SEQ ID NO: 856 | GAAGTAGCAT | SEQ ID NO: 1832 |
| 755 | GTAGGCTCAG | SEQ ID NO: 857 | GCAGGCAAGG | SEQ ID NO: 1833 |
| 756 | CTAGTGTCTT | SEQ ID NO: 858 | TCTGGTCAAC | SEQ ID NO: 1834 |
| 757 | GACGTCTCAC | SEQ ID NO: 859 | GCGTAACACA | SEQ ID NO: 1835 |
| 758 | ACACATACAG | SEQ ID NO: 860 | AATATCAGCA | SEQ ID NO: 1836 |
| 759 | ATAGGCAATA | SEQ ID NO: 861 | ACAGGATACC | SEQ ID NO: 1837 |
| 760 | GTAGAGCGCG | SEQ ID NO: 862 | CTAATGCATA | SEQ ID NO: 1838 |
| 761 | GGTATACAGC | SEQ ID NO: 863 | TGTGTAACTG | SEQ ID NO: 1839 |
| 762 | AGTCTAGTTC | SEQ ID NO: 864 | CCTGTGATAC | SEQ ID NO: 1840 |
| 763 | CTACAAGCGT | SEQ ID NO: 865 | AACGTCCAGT | SEQ ID NO: 1841 |
| 764 | CTGAGGTGCG | SEQ ID NO: 866 | GGAGCTACCG | SEQ ID NO: 1842 |
| 765 | CGTGAATCTT | SEQ ID NO: 867 | AGTATCGTAC | SEQ ID NO: 1843 |
| 766 | CGTCGACTAG | SEQ ID NO: 868 | TTGGTCGTTG | SEQ ID NO: 1844 |
| 767 | ATTAAGCGTG | SEQ ID NO: 869 | GTCACGACAT | SEQ ID NO: 1845 |
| 768 | TCCGGCGTCG | SEQ ID NO: 870 | AATGCATCGT | SEQ ID NO: 1846 |
| 769 | AGGAGGCCAG | SEQ ID NO: 871 | AGGACATAAC | SEQ ID NO: 1847 |
| 770 | GGATGGTGCA | SEQ ID NO: 872 | CGGTCATGTG | SEQ ID NO: 1848 |
| 771 | CTGGCGGAAG | SEQ ID NO: 873 | CGACTTATCT | SEQ ID NO: 1849 |
| 772 | TCAGTTGCAA | SEQ ID NO: 874 | ACCACGAGCC | SEQ ID NO: 1850 |
| 773 | GTCTTATTGG | SEQ ID NO: 875 | GGCTGAACGG | SEQ ID NO: 1851 |
| 774 | GCCTAAGAGG | SEQ ID NO: 876 | GCCAGGCGAA | SEQ ID NO: 1852 |
| 775 | AGTCTAAGGA | SEQ ID NO: 877 | GAATGCGGTC | SEQ ID NO: 1853 |
| 776 | GAGTCTGTGA | SEQ ID NO: 878 | TCTAACAACG | SEQ ID NO: 1854 |
| 777 | CTACATCGTC | SEQ ID NO: 879 | TTATACCGAA | SEQ ID NO: 1855 |
| 778 | TATATCTCAG | SEQ ID NO: 880 | ACACCACAGT | SEQ ID NO: 1856 |
| 779 | CCGTCACGTT | SEQ ID NO: 881 | TCAGACACCG | SEQ ID NO: 1857 |
| 780 | TATCGAGGCC | SEQ ID NO: 882 | GTAGCCACAA | SEQ ID NO: 1858 |
| 781 | TGAGGTATCT | SEQ ID NO: 883 | GACGAGGCGA | SEQ ID NO: 1859 |
| 782 | ATCGTTGAAT | SEQ ID NO: 884 | ATCTACATAT | SEQ ID NO: 1860 |
| 783 | CGTGCATGTA | SEQ ID NO: 885 | TGAGACGTTG | SEQ ID NO: 1861 |
| 784 | CGGACACCTT | SEQ ID NO: 886 | ATTCTGCCGA | SEQ ID NO: 1862 |
| 785 | AGTGGAGTCC | SEQ ID NO: 887 | CAGATCGAGA | SEQ ID NO: 1863 |
| 786 | TTGTGCATGC | SEQ ID NO: 888 | GAGCGCTGTT | SEQ ID NO: 1864 |
| 787 | TCTAAGGCAT | SEQ ID NO: 889 | GCACAATTAT | SEQ ID NO: 1865 |
| 788 | ATGAGGTATC | SEQ ID NO: 890 | GCAATTCGCC | SEQ ID NO: 1866 |
| 789 | CGGCTGTGAT | SEQ ID NO: 891 | ATATATAGTA | SEQ ID NO: 1867 |
| 790 | CCACGTGCGA | SEQ ID NO: 892 | AACCGTAGTT | SEQ ID NO: 1868 |
| 791 | GGCATGGAGT | SEQ ID NO: 893 | CACATTGTCA | SEQ ID NO: 1869 |
| 792 | CGATGTCGTG | SEQ ID NO: 894 | AGACAGTCAA | SEQ ID NO: 1870 |
| 793 | GAAGGCTGCG | SEQ ID NO: 895 | TGACAAGGAC | SEQ ID NO: 1871 |
| 794 | GCGTTATGCG | SEQ ID NO: 896 | TATATAGCCG | SEQ ID NO: 1872 |
| 795 | CACACATGCG | SEQ ID NO: 897 | GTTCTCAGAT | SEQ ID NO: 1873 |
| 796 | GCCTCGAAGG | SEQ ID NO: 898 | GATAATCTCC | SEQ ID NO: 1874 |
| 797 | CCGGCAGGTC | SEQ ID NO: 899 | GGTCCTTGTA | SEQ ID NO: 1875 |
| 798 | CGTGAAGGCA | SEQ ID NO: 900 | GAACAGACTG | SEQ ID NO: 1876 |
| 799 | GCGACATCGT | SEQ ID NO: 901 | GAAGAATCTA | SEQ ID NO: 1877 |
| 800 | CGTCGCGATG | SEQ ID NO: 902 | CGTTGAATTG | SEQ ID NO: 1878 |
| 801 | GAGGCTGAGC | SEQ ID NO: 903 | GGTACCGCTG | SEQ ID NO: 1879 |
| 802 | AGGCTGGCCT | SEQ ID NO: 904 | GTGCACGCAG | SEQ ID NO: 1880 |
| 803 | TGGTGTTATA | SEQ ID NO: 905 | ATTCGATATT | SEQ ID NO: 1881 |
| 804 | CGTGCGTGCG | SEQ ID NO: 906 | CTGAATGACC | SEQ ID NO: 1882 |
| 805 | CGAGGTGACG | SEQ ID NO: 907 | CTATTAAGGA | SEQ ID NO: 1883 |
| 806 | GTGTTAGGCT | SEQ ID NO: 908 | GAATCACAAT | SEQ ID NO: 1884 |
| 807 | CGAGGCACAG | SEQ ID NO: 909 | AAGGACCTCT | SEQ ID NO: 1885 |
| 808 | CGCGTCTCAG | SEQ ID NO: 910 | TCTCAATACA | SEQ ID NO: 1886 |
| 809 | TATAGCTGTG | SEQ ID NO: 911 | ATGAAGCCAT | SEQ ID NO: 1887 |
| 810 | CTTAGTACTC | SEQ ID NO: 912 | CCAATCTACC | SEQ ID NO: 1888 |
| 811 | ATCGTCTCTC | SEQ ID NO: 913 | TCTGAAGTCC | SEQ ID NO: 1889 |
| 812 | TTCAGGCTTA | SEQ ID NO: 914 | GCAAGGTTCA | SEQ ID NO: 1890 |
| 813 | TCGTGTCACG | SEQ ID NO: 915 | CGTAATCAAG | SEQ ID NO: 1891 |
| 814 | CTTAACGGAA | SEQ ID NO: 916 | TGTGAATATA | SEQ ID NO: 1892 |
| 815 | GAGGCGTGGC | SEQ ID NO: 917 | GGTTGAGTAA | SEQ ID NO: 1893 |
| 816 | TATAGCGTAG | SEQ ID NO: 918 | ACGTAGACCA | SEQ ID NO: 1894 |
| 817 | TGCAAGTCAG | SEQ ID NO: 919 | TATCGACAGA | SEQ ID NO: 1895 |
| 818 | CGTGCCGCAT | SEQ ID NO: 920 | ATCGTACTGT | SEQ ID NO: 1896 |
| 819 | GTGAGTACGT | SEQ ID NO: 921 | TAAGGCTTGT | SEQ ID NO: 1897 |
| 820 | TTACGTAAGC | SEQ ID NO: 922 | TGTAGCCTGA | SEQ ID NO: 1898 |
| 821 | GGAGTCGAGG | SEQ ID NO: 923 | GGACCATAGC | SEQ ID NO: 1899 |
| 822 | ATGGCGTCTC | SEQ ID NO: 924 | CGGTGGCAGA | SEQ ID NO: 1900 |
| 823 | CGATCTCCGT | SEQ ID NO: 925 | ACTCCGGTCA | SEQ ID NO: 1901 |
| 824 | ACGAATTATA | SEQ ID NO: 926 | GTTACTGGTG | SEQ ID NO: 1902 |
| 825 | AGGCTCGGTC | SEQ ID NO: 927 | GGATCGCGGC | SEQ ID NO: 1903 |
| 826 | ATGCAGTCGA | SEQ ID NO: 928 | AGATGGTAAC | SEQ ID NO: 1904 |
| 827 | ATCTCGTATC | SEQ ID NO: 929 | GCTGAACCAC | SEQ ID NO: 1905 |
| 828 | AATCTTATGG | SEQ ID NO: 930 | GCAGGCTTCC | SEQ ID NO: 1906 |
| 829 | CGAACTTGAT | SEQ ID NO: 931 | AACGCTACGA | SEQ ID NO: 1907 |
| 830 | AGGTGCGTCG | SEQ ID NO: 932 | GTCATGCAGG | SEQ ID NO: 1908 |
| 831 | TTATACTACA | SEQ ID NO: 933 | CTCTCTATCC | SEQ ID NO: 1909 |
| 832 | GCAACGCGTT | SEQ ID NO: 934 | TAAGTTAGAT | SEQ ID NO: 1910 |
| 833 | CATGGTGTGT | SEQ ID NO: 935 | AACAATACAA | SEQ ID NO: 1911 |
| 834 | CTGTGGATAA | SEQ ID NO: 936 | TGTTAGGCTG | SEQ ID NO: 1912 |
| 835 | TTGGAAGTTC | SEQ ID NO: 937 | ACCTCGATGT | SEQ ID NO: 1913 |
| 836 | AGTACTAATG | SEQ ID NO: 938 | ATGTATCGAA | SEQ ID NO: 1914 |
| 837 | AGAAGAGGAC | SEQ ID NO: 939 | GCATCACTTG | SEQ ID NO: 1915 |
| 838 | GTTGATTGTA | SEQ ID NO: 940 | CCGATGACTT | SEQ ID NO: 1916 |
| 839 | GCGAGCGTTG | SEQ ID NO: 941 | TTATGACCTC | SEQ ID NO: 1917 |
| 840 | TTCGGAAGGA | SEQ ID NO: 942 | GAATAACGAC | SEQ ID NO: 1918 |
| 841 | TGATCGGAGC | SEQ ID NO: 943 | CATGTTGCAT | SEQ ID NO: 1919 |
| 842 | CTCGAGACTT | SEQ ID NO: 944 | CAATCCTTCC | SEQ ID NO: 1920 |
| 843 | TCAATCGATT | SEQ ID NO: 945 | TAGGCCACGC | SEQ ID NO: 1921 |
| 844 | AAGAGCGCTA | SEQ ID NO: 946 | AGATGACACC | SEQ ID NO: 1922 |
| 845 | CATGAGTGAG | SEQ ID NO: 947 | AATCGAACAG | SEQ ID NO: 1923 |
| 846 | TCACGCGCGT | SEQ ID NO: 948 | ACCGTATCAG | SEQ ID NO: 1924 |
| 847 | GTTGTGAGCT | SEQ ID NO: 949 | TGGTAGTTGC | SEQ ID NO: 1925 |
| 848 | GCTAGCGAGG | SEQ ID NO: 950 | GGAGTTCGAG | SEQ ID NO: 1926 |
| 849 | GCGCAGCGAG | SEQ ID NO: 951 | CCTACTAAGA | SEQ ID NO: 1927 |
| 850 | CTATGAGTCA | SEQ ID NO: 952 | ATCGAGAATA | SEQ ID NO: 1928 |
| 851 | CCGTGCATCA | SEQ ID NO: 953 | AACCTACACG | SEQ ID NO: 1929 |
| 852 | AATTAGTGTC | SEQ ID NO: 954 | ATAAGCTGCA | SEQ ID NO: 1930 |
| 853 | CGGACTGTGC | SEQ ID NO: 955 | ATGACTCCGG | SEQ ID NO: 1931 |
| 854 | CGTGTTACGG | SEQ ID NO: 956 | CTCTGGACAC | SEQ ID NO: 1932 |
| 855 | TACAAGGCTG | SEQ ID NO: 957 | GCGCCAACTG | SEQ ID NO: 1933 |
| 856 | GTATTAATAG | SEQ ID NO: 958 | CACACGGCCG | SEQ ID NO: 1934 |
| 857 | GCCTCGGATA | SEQ ID NO: 959 | GTCACACAAT | SEQ ID NO: 1935 |
| 858 | GACGTCCGAA | SEQ ID NO: 960 | CGTCGCAAGC | SEQ ID NO: 1936 |
| 859 | GTTATGATAT | SEQ ID NO: 961 | CGGTAGCAAT | SEQ ID NO: 1937 |
| 860 | TAGGCGTCTA | SEQ ID NO: 962 | GTCTTACCTC | SEQ ID NO: 1938 |
| 861 | CCTATATAGC | SEQ ID NO: 963 | AACAAGCACT | SEQ ID NO: 1939 |
| 862 | TTGAATTCAC | SEQ ID NO: 964 | CTTAGCGAGT | SEQ ID NO: 1940 |
| 863 | GCTCTCTATA | SEQ ID NO: 965 | GAGGTGTTCA | SEQ ID NO: 1941 |
| 864 | ATTCATCTCC | SEQ ID NO: 966 | ATTATGCATC | SEQ ID NO: 1942 |
| 865 | ATGGAAGCGG | SEQ ID NO: 967 | GCGACGGATC | SEQ ID NO: 1943 |
| 366 | CAGGTAGCTA | SEQ ID NO: 968 | CAAGCAGGTA | SEQ ID NO: 1944 |
| 867 | CCGTGAATTC | SEQ ID NO: 969 | CCACACGTAG | SEQ ID NO: 1945 |
| 868 | CGTGTCGGTG | SEQ ID NO: 970 | TCAGTCGCGG | SEQ ID NO: 1946 |
| 869 | CCGTCGAGTG | SEQ ID NO: 971 | ATGATCGCTC | SEQ ID NO: 1947 |
| 870 | AGGACGTCGT | SEQ ID NO: 972 | AGGCGTAACT | SEQ ID NO: 1948 |
| 871 | GCAGAGTGTC | SEQ ID NO: 973 | TATGAACACA | SEQ ID NO: 1949 |
| 872 | TTCCACGTGG | SEQ ID NO: 974 | ACAATCGTAG | SEQ ID NO: 1950 |
| 873 | TGGAGGCTCC | SEQ ID NO: 975 | ATAGAGGACA | SEQ ID NO: 1951 |
| 874 | TGGAGATCGG | SEQ ID NO: 976 | AGTGTACATG | SEQ ID NO: 1952 |
| 875 | ATCTTACGTG | SEQ ID NO: 977 | GCGTGACATC | SEQ ID NO: 1953 |
| 876 | TAGGTGACGT | SEQ ID NO: 978 | ACCACAGCAA | SEQ ID NO: 1954 |
| 877 | GTCTCCTTAT | SEQ ID NO: 979 | TCAGTTAACC | SEQ ID NO: 1955 |
| 878 | TTGAGAGGCT | SEQ ID NO: 980 | AGGACTTAGA | SEQ ID NO: 1956 |
| 879 | GTGTGTGTCA | SEQ ID NO: 981 | CTGAGTATCT | SEQ ID NO: 1957 |
| 880 | TCTAGAACTT | SEQ ID NO: 982 | CGGCCTATAT | SEQ ID NO: 1958 |
| 881 | GCGTGTCCTG | SEQ ID NO: 983 | GCGTAGTGAT | SEQ ID NO: 1959 |
| 882 | GGATCCAATC | SEQ ID NO: 984 | CGGCGAGCGG | SEQ ID NO: 1960 |
| 883 | GACCGATCGG | SEQ ID NO: 985 | CAGTGTGGCT | SEQ ID NO: 1961 |
| 884 | TGGCGTAGGT | SEQ ID NO: 986 | ATGAATAGGT | SEQ ID NO: 1962 |
| 885 | GAAGACGCGT | SEQ ID NO: 987 | TGGTCCTCGA | SEQ ID NO: 1963 |
| 886 | CGAGCGTGAC | SEQ ID NO: 988 | ACGTGCGGTT | SEQ ID NO: 1964 |
| 887 | GCATGCCATA | SEQ ID NO: 989 | CGTGTTCACA | SEQ ID NO: 1965 |
| 888 | CCGCTGCGTC | SEQ ID NO: 990 | GTTAATCGTC | SEQ ID NO: 1966 |
| 889 | CCATTAATGC | SEQ ID NO: 991 | ATGTCACAGT | SEQ ID NO: 1967 |
| 890 | GGCATGCCTA | SEQ ID NO: 992 | CTGGCTACTG | SEQ ID NO: 1968 |
| 891 | ACGCGTCGTT | SEQ ID NO: 993 | CATCTGGTCA | SEQ ID NO: 1969 |
| 892 | GACAACGTTG | SEQ ID NO: 994 | TTACGCTCTA | SEQ ID NO: 1970 |
| 893 | GTCATATATG | SEQ ID NO: 995 | TCGATTCATT | SEQ ID NO: 1971 |
| 894 | CCGTCGTACC | SEQ ID NO: 996 | ATGAAGATCA | SEQ ID NO: 1972 |
| 895 | ATGTGTTGGA | SEQ ID NO: 997 | CCATCTAAGT | SEQ ID NO: 1973 |
| 896 | AATGGCCATG | SEQ ID NO: 998 | GCGAACAACT | SEQ ID NO: 1974 |
| 897 | CTACTCGAGT | SEQ ID NO: 999 | CACACACCTC | SEQ ID NO: 1975 |
| 898 | AAGAGCGGAT | SEQ ID NO: 1000 | GCCGACACCT | SEQ ID NO: 1976 |
| 899 | CGGTCGTGGA | SEQ ID NO: 1001 | TCGTATGAGC | SEQ ID NO: 1977 |
| 900 | ATGTAGGTAC | SEQ ID NO: 1002 | GGTTACGAGA | SEQ ID NO: 1978 |
| 901 | AGCGCGTACG | SEQ ID NO: 1003 | GATCAGAGCC | SEQ ID NO: 1979 |
| 902 | TAGCTATGCC | SEQ ID NO: 1004 | AGAGCCTGTC | SEQ ID NO: 1980 |
| 903 | CTGTTCTATG | SEQ ID NO: 1005 | GAGCTAGCCT | SEQ ID NO: 1981 |
| 904 | AAGTGCGAGG | SEQ ID NO: 1006 | CAGAGGTTCC | SEQ ID NO: 1982 |
| 905 | CTTAAGCTAG | SEQ ID NO: 1007 | TCTGAGACCT | SEQ ID NO: 1983 |
| 906 | GAGGTTATGA | SEQ ID NO: 1008 | AGTCTCTAGG | SEQ ID NO: 1984 |
| 907 | CGTCGTGAAC | SEQ ID NO: 1009 | AGTCCACGTA | SEQ ID NO: 1985 |
| 908 | TATCAATTGA | SEQ ID NO: 1010 | ACTTCTAGAG | SEQ ID NO: 1986 |
| 909 | GTACAGGATA | SEQ ID NO: 1011 | GGCTTCTGAT | SEQ ID NO: 1987 |
| 910 | GGAGATGCAT | SEQ ID NO: 1012 | CCATGGTGGC | SEQ ID NO: 1988 |
| 911 | CCTGCTAGCA | SEQ ID NO: 1013 | AGAGCTTGCG | SEQ ID NO: 1989 |
| 912 | GATGGTTGGC | SEQ ID NO: 1014 | TCTTCCGAAT | SEQ ID NO: 1990 |
| 913 | TAGACCGGTC | SEQ ID NO: 1015 | GGTTGCCGCA | SEQ ID NO: 1991 |
| 914 | GGCGTACGTA | SEQ ID NO: 1016 | GCACAAGTGG | SEQ ID NO: 1992 |
| 915 | CGGTGGAGGT | SEQ ID NO: 1017 | GACTTCTTCA | SEQ ID NO: 1993 |
| 916 | CCGATTCGAT | SEQ ID NO: 1018 | TAAGACAGAC | SEQ ID NO: 1994 |
| 917 | CGAGTGCTAG | SEQ ID NO: 1019 | TGGTGACCAC | SEQ ID NO: 1995 |
| 918 | AGGAGTTGCG | SEQ ID NO: 1020 | GACTAATAAG | SEQ ID NO: 1996 |
| 919 | ATATGAGCGT | SEQ ID NO: 1021 | GCAACCGTTC | SEQ ID NO: 1997 |
| 920 | GTCTCGCGTA | SEQ ID NO: 1022 | TTGAACGGCA | SEQ ID NO: 1998 |
| 921 | CGGAGTCCGG | SEQ ID NO: 1023 | ATGGCCACCT | SEQ ID NO: 1999 |
| 922 | CATGGAGGAC | SEQ ID NO: 1024 | AAGAGGAATG | SEQ ID NO: 2000 |
| 923 | AAGGCTAACG | SEQ ID NO: 1025 | GCAGGTGGAA | SEQ ID NO: 2001 |
| 924 | AACGTGTGGT | SEQ ID NO: 1026 | CGCCGAATAT | SEQ ID NO: 2002 |
| 925 | GTGCCGTGTG | SEQ ID NO: 1027 | CAACGTGCCG | SEQ ID NO: 2003 |
| 926 | CGCCTAGGCC | SEQ ID NO: 1028 | ACAGGTACAC | SEQ ID NO: 2004 |
| 927 | TCGTGTGGAT | SEQ ID NO: 1029 | GAACGTAAGG | SEQ ID NO: 2005 |
| 928 | CCGCGGCTAT | SEQ ID NO: 1030 | GCCTAACAAT | SEQ ID NO: 2006 |
| 929 | TTGTCGTGTA | SEQ ID NO: 1031 | AACGTGCGCG | SEQ ID NO: 2007 |
| 930 | CTTGCTGTCT | SEQ ID NO: 1032 | AGGTACGGCT | SEQ ID NO: 2008 |
| 931 | TAGCGTGTCT | SEQ ID NO: 1033 | TACCAACGTA | SEQ ID NO: 2009 |
| 932 | TATACGCTCT | SEQ ID NO: 1034 | CTAAGCAAGA | SEQ ID NO: 2010 |
| 933 | CAAGAGGCTA | SEQ ID NO: 1035 | CTCGCAGGAC | SEQ ID NO: 2011 |
| 934 | TTCGATATCG | SEQ ID NO: 1036 | ATCGTCGTCC | SEQ ID NO: 2012 |
| 935 | ATGTCTCTAC | SEQ ID NO: 1037 | TCACCGCTCC | SEQ ID NO: 2013 |
| 936 | CCGGCTTGGC | SEQ ID NO: 1038 | TTATATTCAT | SEQ ID NO: 2014 |
| 937 | CCGATCGCGG | SEQ ID NO: 1039 | CATTGTGATT | SEQ ID NO: 2015 |
| 938 | CACTAGTGCG | SEQ ID NO: 1040 | AAGGCTGGTT | SEQ ID NO: 2016 |
| 939 | CGTGTCTTCC | SEQ ID NO: 1041 | AGGAGGATAT | SEQ ID NO: 2017 |
| 940 | CCGTATATAC | SEQ ID NO: 1042 | ACGACCGTCA | SEQ ID NO: 2018 |
| 941 | CCGTGTCTGA | SEQ ID NO: 1043 | CGCGTAGTGG | SEQ ID NO: 2019 |
| 942 | CCGGAGTCGC | SEQ ID NO: 1044 | ATTCACGCTG | SEQ ID NO: 2020 |
| 943 | CGGATCATCC | SEQ ID NO: 1045 | AGTGTTGCAC | SEQ ID NO: 2021 |
| 944 | CTATGTTACG | SEQ ID NO: 1046 | ACGATTGAGC | SEQ ID NO: 2022 |
| 945 | TATACCAGGA | SEQ ID NO: 1047 | GCAATCAATG | SEQ ID NO: 2023 |
| 946 | GATGAGGAGT | SEQ ID NO: 1048 | GGCATCCAAC | SEQ ID NO: 2024 |
| 947 | GTGTCTCCAT | SEQ ID NO: 1049 | TATGTCGCTC | SEQ ID NO: 2025 |
| 948 | GAGAGCGTCA | SEQ ID NO: 1050 | TGCGTTCGAC | SEQ ID NO: 2026 |
| 949 | ATGTTGAGCA | SEQ ID NO: 1051 | TTGAAGCGAG | SEQ ID NO: 2027 |
| 950 | TATACTCAAT | SEQ ID NO: 1052 | GCCTCACTGA | SEQ ID NO: 2028 |
| 951 | TCGGCTATGT | SEQ ID NO: 1053 | CTATAGCAAG | SEQ ID NO: 2029 |
| 952 | GTAGGCTAGC | SEQ ID NO: 1054 | GGTGCAACGG | SEQ ID NO: 2030 |
| 953 | GGAGCGTCGC | SEQ ID NO: 1055 | GGCCGCGTAG | SEQ ID NO: 2031 |
| 954 | ATGCGACCAC | SEQ ID NO: 1056 | AAGAGAGAGT | SEQ ID NO: 2032 |
| 955 | CCGAAGGAGG | SEQ ID NO: 1057 | AGGTTGTAGG | SEQ ID NO: 2033 |
| 956 | CTCCGAGGCG | SEQ ID NO: 1058 | TACTTAGGAA | SEQ ID NO: 2034 |
| 957 | GCTATGACGT | SEQ ID NO: 1059 | AAGGTCGTGG | SEQ ID NO: 2035 |
| 958 | GTCTATGTGG | SEQ ID NO: 1060 | TGGAGTTAAT | SEQ ID NO: 2036 |
| 959 | TATACAACCT | SEQ ID NO: 1061 | TAACCGCAAG | SEQ ID NO: 2037 |
| 960 | CCGAGAGTCG | SEQ ID NO: 1062 | ATTAGTCCTG | SEQ ID NO: 2038 |
| 961 | CTTATAGGAT | SEQ ID NO: 1063 | ATAGGTGGCA | SEQ ID NO: 2039 |
| 962 | CGGATATACA | SEQ ID NO: 1064 | GAGTGCCATG | SEQ ID NO: 2040 |
| 963 | GGCCAGAGTC | SEQ ID NO: 1065 | TTGAGAATCA | SEQ ID NO: 2041 |
| 964 | CGGATGCTGT | SEQ ID NO: 1066 | GGCTGGTCCG | SEQ ID NO: 2042 |
| 965 | CGAGATATAC | SEQ ID NO: 1067 | CGGCGCTCGC | SEQ ID NO: 2043 |
| 966 | GGATCCAGGT | SEQ ID NO: 1068 | GCAATAGAAC | SEQ ID NO: 2044 |
| 967 | GTAATTACAC | SEQ ID NO: 1069 | TCGCCTTGCG | SEQ ID NO: 2045 |
| 968 | CACGTGAGTA | SEQ ID NO: 1070 | CCTCTTCGTA | SEQ ID NO: 2046 |
| 969 | CCTTAAGGAA | SEQ ID NO: 1071 | GATGATATGG | SEQ ID NO: 2047 |
| 970 | AGATTATAAT | SEQ ID NO: 1072 | GAGCGGCTTA | SEQ ID NO: 2048 |
| 971 | AGTCTCTTAT | SEQ ID NO: 1073 | ATGTTAACAT | SEQ ID NO: 2049 |
| 972 | AAGGCTATGC | SEQ ID NO: 1074 | AAGGATCGCG | SEQ ID NO: 2050 |
| 973 | TAATATTAAG | SEQ ID NO: 1075 | ATGGCATGGT | SEQ ID NO: 2051 |
| 974 | TGCAAGATCC | SEQ ID NO: 1076 | CTAATAACCT | SEQ ID NO: 2052 |
| 975 | TGTCGATCGA | SEQ ID NO: 1077 | ACTCGCACAT | SEQ ID NO: 2053 |
| 976 | AGATCGGTTA | SEQ ID NO: 1078 | ATGATATATT | SEQ ID NO: 2054 |

Specific sequences of an I5 sequencing adapter and a Nextera I7 sequencing adapter are shown in Table 3.

TABLE 3
I5 sequencing adapter and Nextera I7 sequencing adapter

| Adapter name | Sequence | Sequence No. |
|---|---|---|
| I5 sequencing | AATGATACGGCGACCACCGAGATCTACA | SEQ ID NO: 98 |
| adapter | | |
| Nextera I7 | CTGTCTCTTATACACATCTCCGAGCCCACG | SEQ ID NO: 2069 |
| sequencing adapter | AGA | |

An example of primers for first synthesis of a DUDI and an Illumina sequencing adapter is as follows:

a forward primer:
(SEQ ID NO: 2065)
CACGACGCTCTTCCGATCTtcagtatcctCAAACATAGACTCCTCGCAT
AGCCT;
and
a reverse primer:
(SEQ ID NO: 2066)
CTCGGAGATGTGTATAAGAGACAGcacgccaacgACCTCCATCCGAGAC
ACACG.

2. An undiluted third PCR preamplification product was adopted as a PCR template. One tube of the PCR template was prepared for each sample.

3. A 30 μL PCR system was prepared from the following reagents: 2× PCR enzyme (including UDG and UTP): 15 μL, water: 12 μL, forward primer (10 μM) for adding a barcode and a sequencing adapter: 0.5 μL, reverse primer (10 μM) for adding the barcode and the sequencing adapter: 0.5 μL, and third PCR preamplification product: 2 μL.

4. qPCR for barcode addition

Each third PCR preamplification product was subjected to qPCR with a specific forward primer for adding a barcode and a sequencing adapter and a reverse primer for adding the barcode and the sequencing adapter, and a PCR procedure was as follows: 37° C. for 10 min; (1) 95° C. for 10 min; (2) 3 cycles of (95° C. for 15 s, 62° C. for 30 s, and 72° C. for 1 min); (3) 2 cycles of (95° C. for 15 s, 64° C. for 30 s, and 72° C. for 1 min); and (4) 45 cycles of (95° C. for 15 s, 68° C. 30 s, and 72° C. for 1 min), and then a fluorescence signal was collected.

5. The PCR was repeated directly by a common PCR instrument using a dilution factor and a log-phase cycle number of each sample determined by the qPCR above. The same parameters as the qPCR procedure above were adopted as much as possible, including temperature rise and fall rates. A log-phase cycle number of PCR for adding a barcode was determined.

6. A procedure for common PCR amplification was as follows: (1) 95° C. for 10 min; (2) 3cycles of (95° C. for 15 s, 62° C. for 30 s, and 72° C. for 1 min); (3) 2 cycles of (95° C. for 15 s, 64° C. for 30 s, and 72° C. for 1 min); (4) 11 *cycles of (95° C. for 15 s, 68° C. for 30 s, and 72° C. for 1 min) (A *cycle number was determined by the qPCR above); (5) 1 cycle of (95° C. for 15 s and 72° C. for 20 min); and (6) a first PCR tube was incubated at 72° C. for 18 min, and then taken out and immediately placed on ice to stop the Taq activity.

7. 30 μL of chloroform was added to the first PCR tube on ice (the chloroform was placed on ice for 30 min in advance), and then the first PCR tube was vortexed (for about 1 min).

8. The first PCR tube was centrifuged at 12,000 rpm and 4° C. for 15 min, and 25 μL of a resulting supernatant was taken and added to a second PCR tube (chloroform should not be touched, and a part of the supernatant was left).

9. The second PCR tube was carefully centrifuged in a mini centrifuge until the whole sample was precipitated to a bottom of the second PCR tube, and then placed in a PCR instrument at 50° C. for 10 min to allow the chloroform completely volatilized, otherwise the chloroform would inhibit a downstream enzyme reaction.

10. After PCR was completed, 2.5 μL of a diluted EXOI (Thermolabile) solution was added to the second PCR tube.

11. The second PCR tube was inverted up and down for thorough mixing, and then carefully centrifuged at 37° C. for 20 min and then at 42° C. for 10 min.

12. The ExoI was inactivated through a heat treatment at 60° C. for 15 min.

13. 3% agarose gel electrophoresis was conducted for 45 min to 60 min with a 50 bp marker, and whether a primer band disappeared was observed.

### IV. qPCR Amplification of a PCR Product with an Adapter Added (2× PCR Enzyme with UTP and without UDG)

1. qPCR of a PCR product with a barcode and an adapter added:

The PCR product obtained in the above experiment was diluted 50-fold to serve as a template. Primers used for the qPCR were designed as follows: a forward primer: an I5 sequencing adapter-containing sequence+an OUDI (I5 Index)+a sequence partially overlapping with a 5′ terminus of IUDI; and
a reverse primer: a Nextera I7 sequencing adapter-containing sequence+an OUDI (I7 Index sequence)+a sequence partially overlapping with a 5′ terminus of the IUDI in the above reverse primer.

The I5 Index and I7 Index sequences are selected from the I5 Index and I7 Index sequence sets in Table 2, but are different from the I5 Index and I7 Index sequences involved in the first PCR amplification.

An example of primers for the second PCR to add an OUDI are as follows:

a forward primer:
(SEQ ID NO: 2067)
AATGATACGGCGACCACCGAGATCTACACtacgaatcttACACTCTTTC
CCTACACGACGCTCTTCCGATCT;
and
a reverse primer:
(SEQ ID NO: 2068)
CTGTCTCTTATACACATCTCCGAGCCCACGAGACaccaagttacCTCGG
AGATGTGTATAAGAGACAG.

2. A qPCR procedure was as follows: (1) 95° C. for 10 min: (2) 3 cycles of (95° C. for 15 s, 62° C. for 30 s, and 72° C. for 1 min); (3) 2 cycles of (95° C. for 15 s, 64° C. for 30 s, and 72° C. for 1 min); and (4) 45 cycles of (95° C. for 15 s, 68° C. for 30 s, and 72° C. for 1 min), and then a fluorescence signal was collected. 3 replicates were set for each qPCR sample.

3. A dilution factor was calculated according to results of the above qPCR. Common PCR amplification (a small cycle number, which was intended to prevent the introduction of a human error), where 6 wells were set for each sample.

4. A procedure for the common PCR amplification was as follows: (1) 95° C. for 10 min; (2) 3 cycles of (95° C. for 15 s, 62° C. for 30 s, and 72° C. for 1 min); (3) 2 cycles of (95° C. for 15 s, 64° C. for 30 s, and 72° C. for 1 min); (4) 11 cycles of (95° C. for 15 s, 68° C. for 30 s, and 72° C. for 1 min); (5) 1 cycle of (95° C. for 15 s and 72° C. for 20 min); and (6) a PCR tube was incubated at 72° C. for 18 min, and then taken out and immediately placed on ice to stop the Taq activity. * A cycle number was determined by the qPCR described above (if it was impossible to continue, a resulting reaction system was stored at 4° C. or long-term stored at −20° C.).

5. After PCR was completed, 2.5 μL of diluted EXOI (Thermolabile) was added to the PCR tube.

6. The PCR tube was inverted up and down for thorough mixing, and then carefully centrifuged at 37° C. for 20 min and then at 42° C. for 10 min.

7. The ExoI was inactivated through a heat treatment at 60° C. for 15 min.

8. All samples were placed on ice, and 6 wells of PCR samples for each sample were mixed. Then qPCR was conducted (each sample was diluted 100,000-fold, and 3 replicates were set for a dilution; and 45 cycles were adopted).

### V. Precipitation and Gel Recovery of a Pooled Barcode and Adapter-Containing PCR Product

1. According to qPCR quantitative results, all samples were pooled in equal amounts and then vortexed for thorough mixing. Two replicates were set for the following experimental steps.

2. 700 μL of a pooled adapter-containing sample was added to a 1.5 mL EP tube.

3. 77 μL of a 3 M pH 5.2 sodium acetate solution was added to the EP tube.

4. 500 μL of isopropanol was added to the EP tube, and a resulting mixture was thoroughly mixed (the steps 2 to 4 needed to be conducted on ice).

5. The EP tube was placed at −20° C. or −80° C. for 1 h and then centrifuged in a centrifuge (with a cover handle facing outwards) at 15,000 g and 4° C. for 30 min.

6. When a first white DNA pellet was produced at a bottom of the EP tube, a resulting first supernatant was carefully poured off, and a first residual supernatant was carefully removed with a P200 pipette, during which the DNA pellet should not be touched to prevent DNA from being removed.

7. 500 μL of 70% room-temperature ethanol was added to the EP tube, and then the EP tube was placed at room temperature for 5 min.

8. The EP tube was centrifuged at 15,000 g and 4° C. for 30 min.

9. When a second white DNA pellet was produced at a bottom of the EP tube, a resulting second supernatant was carefully poured off, and a second residual supernatant was carefully removed with a P200 pipette.

10. The EP tube was horizontally placed in a clean bench (the EP tube was uncapped) and air-dried for about 10 min.

11. 60 μL of TE was added to the EP tube for dissolution.

12. A 1.5% agarose gel was prepared (the agarose gel had a thickness of about 1 cm, could hold 15 μL of a sample, and had a length twice a common length, namely about 15 cm).

13. Electrophoresis was conducted with a 3-4 pore gel for recovery. Notes: A dye band should run to a bottom of a gel, otherwise DNA fragments of different sizes cannot be fully separated. When a gel is cut, the smaller the gel band, the better, but a main band should be strictly included.

14. Recovered DNA was dissolved with 60 μL of TE. A DNA concentration of a resulting DNA solution was determined by electrophoresis, and then the DNA solution was stored at −20° C. for later use.

The precipitation and gel recovery were conducted with a mixed solution of all samples. If a PCR product had a high purity, the gel recovery was not required, and after the product was precipitated, the PCR primers were removed with the ExoI to obtain an isomiR library.

The constructed isomiR library was used to conduct NGS to obtain sequencing results.

### EXAMPLE 2

### Computer Processing of NGS Data

Raw NGS data were split into files of a number corresponding a number of samples (such as 200) in the pooled sample according to DUDI sequences. After the splitting, sequences irrelevant to mature miRNAs were removed by trimming software, and short RNA-seq data sets were directly processed by IsoMiRmap software to identify and quantify all isomiRs.

Batch effect analysis: Technical repeats can be used for batch effect analysis. A batch effect refers to the fact that a technical difference between different batches may result in significant heterogeneity between data of the different batches. If there is heterogeneity of replicated NGS data, it indicates poor repeatability, that is, there is a batch effect affecting the repeatability. A batch effect can be effectively removed by the batch effect removal software ComBat-seq. NGS data of seven batches were subjected to batch effect removal with the batch effect removal software ComBat-seq, and then calibrated into data in rpm (readings per million).

FIG. 2 is a scatter plot of PCA for NGS results of three replicated batches; NGS results of the three replicated batches each include 200 samples and 239 isomiRs. The scatter plot is obtained through dimensionality reduction by PCA. Data points of the three replicated batches are distinguished by different colors, as shown in FIG. 2.

Cluster overlap: The data points of the three replicated batches are blended with each other throughout the plot, indicating poor separation among the three batches. This blending may indicate that the intra-batch variability is similar to the inter-batch variability, which is a result of excellent repeatability of replicated experiments.

Inter-batch consistency: Since there is no obvious clustering of each batch, it may indicate that samples of all batches are consistent. If the batches should be the same under replicated experimental conditions, then it can be interpreted that the experiment is repeatable.

No batch effect: Since there is no obvious independent clustering to separate the batches, it means that there is no significant batch effect. A batch effect is typically manifested as independent clustering for each batch.

Potential outliers: It seems that there is no significant outlier far from a main concentration point in the plot, which further supports the concept of repeatability.

It should be noted that, while PCA can provide a visual representation for data variability and clustering, PCA cannot replace the statistical testing for quantitatively assessing the repeatability. For comprehensive analysis, an additional statistical method should be adopted.

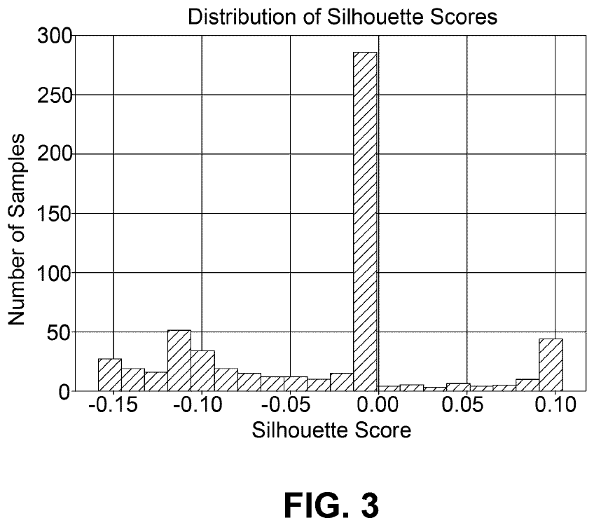

FIG. 3 is a histogram of Silhouette scores of PCA for NGS results of three replicated batches. Silhouette analysis was conducted with the PCA results in this figure, and 600 Silhouette scores were obtained, with one Silhouette score for each sample. This histogram shows a distribution of these scores across different ranges or groups.

Batch effect analysis: Technical repeats can be used for batch effect analysis. A batch effect refers to the fact that a technical difference between different batches may result in significant heterogeneity between data of the different batches. If there is heterogeneity of replicated NGS data, it indicates poor repeatability, that is, there is a batch effect affecting the repeatability. A batch effect can be effectively removed by the batch effect removal software ComBat-seq. Seven batches of data were subjected to Procrustes analysis before and after batch effect removal with ComBat-seq: An output of the Procrustes analysis provided several key pieces of information for comparing PCA results of two groups before and after batch effect removal.

Biological repeatability: The NGS has high repeatability, indicating that the expression of isomiRs can be accurately quantified. Only the high technical repeatability can guarantee the biological repeatability.

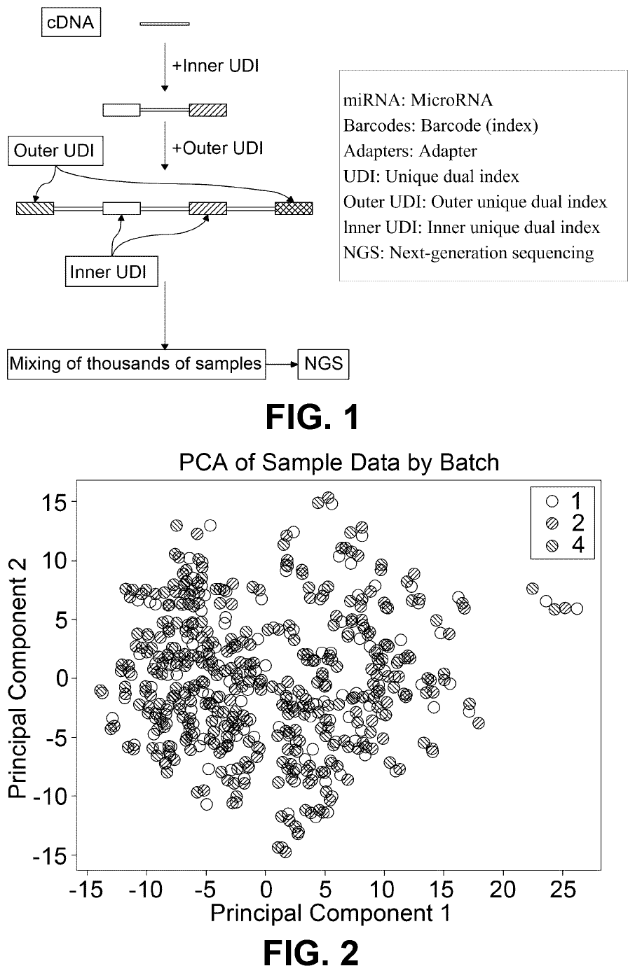

The NGS method was used to detect plasma isomiRs in 300 gastric cancer samples and 300non-gastric cancer samples (including health and gastric disease samples). Each batch of sequencing involved 100 gastric cancer samples and 100 non-gastric cancer samples (healthy or gastric disease samples). Three NGS replicates were set for each sample (starting from RNA extraction, RT, or cDNA). In order to verify the biological repeatability, the sequencing of a same sample was repeated three or more times. Machine learning models were built with different batches of sequencing results, respectively, and then used to predict for each other. FIG. 1 shows the confusion matrix results of machine learning. A confusion matrix, also known as a contingency table or an error matrix, is a specific matrix to present the performance of a supervised machine learning algorithm. The name “confusion matrix” comes from the fact that a confusion matrix can very easily indicate whether there is confusion between two categories and a confusion degree between two categories.

### EXAMPLE 3

### Construction of a Machine Learning Model

A t-Test P value of an expression difference of each isomiR between 100 gastric cancer samples and 100 non-gastric cancer samples was calculated, and isomiRs were ranked from small to large according to t-Test P values. 239 isomiRs were selected and correlations among these different isomiRs were further calculated; and then isomiRs highly correlated with other isomiRs were removed. The remaining data were subjected to machine-learning classification with different classifiers to find the optimal classifier. A variety of classifiers were adopted. The data were split by each classifier into two parts: 80% for model training and 20% for model validation. An SVM algorithm was determined to be the optimal.

A machine learning model for auxiliary diagnosis of gastric cancer was established by the SVM algorithm: The data were divided into two parts: 80% for model training (a training set) and 20% for model validation (a test set). Samples were divided into a training set and a test set. Replicate samples only exist in the training set or the test set, that is, different replicates of a same sample cannot exist in both the training set and the test set, otherwise there will be information leakage and an evaluation of a model will be too high.

Optimization of an SVM algorithm model: Parameters of the SVM algorithm were debugged to find the optimal parameters. The parameters of the SVM algorithm were optimized through grid search. Numerical ranges of the parameters were as follows: gamma=2⁽⁻⁸⁻¹⁾ and cost=2⁽⁰⁻⁴⁾. In this way, the gamma had 10 values and the cost had 5 values, that is, there were 50 combinations. Each combination was subjected to 10-fold cross-validation, that is, the training set was divided into 10 parts, where 9 parts were used in turn as training data and 1 part was used as test data for trials. Each trial led to a corresponding error rate. An average error rate for each combination was obtained through 10 trials. A gamma/cost combination with a minimum average error rate was determined as the optimal parameters of the SVM algorithm. Since a final diagnosis model was obtained through 500 (50×10) trials, overfitting could be avoided. The overfitting is a phenomenon in which a trained model performs well on a training set but poor on a test set.

Model evaluation: There are many different indexes to evaluate a machine learning algorithm. Default evaluation criteria for classification problems are accuracy and Kappa. The Kappa is similar to the accuracy, but is calibrated by a random baseline of a data set. A kappa value represents both consistency and classification accuracy. The closer the kappa value to 1, the more excellent the consistency. Usually, a kappa value of 0.75 or more means that a consistency result is satisfactory, and a kappa value of 0.8 to 1 means that results are almost completely consistent. Accuracy, Kappa, and other evaluation indexes could be described by a confusion matrix and an ROC curve.

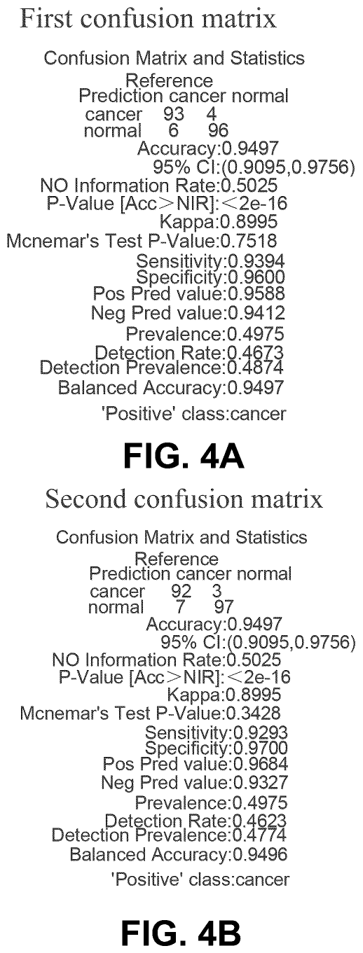

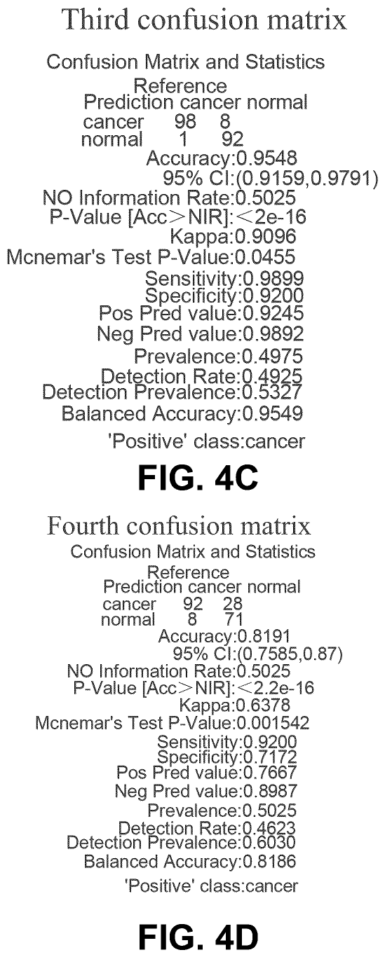

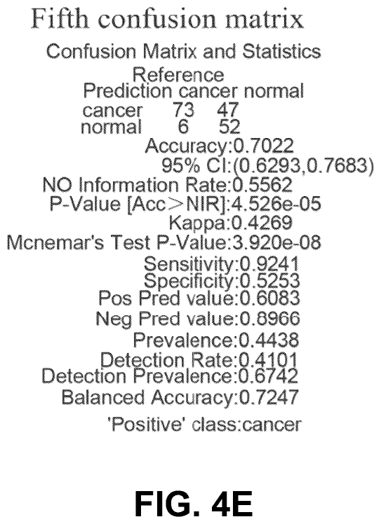

FIGS. 4A-4E show the comparison of confusion matrices for machine learning. In order to verify the biological repeatability, the sequencing of a same sample was repeated three or more times in the present disclosure. NGS results of a first batch were used to build a first model, and then the first model was used to predict NGS data of a second batch (a first confusion matrix). Conversely, the NGS data of the second batch were used to build a second model, and then the second model was used to predict the NGS data of the first batch (a second confusion matrix). To demonstrate the high repeatability of multiple times of replicated NGS, the second model established was used to predict NGS data of a third batch (a third confusion matrix). What is more important is whether the machine learning model constructed is universal, that is, whether the machine learning model can be used to predict NGS data of different samples. NGS data of another batch of completely different samples were successfully predicted by the second model above (a fourth confusion matrix). NGS data of a second batch of completely different samples were also successfully predicted by the same model (a fifth confusion matrix).

Two confusion matrices for mutual authentication both had an accuracy of 95% and a sensitivity and specificity of 90% or more, indicating that NGS data of the two times were highly similar, that is, the biological repeatability was high. NGS data of the third batch (the third confusion matrix) predicted by the second model also had an accuracy of 95% and a sensitivity and specificity of 90% or more, indicating that multiple times of NGS of a same sample had high biological repeatability. What is more important is whether the machine learning model constructed is universal, that is, whether the machine learning model can be used to predict NGS data of different samples. NGS data of another batch of completely different samples were successfully predicted by the second model above (a fourth confusion matrix). NGS data of a second batch of completely different samples were also successfully predicted by the same model (a fifth confusion matrix). While a confusion matrix has lower accuracy, sensitivity, and specificity than the prediction of NGS data of the same samples from different batches, it is expected, and given that a large amount of data is required for modeling by machine learning (because gastric cancer has high genetic heterogeneity), 200 samples are insufficient. However, importantly, P values of confusion matrices are low, indicating that prediction results are very statistically significant and cannot be coincidental. These experimental results fully show that the artificial intelligent diagnosis technology for a tumor based on NGS in the present disclosure can effectively distinguish between gastric cancer and non-gastric cancer diseases (gastritis, gastric ulcer, gastric erosion, and other gastric discomforts), and has excellent biological repeatability (when different samples are adopted). The sensitivity and specificity of prediction by the technology both can reach 90% or more. It indicates that the double unique dual indexing technology for multiplex NGS of the present disclosure has both high technical repeatability and high biological repeatability, and can detect a natural variation of a biological sample, that is, specific detection results. If a detection is not specific, a non-specific signal masks a specific signal, and thus it is impossible to obtain such a specific detection result.

The above-mentioned NGS results prove from the technical repeatability and the biological repeatability that the NGS library construction technology for isomiRs developed in the present disclosure has high repeatability and can be used for artificial intelligent diagnosis of a tumor.

The above are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

1. A primer set for amplification of a microRNA isoform (isomiR), comprising a universal sequence and a 5′-terminus amplification primer linked sequentially to a partial sequence of a 5′ terminus of a microRNA (miRNA).

2. The primer set for amplification of an isomiR according to claim 1, wherein the miRNA comprises at least one selected from the group consisting of the following: hsa-miR-21-5p, hsa-miR-223-3p, hsa-miR-223-5p, hsa-miR-186-5p, hsa-miR-18a-5p, hsa-miR-146b-5p, hsa-miR-624-5p, hsa-miR-106b-5p, hsa-miR-340-5p, hsa-miR-20a-5p, hsa-miR-451a, hsa-miR-7976, hsa-miR-2355-3p, hsa-miR-301a-3p, hsa-miR-144-5p, hsa-miR-151a-3p, hsa-miR-3200-5p, hsa-miR-1537-3p, hsa-miR-500a-5p, hsa-miR-127-3p, hsa-miR-570-3p, hsa-miR-130b-5p, hsa-miR-503-5p, hsa-miR-551a, hsa-miR-409-3p, hsa-miR-330-3p, hsa-miR-889-3p, hsa-miR-625-5p, hsa-miR-542-3p, hsa-miR-582-3p, hsa-miR-381-3p, hsa-miR-495-3p, hsa-miR-103a-1-5p, hsa-miR-450b-5p, hsa-miR-429, hsa-miR-576-5p, hsa-miR-148b-3p, hsa-miR-320c, hsa-miR-4286, hsa-miR-126-3p, hsa-miR-152-3p, hsa-miR-144-3p, hsa-miR-195-5p, hsa-let-7a-5p, hsa-miR-378f, hsa-miR-126-5p, hsa-miR-26a-5p, hsa-miR-29a-3p, hsa-miR-181a-5p, hsa-miR-32-5p, hsa-miR-142-3p, hsa-miR-29c-3p, hsa-miR-424-5p, hsa-miR-192-5p, hsa-miR-143-3p, hsa-miR-30c-5p, hsa-miR-146a-5p, hsa-miR-101-3p, hsa-miR-19b-3p, hsa-miR-33b-5p, hsa-miR-378a-3p, hsa-miR-22-3p, hsa-miR-107, hsa-miR-497-5p, hsa-miR-15a-3p, hsa-miR-188-5p, hsa-let-7d-3p, hsa-miR-132-3p, hsa-miR-151a-5p, hsa-miR-194-5p, hsa-miR-99a-5p, hsa-miR-125b-5p, hsa-miR-25-3p, hsa-miR-103a-3p, hsa-miR-1285-3p, hsa-miR-7977, hsa-miR-30b-5p, hsa-miR-363-3p, hsa-miR-93-5p, hsa-miR-375-3p, hsa-miR-99b-5p, hsa-miR-193b-3p, hsa-miR-324-3p, hsa-miR-193a-3p, hsa-miR-342-3p, hsa-miR-484, hsa-miR-532-3p, hsa-miR-210-3p, hsa-miR-2110, hsa-miR-296-5p, hsa-miR-1307-5p, hsa-miR-19a-3p, hsa-miR-139-5p, hsa-miR-3665, hsa-miR-RG-84, hsa-miR-4454, and hsa-let-7b-5p; and
nucleotide sequences of corresponding 5′-terminus amplification primers are shown in SEQ ID NO: 1 to SEQ ID NO: 97, respectively.

3. The primer set for amplification of an isomiR according to claim 1, further comprising a second polymerase chain reaction (PCR) preamplification primer pair and/or a third PCR preamplification primer pair;
the second PCR preamplification primer pair comprises a transition primer and a reverse primer for amplifying the isomiR;
a nucleotide sequence of the transition primer is shown in SEQ ID NO: 99;
a nucleotide sequence of the reverse primer for amplifying the isomiR is shown in SEQ ID NO: 100;
the third PCR preamplification primer pair comprises a 5′ universal primer and a 3′ universal primer;
a nucleotide sequence of the 5′ universal primer is shown in SEQ ID NO: 101; and
a nucleotide sequence of the 3′ universal primer is shown in SEQ ID NO: 102.

4. A method for amplifying an isomiR, comprising the following steps:
extracting total RNA from each of a gastric cancer sample and a non-gastric cancer sample, and reverse-transcribing the total RNA into cDNA;
with the cDNA as a template, conducting a first PCR preamplification using the primer set to obtain a first preamplification product, the primer set comprising a universal sequence and a 5′-terminus amplification primer linked sequentially to a partial sequence of a 5′ terminus of a microRNA (miRNA), wherein the miRNA comprises at least one selected from the group consisting of the following: hsa-miR-21-5p, hsa-miR-223-3p, hsa-miR-223-5p, hsa-miR-186-5p, hsa-miR-18a-5p, hsa-miR-146b-5p, hsa-miR-624-5p, hsa-miR-106b-5p, hsa-miR-340-5p, hsa-miR-20a-5p, hsa-miR-451a, hsa-miR-7976, hsa-miR-2355-3p, hsa-miR-301a-3p, hsa-miR-144-5p, hsa-miR-151a-3p, hsa-miR-3200-5p, hsa-miR-1537-3p, hsa-miR-500a-5p, hsa-miR-127-3p, hsa-miR-570-3p, hsa-miR-130b-5p, hsa-miR-503-5p, hsa-miR-551a, hsa-miR-409-3p, hsa-miR-330-3p, hsa-miR-889-3p, hsa-miR-625-5p, hsa-miR-542-3p, hsa-miR-582-3p, hsa-miR-381-3p, hsa-miR-495-3p, hsa-miR-103a-1-5p, hsa-miR-450b-5p, hsa-miR-429, hsa-miR-576-5p, hsa-miR-148b-3p, hsa-miR-320c, hsa-miR-4286, hsa-miR-126-3p, hsa-miR-152-3p, hsa-miR-144-3p, hsa-miR-195-5p, hsa-let-7a-5p, hsa-miR-378f, hsa-miR-126-5p, hsa-miR-26a-5p, hsa-miR-29a-3p, hsa-miR-181a-5p, hsa-miR-32-5p, hsa-miR-142-3p, hsa-miR-29c-3p, hsa-miR-424-5p, hsa-miR-192-5p, hsa-miR-143-3p, hsa-miR-30c-5p, hsa-miR-146a-5p, hsa-miR-101-3p, hsa-miR-19b-3p, hsa-miR-33b-5p, hsa-miR-378a-3p, hsa-miR-22-3p, hsa-miR-107, hsa-miR-497-5p, hsa-miR-15a-3p, hsa-miR-188-5p, hsa-let-7d-3p, hsa-miR-132-3p, hsa-miR-151a-5p, hsa-miR-194-5p, hsa-miR-99a-5p, hsa-miR-125b-5p, hsa-miR-25-3p, hsa-miR-103a-3p, hsa-miR-1285-3p, hsa-miR-7977, hsa-miR-30b-5p, hsa-miR-363-3p, hsa-miR-93-5p, hsa-miR-375-3p, hsa-miR-99b-5p, hsa-miR-193b-3p, hsa-miR-324-3p, hsa-miR-193a-3p, hsa-miR-342-3p, hsa-miR-484, hsa-miR-532-3p, hsa-miR-210-3p, hsa-miR-2110, hsa-miR-296-5p, hsa-miR-1307-5p, hsa-miR-19a-3p, hsa-miR-139-5p, hsa-miR-3665, hsa-miR-RG-84, hsa-miR-4454, and hsa-let-7b-5p; and
nucleotide sequences of corresponding 5′-terminus amplification primers are shown in SEQ ID NO: 1 to SEQ ID NO: 97, respectively;
with the first preamplification product as a template, conducting a second PCR preamplification using the transition primer and the reverse primer for amplifying the isomiR in the primer set according to claim 3 to obtain a second preamplification product; and
with the second preamplification product as a template, conducting a third PCR preamplification using the 5′ universal primer and the 3′ universal primer in the primer set according to claim 3 to obtain a third preamplification product, which is the isomiR.

5. A double unique dual indexing amplification primer set for construction of a high-throughput sample library for next-generation sequencing (NGS), comprising primers for adding an inner double unique dual index (DUDI) and primers for adding an outer DUDI and a sequencing adapter,
wherein a forward primer among the primers for adding an inner DUDI is obtained by linking a DNA fragment shown in SEQ ID NO: 2055, an I5 Index sequence, and a DNA fragment shown in SEQ ID NO: 2056 sequentially;
a reverse primer among the primers for adding an inner DUDI is obtained by linking a DNA fragment shown in SEQ ID NO: 2057, an I7 Index sequence, and a DNA fragment shown in SEQ ID NO: 2058 sequentially;
a forward primer among the primers for adding an outer DUDI and a sequencing adapter is obtained by linking a DNA fragment shown in SEQ ID NO: 2059, the I5 Index sequence, and a DNA fragment shown in SEQ ID NO: 2060 sequentially;
a reverse primer among the primers for adding an outer DUDI and a sequencing adapter is obtained by linking a DNA fragment shown in SEQ ID NO: 2061, the I7 Index sequence, and a DNA fragment shown in SEQ ID NO: 2062 sequentially;
a nucleotide sequence of the I5 Index sequence is one selected from the group consisting of SEQ ID NO: 103 to SEQ ID NO: 1078; and
a nucleotide sequence of the I7 Index sequence is one selected from the group consisting of SEQ ID NO: 1079 to SEQ ID NO: 2054.

6. A kit for construction of a high-throughput sample library for NGS, comprising the primer set for amplification of an isomiR according to claim 1, the double unique dual indexing amplification primer set, the double unique dual indexing amplification primer set comprises primers for adding an inner double unique dual index (DUDI) and primers for adding an outer DUDI and a sequencing adapter,
wherein a forward primer among the primers for adding an inner DUDI is obtained by linking a DNA fragment shown in SEQ ID NO: 2055, an I5 Index sequence, and a DNA fragment shown in SEQ ID NO: 2056 sequentially;
a reverse primer among the primers for adding an inner DUDI is obtained by linking a DNA fragment shown in SEQ ID NO: 2057, an I7 Index sequence, and a DNA fragment shown in SEQ ID NO: 2058 sequentially:
a forward primer among the primers for adding an outer DUDI and a sequencing adapter is obtained by linking a DNA fragment shown in SEQ ID NO: 2059, the I5 Index sequence, and a DNA fragment shown in SEQ ID NO: 2060 sequentially;
a reverse primer among the primers for adding an outer DUDI and a sequencing adapter is obtained by linking a DNA fragment shown in SEQ ID NO: 2061, the I7 Index sequence, and a DNA fragment shown in SEQ ID NO: 2062 sequentially;
a nucleotide sequence of the I5 Index sequence is one selected from the group consisting of SEQ ID NO: 103 to SEQ ID NO: 1078; and
a nucleotide sequence of the I7 Index sequence is one selected from the group consisting of SEQ ID NO: 1079 to SEQ ID NO: 2054; and 2× boost mix,
wherein the 2× Boost mix comprises the following components: water as a solvent, Tris-HCl: 70 mmol/L to 80 mmol/L, (NH₄)₂SO₄: 15 mmol/L to 25 mmol/L, Triton-100: 0.08% to 0.12% in a volume concentration, MgCl₂: 2 mmol/L to 3 mmol/L, dNTPs: 150 μmol/L to 250 μmol/L, trehalose: 190 mmol/L to 210 mmol/L, and hot-start Taq DNA polymerase: 45,000 U/L to 55,000 U/L; a pH of the Tris-HCl is 8.5 to 9.0; and
the dNTPs refers to a dNTP mixed solution that comprises UDG and does not comprise dUTP.

7. A method for construction of a high-throughput sample library for NGS, comprising the following steps:
with the third preamplification product obtained by the method according to claim 4 as a template, conducting a first PCR amplification using the primers for adding an inner DUDI in the double unique dual indexing amplification primer set to obtain an inner unique dual index (IUDI)-containing PCR product; the double unique dual indexing amplification primer set comprises primers for adding an inner double unique dual index (DUDI) and primers for adding an outer DUDI and a sequencing adapter,
wherein a forward primer among the primers for adding an inner DUDI is obtained by linking a DNA fragment shown in SEQ ID NO: 2055, an I5 Index sequence, and a DNA fragment shown in SEQ ID NO: 2056 sequentially;
a reverse primer among the primers for adding an inner DUDI is obtained by linking a DNA fragment shown in SEQ ID NO: 2057, an I7 Index sequence, and a DNA fragment shown in SEQ ID NO: 2058 sequentially;
a forward primer among the primers for adding an outer DUDI and a sequencing adapter is obtained by linking a DNA fragment shown in SEQ ID NO: 2059, the I5 Index sequence, and a DNA fragment shown in SEQ ID NO: 2060 sequentially;
a reverse primer among the primers for adding an outer DUDI and a sequencing adapter is obtained by linking a DNA fragment shown in SEQ ID NO: 2061, the I7 Index sequence, and a DNA fragment shown in SEQ ID NO: 2062 sequentially;
a nucleotide sequence of the I5 Index sequence is one selected from the group consisting of SEQ ID NO: 103 to SEQ ID NO: 1078; and a nucleotide sequence of the I7 Index sequence is one selected from the group consisting of SEQ ID NO: 1079 to SEQ ID NO: 2054;
with the IUDI-containing PCR product as a template, conducting a second PCR amplification using the primers for adding an outer DUDI and a sequencing adapter in the double unique dual indexing amplification primer set to obtain a DUDI-containing PCR product; and pooling to obtain a sequencing library; the double unique dual indexing amplification primer set comprises primers for adding an inner double unique dual index (DUDI) and primers for adding an outer DUDI and a sequencing adapter,
wherein a forward primer among the primers for adding an inner DUDI is obtained by linking a DNA fragment shown in SEQ ID NO: 2055, an I5 Index sequence, and a DNA fragment shown in SEQ ID NO: 2056 sequentially;
a reverse primer among the primers for adding an inner DUDI is obtained by linking a DNA fragment shown in SEQ ID NO: 2057, an I7 Index sequence, and a DNA fragment shown in SEQ ID NO: 2058 sequentially;
a forward primer among the primers for adding an outer DUDI and a sequencing adapter is obtained by linking a DNA fragment shown in SEQ ID NO: 2059, the I5 Index sequence, and a DNA fragment shown in SEQ ID NO: 2060 sequentially;
a reverse primer among the primers for adding an outer DUDI and a sequencing adapter is obtained by linking a DNA fragment shown in SEQ ID NO: 2061, the I7 Index sequence, and a DNA fragment shown in SEQ ID NO: 2062 sequentially;
a nucleotide sequence of the I5 Index sequence is one selected from the group consisting of SEQ ID NO: 103 to SEQ ID NO: 1078; and
a nucleotide sequence of the I7 Index sequence is one selected from the group consisting of SEQ ID NO: 1079 to SEQ ID NO: 2054.

8. The method for construction of a high-throughput sample library for NGS according to claim 6, further comprising: precipitating a pooled DUDI-containing PCR product, and removing PCR primers from a product precipitate with an ExoI enzyme to obtain the sequencing library.

9. A method of use of the primer set for amplification of an isomiR according to claim 1 in construction of a prediction model for artificial intelligent diagnosis of a tumor based on NGS.

10. The method according to claim 9, wherein the tumor comprises gastric cancer.

11. A kit for construction of a high-throughput sample library for NGS, comprising the primer set for amplification of an isomiR according to claim 2, the double unique dual indexing amplification primer set, the double unique dual indexing amplification primer set comprises primers for adding an inner double unique dual index (DUDI) and primers for adding an outer DUDI and a sequencing adapter,
wherein a forward primer among the primers for adding an inner DUDI is obtained by linking a DNA fragment shown in SEQ ID NO: 2055, an I5 Index sequence, and a DNA fragment shown in SEQ ID NO: 2056 sequentially;
a reverse primer among the primers for adding an inner DUDI is obtained by linking a DNA fragment shown in SEQ ID NO: 2057, an I7 Index sequence, and a DNA fragment shown in SEQ ID NO: 2058 sequentially;
a forward primer among the primers for adding an outer DUDI and a sequencing adapter is obtained by linking a DNA fragment shown in SEQ ID NO: 2059, the I5 Index sequence, and a DNA fragment shown in SEQ ID NO: 2060 sequentially;
a reverse primer among the primers for adding an outer DUDI and a sequencing adapter is obtained by linking a DNA fragment shown in SEQ ID NO: 2061, the I7 Index sequence, and a DNA fragment shown in SEQ ID NO: 2062 sequentially;
a nucleotide sequence of the I5 Index sequence is one selected from the group consisting of SEQ ID NO: 103 to SEQ ID NO: 1078; and
a nucleotide sequence of the I7 Index sequence is one selected from the group consisting of SEQ ID NO: 1079 to SEQ ID NO: 2054; and 2× boost mix,
wherein the 2× Boost mix comprises the following components: water as a solvent, Tris-HCl: 70 mmol/L to 80 mmol/L, (NH₄)₂SO₄: 15 mmol/L to 25 mmol/L, Triton-100: 0.08% to 0.12% in a volume concentration, MgCl₂: 2 mmol/L to 3 mmol/L, dNTPs: 150 μmol/L to 250 μmol/L, trehalose: 190 mmol/L to 210 mmol/L, and hot-start Tag DNA polymerase: 45,000 U/L to 55,000 U/L; a pH of the Tris-HCl is 8.5 to 9.0; and
the dNTPs refers to a dNTP mixed solution that comprises UDG and does not comprise dUTP.

12. A kit for construction of a high-throughput sample library for NGS, comprising the primer set for amplification of an isomiR according to claim 3, the double unique dual indexing amplification primer set, the double unique dual indexing amplification primer set comprises primers for adding an inner double unique dual index (DUDI) and primers for adding an outer DUDI and a sequencing adapter,
wherein a forward primer among the primers for adding an inner DUDI is obtained by linking a DNA fragment shown in SEQ ID NO: 2055, an I5 Index sequence, and a DNA fragment shown in SEQ ID NO: 2056 sequentially;
a reverse primer among the primers for adding an inner DUDI is obtained by linking a DNA fragment shown in SEQ ID NO: 2057, an I7 Index sequence, and DNA fragment shown in SEQ ID NO: 2058 sequentially;
a forward primer among the primers for adding an outer DUDI and a sequencing adapter is obtained by linking a DNA fragment shown in SEQ ID NO: 2059, the I5 Index sequence, and a DNA fragment shown in SEQ ID NO: 2060 sequentially;
a reverse primer among the primers for adding an outer DUDI and a sequencing adapter is obtained by linking a DNA fragment shown in SEQ ID NO: 2061, the I7 Index sequence, and a DNA fragment shown in SEQ ID NO: 2062 sequentially;
a nucleotide sequence of the I5 Index sequence is one selected from the group consisting of SEQ ID NO: 103 to SEQ ID NO: 1078; and
a nucleotide sequence of the I7 Index sequence is one selected from the group consisting of SEQ ID NO: 1079 to SEQ ID NO: 2054; and 2× boost mix,
wherein the 2× Boost mix comprises the following components: water as a solvent, Tris-HCl: 70 mmol/L to 80 mmol/L, (NH₄)₂SO₄: 15 mmol/L to 25 mmol/L, Triton-100: 0.08% to 0.12% in a volume concentration, MgCl₂: 2 mmol/L to 3 mmol/L, dNTPs: 150 μmol/L to 250 μmol/L, trehalose: 190 mmol/L to 210 mmol/L, and hot-start Tag DNA polymerase: 45,000 U/L to 55,000 U/L; a pH of the Tris-HCl is 8.5 to 9.0; and
the dNTPs refers to a dNTP mixed solution that comprises UDG and does not comprise dUTP.

13. A method of use of the primer set for amplification of an isomiR according to claim 2 in construction of a prediction model for artificial intelligent diagnosis of a tumor based on NGS.

14. A method of use of the primer set for amplification of an isomiR according to claim 3 in construction of a prediction model for artificial intelligent diagnosis of a tumor based on NGS.

15. A method of use of an isomiR amplified by the method according to claim 4 in construction of a prediction model for artificial intelligent diagnosis of a tumor based on NGS.

16. A method of use of the double unique dual indexing amplification primer set according to claim 5 in construction of a prediction model for artificial intelligent diagnosis of a tumor based on NGS.

17. The method according to claim 13, wherein the tumor comprises gastric cancer.

18. The method according to claim 14, wherein the tumor comprises gastric cancer.

19. The method according to claim 15, wherein the tumor comprises gastric cancer.

20. The method according to claim 16, wherein the tumor comprises gastric cancer.
